(19) United States
(12) Patent Application Publication
(10) Pub. No.: US 2020/0323834 A1
(43) Pub. Date: Oct. 15, 2020

(54) HYDROPHOBIC ACID ADDITION SALTS AND PHARMACEUTICAL FORMULATIONS THEREOF

(71) Applicant: Theracaine LLC, San Francisco, CA (US)

(72) Inventor: Samuel P. Sawan, San Rafael, CA (US)

(21) Appl. No.: 16/860,391

(22) Filed: Apr. 28, 2020

Related U.S. Application Data
(63) Continuation of application No. PCT/US18/58120, filed on Oct. 30, 2018.
(60) Provisional application No. 62/578,857, filed on Oct. 30, 2017.
(60) Provisional application No. 62/578,803, filed on Oct. 30, 2017.
(60) Provisional application No. 62/589,014, filed on Nov. 21, 2017.
(60) Provisional application No. 62/589,021, filed on Nov. 21, 2017.
(60) Provisional application No. 62/589,108, filed on Nov. 21, 2017.
(60) Provisional application No. 62/589,134, filed on Nov. 21, 2017.

(51) Int. Cl. A61K 31/445; A61K 31/167; A61K 47/14; A61K 9/70; A61K 47/34

(52) U.S. Cl. CPC A61K 31/445; A61K 31/167; A61K 47/34; A61K 9/7007; A61K 47/14

(57) ABSTRACT

The invention provides hydrohphobic drug salts and pharmaceutical compositions comprising such salts. The invention fourther provides compositions for delivering poorly soluble drugs, including hydrophobic drug salts.

### RELATED APPLICATIONS

This application is a continuation of International Application No. PCT/US18/58120, which designated the United States and was filed on Oct. 30, 2018, published in English, which claims the benefit of U.S. Provisional Application No. 62/578,857, filed on Oct. 30, 2017, U.S. Provisional Application No. 62/578,803, filed on Oct. 30, 2017, U.S.

Provisional Application No. 62/589,014, filed on Nov. 21, 2017, U.S. Provisional Application No. 62/589,021, filed on Nov. 21, 2017, U.S. Provisional Application No. 62/589,108, filed on Nov. 21, 2017 and U.S. Provisional Application No. 62/589,134, filed on Nov. 21, 2017. The entire teachings of the above applications are incorporated herein by reference.

### BACKGROUND OF THE INVENTION

The physicochemical characteristics and economical state of a medicinal drug can be manipulated and improved by conversion to a salt form. Selecting the appropriate salt is considered to be a very important step since each salt shows distinctive properties to the parent drug. Usually the salt-forming agents are selected by testing and experience according to the cost of raw materials, simplicity of crystallization and the amount of yield produced.

It has been estimated that approximately 50% of all drug molecules marketed as medicinal products are administered in a form of salts. This simple statistic shows that salt formation of drug substances is a central pre-formulation process and it must be associated with significant advantages. Certainly, many drug molecules are characterized by undesirable physicochemical properties that can be effectively improved by converting a basic or acidic drug into a salt form.

Salt formation offers many advantages to the pharmaceutical products as it can improve the solubility, dissolution rate, permeability and efficacy of the drug. In addition, salts can help in the improvement of the hydrolytic and thermal stability. Also, salts play an important role in targeted drug delivery of dosage form (e.g. in the cases of controlled release dosage forms).

In one embodiment salt formation involves, in essence pairing the parent drug molecule with an appropriate counterion. The essential prerequisite is the presence of a basic functional group in the drug's structure that allow sufficient ionic interaction between the drug and the acid. The charged groups in the structure of the drug and the conjugate base of the acid are attracted by ionic intermolecular forces. At favorable thermodynamic conditions, the salt is precipitated in the crystallized form.

The choice of the salt forming agent is dictated by a number of criteria that the salt is intended to meet. Formulation (dosage form) type may influence this choice—for solid dosage forms, oral solutions, and injectables, highly soluble hydrochlorides and mesylates, besylates and other forms can be chosen. Alternatively, for suspensions or otherwise slow drug release profiles, relatively hydrophobic counterions may be preferred such as those described herein.

### SUMMARY OF THE INVENTION

In a first embodiment, the invention provides acid addition salts of a basic therapeutic agent wherein the acid is represented by Formula 1 or Formula 2:

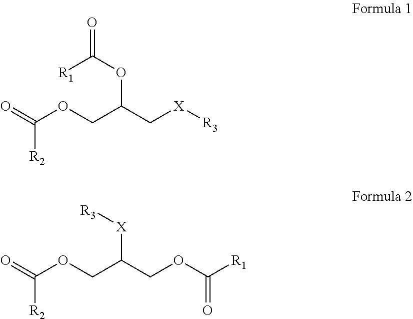

wherein R₁ and R₂ are the same or different and are each independently optionally substituted C₁-C₂₄-alkyl, optionally substituted C₂-C₂₄-alkenyl, optionally substituted C₂-C₂₄-alkynyl, optionally substituted C₃-C₁₂-cycloalkyl or optionally substituted aryl; X is O or absent and R₃ is SO₃H, P(O)(OR₄)OH or C(O)OH; or X is O and R₃ is —CH₂C(O)OH, —CH₂C(O)SH or —CH₂C(S)SH; or X is absent and R₃ is C(O)SH or C(S)SH. R₄ is hydrogen; optionally substituted alkyl, preferably optionally substituted C₁-C₆-alkyl; optionally substituted C₃-C₁₂-cycloalkyl; optionally substituted alkenyl, preferably optionally substituted C₂-C₆-alkenyl; optionally substituted alkynyl, preferably optionally substituted C₂-C₆-alkynyl; or optionally substituted aryl. R₄ is preferably hydrogen or C₁-C₆-alkyl and more preferably hydrogen.

The invention also provides pharmaceutical compositions comprising a pharmaceutically acceptable carrier or excipient and an acid addition salt of the invention.

The invention further includes methods of treating a disease or disorder in a subject in need thereof, comprising administering to the subject an effective amount of an acid addition salt of the invention.

In a second embodiment, the present invention provides hydrophobic pharmaceutical salts. In one embodiment, the hydrophobic pharmaceutical salt is an acid addition salt of a basic drug wherein the acid is an organic acid which comprises at least six carbon atoms. The organic acid can be, for example, a carboxylic acid, a sulfonic acid, a phosphonic acid, a sulfuric acid ester or a phosphoric acid ester, each having six or more carbon atoms. Preferably, the acid comprises an optionally substituted alkyl, alkenyl, alkynyl or aryl group having at least six carbon atoms, for example from six to about 30 carbon atoms. In certain embodiments, the acid is an optionally substituted C₆-C₃-alkylcarboxylic acid, an optionally substituted C₆-C₃₀-alkylsulfonic acid, an optionally substituted C₆-C₃₀-alkylphosphonic acid, an optionally substituted sulfuric acid C₆-C₃₀-alkyl ester or an optionally substituted phosphoric acid C₆-C₃₀-alkyl ester. In certain embodiments, the acid is an optionally substituted C₆-C₃₀-alkenylcarboxylic acid, an optionally substituted C₆-C₃₀-alkenylsulfonic acid, an optionally substituted C₆-C₃₀-alkenylphosphonic acid, an optionally substituted sulfuric acid C₆-C₃₀-alkenyl ester or an optionally substituted phosphoric acid C₆-C₃-alkenyl ester. In yet other embodiments, the acid is an optionally substituted C₆-C₃₀-alkynylcarboxylic acid, an optionally substituted C₆-C₃₀-alkynylsulfonic acid, an optionally substituted C₆-C₃-alkynylphosphonic acid, an optionally substituted sulfuric acid C₆-C₃₀-alkynyl ester or an optionally substituted phosphoric acid C₆-C₃-alkynyl ester. In further embodiments, the acid is an optionally substituted C₆-C₁₄-arylcarboxylic acid, an optionally substituted C₆-C₁₄-arylsulfonic acid, an optionally substituted C₆-C₁₄-arylphosphonic acid, an optionally substituted sulfuric acid C₆-C₁₄-aryl ester or an optionally substituted phosphoric acid C₆-C₁₄-aryl ester.

In certain embodiments, the drug has a basic functional group, for example, a primary, secondary or tertiary amine, which is protonated in the salt. In other embodiments, the drug has a quaternary ammonium group.

The drug can be an addiction treatment, such as naltrexone, varenicline or buprenorphine; an antibiotic, such as neomycin, vancomycin, linezolid or clindamycin; an anti-emetic, such as granisetron or ondansetron; a psychoactive agent, such as aripiprazole, respiridone, olanzapine, clozapine, haloperidol or chlorpromazine; a bronchodilator, such as iprotropium or tiotropium; or a urinary drug, such as bethanechol.

In a third embodiment, the invention provides hydrophobic drug salts. In certain embodiments, the drug is a basic drug or a drug comprising a quaternized nitrogen atom and the salt comprises the protonated drug or quaternized drug as the cation and a hydrophobic anion, which is the conjugate base of a hydrophobic acid. In other embodiments, the drug is an acidic drug, and the salt comprises the conjugate base of the drug as the anion and a hydrophobic cation. In preferred embodiments, the salt has a cLog P of about 1 or greater when determined as disclosed herein.

The invention further includes pharmaceutical compositions comprising a salt of the invention and a pharmaceutically acceptable carrier or excipient.

In a fourth embodiment, the invention provides a composition comprising a polymeric film having embedded therein particles of a poorly soluble drug. In certain embodiments, the drug is present as the free acid or base. In other embodiments, the drug is present as a poorly water soluble salt. The drug particles are preferably substantially uniformly distributed through the film. In certain embodiments, the polymeric film is water soluble. In certain embodiments, the polymeric film has a melting point at or below physiological temperature, i.e., 37° C. In certain embodiments, the polymeric film is bioerodible or bioresorbable.

The invention further provides methods of use of the compositions disclosed herein for the delivery of a drug to a subject.

In a fifth embodiment, the invention provides a composition comprising particles of a poorly soluble drug and a wetting agent. In certain embodiments, the drug is present as a neutral compound, such as a free acid or base. In other embodiments, the drug is present as a salt. The drug particles are preferably suspended in a liquid vehicle in which at least one wetting excipient is dissolved. In certain embodiments, the liquid vehicle is aqueous.

The invention further provides methods of use of the compositions disclosed herein for the delivery of a drug to a subject.

In a sixth embodiment, the invention provides a formulation of a hydrophobic drug or a drug salt, such as a caine salt, incorporated into a rate controlling delivery tube for the purposes of sustained release of the drug. These tubes can be applied to the tissue directly or incorporated into dressings, bandages, creams, ointments, gels and lotions to provide for the extended release of an agent, such as anesthetic agent, preferably a caine, over many days. The rate of drug release is determined by the diameter of the tubes containing the drug salt and the inherent solubility of the salt itself. The duration of drug release is determined by the length of the tube.

### BRIEF DESCRIPTION OF THE DRAWINGS

FIG. 1 is an illustration of a polymeric tube delivery device of the invention.

FIG. 2 is an illustration of a wound dressing comprising polymeric delivery devices.

FIG. 3 is a graph of theoretical drug release over time as a function of the drug surface area for a 5 cm² dressing.

FIG. 4 is a graph of plasma lidocaine concentration (relative to Cmax) over time for the pharmacokinetic study described in Example 4.

FIG. 5 is a graph of plasma lidocaine concentration (ng/mL) over time for the pharmacokinetic study described in Example 4.

### DETAILED DESCRIPTION OF THE INVENTION

### I. Hydrophobic Drug Salts

In first embodiment, the invention provides acid addition salts of a monobasic or polybasic therapeutic agent wherein the acid is represented by Formula 1 or Formula 2:

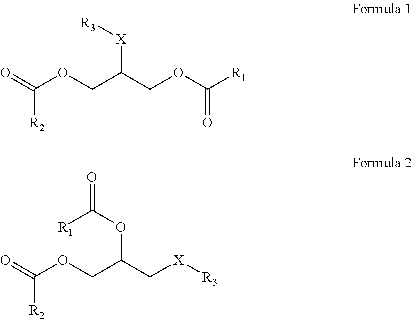

wherein R₁ and R₂ are each independently optionally substituted C₁-C₂₄-alkyl, optionally substituted C₂-C₂₄-alkenyl, optionally substituted C₂-C₂₄-alkynyl, optionally substituted C₃-C₁₂-cycloalkyl, or optionally substituted aryl; X is O or absent and R₃ is SO₃H, P(O)(OR₄)OH or C(O)OH; or X is O and R₃ is —CH₂C(O)OH, —CH₂C(O)SH or CH₂C(S)SH; or X is absent and R₃ is C(O)SH or C(S)SH. R₄ is hydrogen; optionally substituted alkyl, preferably optionally substituted C₁-C₆-alkyl; optionally substituted C₃-C₁₂-cycloalkyl; optionally substituted alkenyl, preferably optionally substituted C₂-C₆-alkenyl; optionally substituted alkynyl, preferably optionally substituted C₂-C₆-alkynyl; or optionally substituted aryl. R₄ is preferably hydrogen or C₁-C₆-alkyl and more preferably hydrogen. When the basic therapeutic agent, represented by B, is monobasic, the acid addition salt of the invention can be represented by BH⁺A⁻, where BH⁺ is the protonated, cationic form of the basic therapeutic agent form and A- is the conjugate base of the acid of Formula I or II. When the basic therapeutic agent, is monobasic and the basic functional group is a quaternary ammonium group, the acid addition salt of the invention can be represented by BA. The invention also provides pharmaceutical compositions comprising an acid addition salt of the invention and a pharmaceutically acceptable carrier or excipient.

In certain embodiments, one or both of R₁ and R₂ are independently an optionally substituted C₆-C₂₄-alkyl, -alkenyl or -alkynyl group, an optionally substituted C₆-C₁₂-cycloalkyl group or an optionally substituted phenyl group. In another embodiment, the number of carbon atoms in R₁ and R₂ combined is at least 10, 12, 14 or 16.

In certain embodiments, at least one of R₁ and R₂ is an optionally substituted aryl or aryl-C₁-C₆-alkyl group. Preferably, the aryl group is a phenyl, biphenyl, naphthyl, indenyl, indenyl, anthracenyl or phenanthryl group. More preferably, the aryl group is a phenyl group.

When the aryl group is substituted, preferred substituents include alkyl and halogen. In one embodiment, the optionally substituted aryl group is represented by

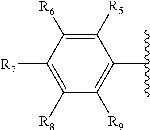

wherein R₅, R₆, R₇, R₈ and R₉ are each independently hydrogen, halogen, C₁-C₁₂-alkyl or halo-C₁-C₁₂-alkyl.

In certain embodiments, at least one of R₅, R₆, R₇, R₈ and R₉ is not hydrogen. In certain embodiments, at least one of R₅, R₆, R₇, R₈ and R₉ is alkyl. Preferred alkyl groups are C₁-C₁₂ alkyl groups, including C₃ to C₁₂ alkyl groups and C₄ to C₁₂-alkyl groups. Suitable alkyl groups include methyl, ethyl, n-propyl, i-propyl, n-butyl, sec-butyl, isobutyl, t-butyl, n-pentyl, pent-2-yl, pent-3-yl, 3-methylbutyl, 3-methylbut-2-yl, neopentyl, n-hexyl, hex-2-yl, hex-3-yl, 4-methylpentyl, 4-methylpent-2-yl, 3,3-dimethylbutyl, and 3,3-dimethylbut-2-yl. In certain embodiments, the alkyl group is methyl or an n-C₂-C₁₂-alkyl, and more preferably an n-C₃-C₁₂-alkyl, an n-C₃-C₁₀-alkyl, or an n-C₃-C₅-alkyl.

In certain embodiments, at least one of R₅, R₆, R₇, R₈ and R₉ is halogen. Preferred halogens include fluorine, chlorine and bromine. In certain embodiments, two, three, four or five of R₅ to R₉ are halogen. In embodiments in which two or more of R₅ to R₉ are halogen, the halogens can be the same or different. In certain embodiments, at least two of R₁ to R₅ is halogen and the halogens are the same. In certain embodiments, each of R₅ to R₉ is independently a halogen. In this embodiment, R₅ to R₆ are preferably the same halogen. In certain embodiments, the aryl group is selected from pentafluorophenyl, pentachlorophenyl and pentabromophenyl.

In certain embodiments, the aryl group is an optionally substituted polycyclic aryl group, preferably an optionally substituted biphenyl, naphthyl, anthracenyl, indenyl, indanyl or phenanthryl. Preferably, the number of substituents is 0 to 4. In preferred embodiments, the substituents are independently selected from alkyl, such as C₁-C₁₂-alkyyl; haloalkyl, such as halo-C₁-C₁₂-alkyl; and halogen, preferably fluorine, chlorine or bromine.

In certain embodiments, at least one of R₁ and R₂ is selected such that R₁C(O)OH or R₂C(O)OH is an acid which is Generally Recognized as Safe (“GRAS”) by the US Food and Drug Administration. Preferably, R₁ and R₂ are selected such that both R₁C(O)OH and R₂C(O)OH are GRAS acids. Examples of suitable GRAS acids include benzoic, capric, caproic, caprylic, lauric, myristic, palmitic, stearic, oleic, linoleic, linolenic, and arachidonic acids.

The conjugate bases of the acids of Formulas 1 and 2 are represented by Formulas 1 and 2 where R₃ is SO₃⁻, P(O)(OR₄)O⁻, —CH₂C(O), C(O)O⁻, —CH₂C(O)S, C(O)S⁻, CH₂C(S)S⁻ or C(S)S⁻ and R₁, R₂ and X have the meanings given for these variables above.

Suitable basic drugs are set forth as follows: Analgesics (opioids) and codeine derivatives such as morphine, benzylmorphine, propoxyphene, methadone, pentazocine, sufenatanil, alfentanil, fentanyl, pethidine, butorphanol, buprenorphine, diamorphine, dihydrocodeine, dypyrone, oxycodone, dipipanone, alphaprodine, levorphanol, dextromoramide, hydromorphone, nalbuphine, oxymorphone, hydrocodone, nalorphine (antagonist), naloxone (antagonist); Antimicrobials including quinolones such as norfloxacin, ciprofloxacin, lomefloxacin, balofioxacin, ofloxacin, sparfloxacin, tosufloxacin, temafloxacin, clinafloxacin, perfloxacin, tosufloxacin, enoxacin, amifloxacin, fleroxacin; Antimicrobials including aminoglycosides such as streptomycin, amikacin, gentamicin, tobramycin, neomycin, josamycin, spectinomycin, kanamycin, framycetin, paromomycin, sissomycin, viomycin; Glycopeptides such as vancomycin; Lincosamides such as clindamycin, lincomycin; Penicillins such as cephalosporins and cefepime, related β-lactams, cefmenoxime, cefotiam, cephalexin, bacampicillin, lenampicillin, pivampicillin, talampicillin; Macrolides such as erythromycin, oleandomycin; Tetracyclines such as tetracycline, minocycline, rolitetracycline, methacycline, meclocycline; Antimycobacterials such as isoniazid, pyrimethamine, ethambutol, Antivirals such as acyclovir, saquinavir, indinavir, ganciclovir, amantadine, moroxydine, rimantidine, famciclovir, zalcitabine, cidofovir, valacyclovir, lamivudine, nevirapine; Antiprotozoals such as metronidazole, temidazole, pentamidine, mepacrine, carnidazole, robenidine, emetine, dihydroemetine, halofuginone, homidium, melarsoprol; Antiseptics such as aminacrine; Antifungals such as ketoconazole, itraconazole, miconazole, econazole, clotrimazole, amphotericin B, butoconazole, chlormidazole, croconazole, diamthazole, fenticonazole, nystatin, cloconazole, econazole, miconazole, tioconazole; Anti-depressants such as clomipramine (all classes), lofepramine, phenelzine, tranylcypromine, dothiepin, nortryptaline, amitryptaline, imipramine, mianserin, maprotiline, desipramine, trazodone, fluoxetine, trimipramine, citalopram, doxepin, fluvoxamine, lofepramine, nomifensine, paroxetine, Anti-diabetics such as glipizide, metformin, phenformin; Anti-convulsants such as carbamazepine, ethosuxamide, diphenylhydantoin, phenytoin(—OH), primidone, methsuximide; Anticholinergics such as atropine (antimuscarinics), benztropine (all classes), scopolamine, homatropine, hyoscine, hyoscyamine, orphenadrine, pirenzipine,procyclidine, telenzipine, propantheline, dicyclomine, biperiden, trihexphenidyl, oxybutinin, benzhexol, biperiden, ipratropium, pipenzolate, mepenzolate, cyclopentolate; Anthelminitics such as albendazole, mebendazole, flubendazole, fenbendazole, pyrantel, ivermectin; Antigout such as allopurinol, colchicine; Antihistamines and chlorpheniramine phenothiazines such as dimenhydrinate (all classes), hydroxyzine, diphenhydramine, bromodiphenhydramine, astemizole, loratidine, acepromazine, thioridazine, brompheniramine, carbinoxamine, chlorcyclizine, chloropyramine, chlorphentermine, chlorprothixene, dexchlorpheniramine, antazoline, azatidine, azalastine, clemastine, clemizole, cyroheptadine, diphenylpyraline, doxylamine, flunarizine, mequitazine, meclozine, mepyramine, pheniramine, terfenadine, triprolidine, trimeprazine, ebastine, cinnarizine; Anti-migraines such as ergotamine, dihydroergotamine, methysergide, sumatriptan, naritriptan, almotriptan, zolmitriptan, rizatriptan, eletriptan, flumedroxone, pizotifen; Anti-tussives and dextromethorphan mucolytics such as pholcodeine, acetylcysteine, noscapine; Antineoplastics and azathiprine Immunosupressants such as methyluracil, fluorouracil, vincristine, vinblastine, melphalan, cyclophosphamide, aminoglutethimide, mercaptopurine, tamoxifen, chlorambucil, daunorubicin, mechlorethamine, doxorubicin; Anti-malarials such as quinine, chloroquine, pyrimethamine, amodiaquine, piperaquine, proguanil, chloroproguanil, mefloquine, primaquine, halofantrine; Anxiolytics, and Sedatives such as bromazepam; Hypnotics, and Antipsycotics such as nitrazepam, diazepam, oxazepam; Benzodiazepines such as clonazepam, chlorazepate, lorazepam, midazolam, triazolam, flunitrazepam; Butyrophenones such as droperidol, haloperidol; Barbiturates such as allobarbitone, aprobarbitone, phenobarbitone, amylobarbitone, barbitone, butobarbitone, zopiclone, hydroxyzine, buspirone, tandospirone, Bronchodilators such as theophylline; Cardiovascular Drugs including β-Blockers such as acebutatol, alprenolol, atenolol, labetalol, metopralol, nadolol, timolol, propanolol, pindolol, tolamolol, sotalol, oxprenolol, bunitrolol, carazolol, indenolol; Cardiovascular Drugs including Anti-arrythmics/cardiotonics such as disopyramide, cardiotonics, mexilitine, tocainide, aprindine, procainamide, quinidine, dobutamine; Cardiovascular Drugs including Ca channel blockers (all classes) including verapamil, diltiazem, amlodipine, felodipine, nicardipine, gallopamil, prenylamine; Cardiovascular Drugs including Antihypertensives/Vasodilators including diazoxide, guanethidine, clonidine, hydralizine, dihydralizine, minoxidil, prazosin, phenoxybenzamine, reserpine, phentolamine, perhexiline, indapamide, debrisoquine, bamethan, bethanidine, dobutamine, indoramin; Cardiovascular Drugs including Ace inhibitors captopril, enalapril, lisinopril, ramipril, imidapril; CNS stimulants/anorectics including methylphenidate, fenfluramine, amphetamine, methamphetamine, bemegride, caffeine, dexamphetamine, chlorphentamine, fencamfamine, prolintane; Diuretics such as furosemide, acetazolamide, amiloride, triampterene, bendrofluazide, chlorothiazide, chlorthalidone, cyclothiazide, hydroflumethiazide, hydrochlorothiazide, hydroflumethiazide; Gatrointestinal Agents including Motility enhancers, modulators and anti-emetics such as domperidone metoclopramide; cisapride, prochlorperazine, pirenzipine, cinitapride, cyclizine, chlorpromazine, prochloperazine, promethazine; Gatrointestinal Agents including Acid secretion modulators such as cimetidine, ranitidine, famotidine, omeprazole, nizatidine; Gatrointestinal Agents including Anti-diarrheals, including loperamide, diphenoxylate; Gatrointestinal Agents including emetics such as apomorphine; Muscle relaxants such as chlorzoxazon, rocuronium, suxamethonium, vecuronium, atracurium, fazadinium, doxacurium, mivacurium, pancuronium, tubocurarine, pipecurium, decamethonium, tizanidine, piridinol, succinylcholine, acetylcholine; Cholinergic Agents such as benzpyrinium, edrophonium, physostigmine, neostigmine, pyridostygmine; β-adrenergic agonists such as adrenaline ephedrine, pseudo-ephedrine, amidephrine, oxymetazoline, xylometazoline, terbutaline, salbutamol, salmeterol, phenylpropanolamine, cyclopentamine, phenylephrine, isoproterenol, fenoterol, xamoterol; Other CNS active agents such as dopamine, levodopa; Endocrine agents such as bromocriptine, propylthiouracil; Local anesthetics such as lidocaine (lignocaine), procaine, amethocaine, bupivacaine, butacaine, oxybuprocaine, mepivacaine, cocaine, prilocaine, amylocaine, chloroprocaine, cinchocaine, etidocaine, propoxycaine, tropacocaine, ropivacaine; Miscellaneous Mydriatics such as cyclopentolate, methazolamide, dorzolamide, acetazolamide, dynorphins, enkephalins, oxytocin and vasopressin. Additional basic therapeutic agents include naltrexone, varenicline, bacitracin, linezolid, daptomycin, granisetron, ondansetron, aripiprazole, risperidone, olanzapine, clozapine, thorazine, ipratropium, and bethanecol.

Preferably, the basic therapeutic agent is a local anesthetic including, but not limited to: lidocaine (lignocaine), procaine, amethocaine, bupivacaine, butacaine, oxybuprocaine, mepivacaine, cocaine, prilocaine, amylocaine, chloroprocaine, cinchocaine, etidocaine, propoxycaine, tropacocaine, and ropivacaine. Preferably the basic therapeutic agent is lidocaine, bupivacaine or ropivacaine.

Preferred acids of the invention and their conjugate bases are represented by Formulas 3-17 and 3A-17A below wherein R₁ and R₂ are each independently optionally substituted C₁-C₂₄-alkyl, optionally substituted C₂-C₂₄-alkenyl, optionally substituted C₂-C₂₄-alkynyl, optionally substituted C₃-C₁₂-cycloalkyl and optionally substituted aryl:

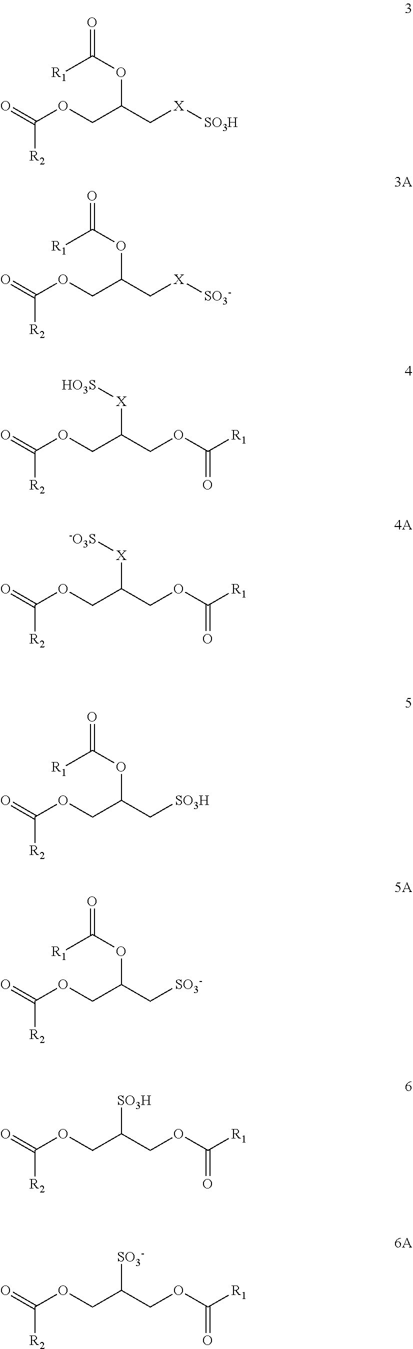

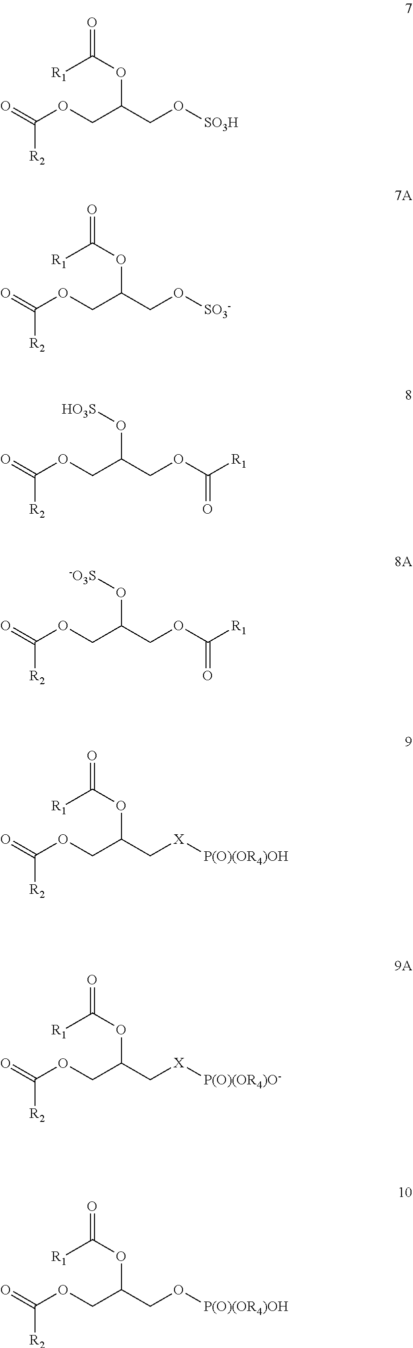

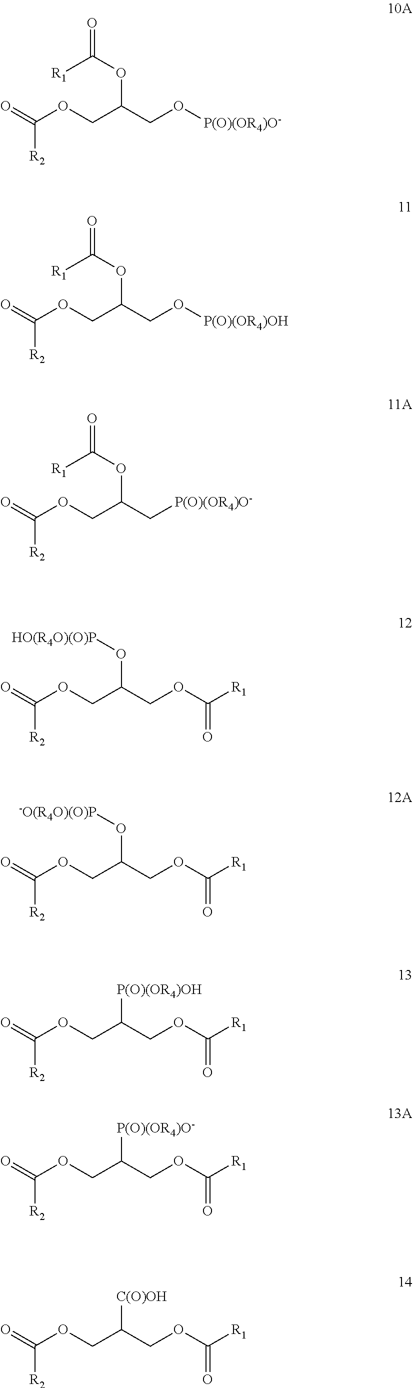

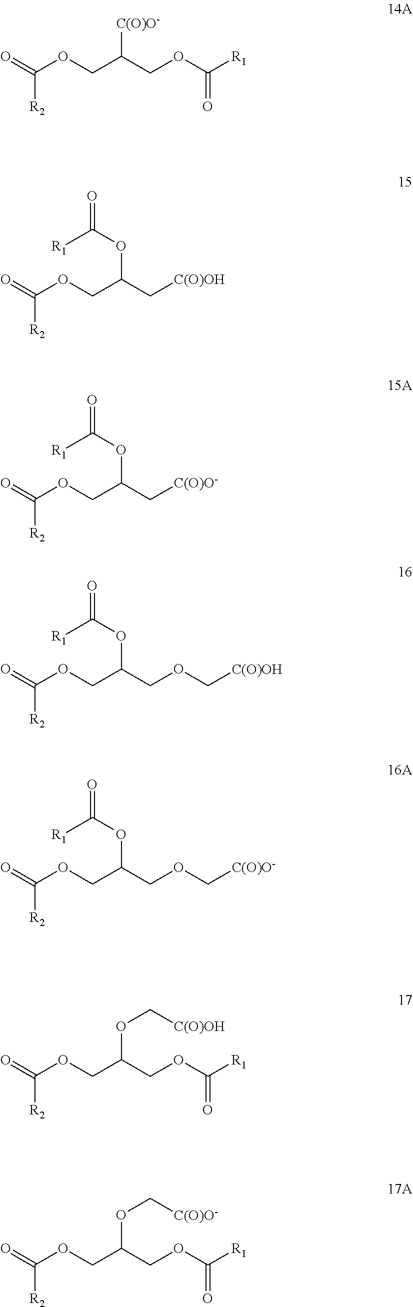

In certain embodiments, at least one of R₁ and R₂ of Formulas 1-6 and 1a-6a is selected from the moieties shown in Table 1. In certain embodiments R₁ and R₂ are the same or different and are selected from the moieties shown in Table 1.

TABLE 1

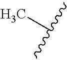

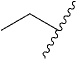 [SMILES: *CC]

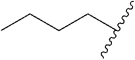 [SMILES: *CCCC]

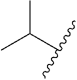 [SMILES: *C(C)C]

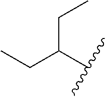 [SMILES: *C(CC)CC]

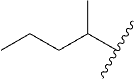 [SMILES: *C(C)CCC]

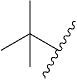 [SMILES: *C(C)(C)C]

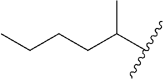 [SMILES: *C(C)CCCC]

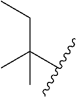 [SMILES: *C(C)(C)CC]

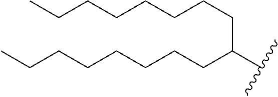 [SMILES: *C(CCCCCCCC)CCCCCCCC]

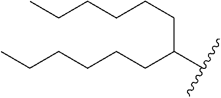 [SMILES: *C(CCCCCC)CCCCCC]

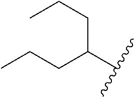 [SMILES: *C(CCC)CCC]

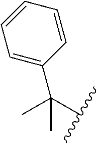 [SMILES: *C(C)(C)c1ccccc1]

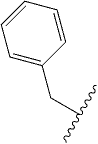 [SMILES: *Cc1ccccc1]

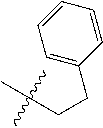 [SMILES: *CCc1ccccc1]

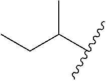 [SMILES: *C(C)CC]

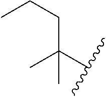 [SMILES: *C(C)(C)CCC]

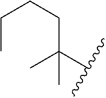 [SMILES: *C(C)(C)CCCC]

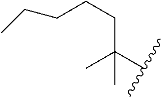 [SMILES: *C(C)(C)CCCCC]

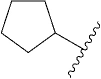 [SMILES: *C1CCCC1]

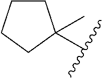 [SMILES: *C1(C)CCCC1]

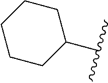 [SMILES: *C1CCCCC1]

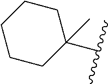 [SMILES: *C1(C)CCCCC1]

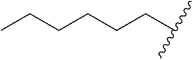 [SMILES: *CCCCCC]

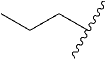 [SMILES: *CCC]

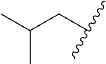 [SMILES: *CC(C)C]

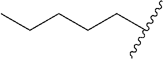 [SMILES: *CCCCC]

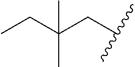 [SMILES: *CC(C)(C)CC]

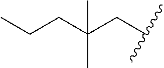 [SMILES: *CC(C)(C)CCC]

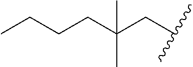 [SMILES: *CC(C)(C)CCCC]

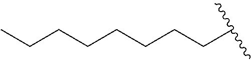 [SMILES: *CCCCCCCC]

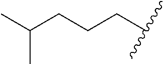 [SMILES: *CCCC(C)C]

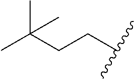 [SMILES: *CCC(C)(C)C]

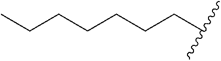 [SMILES: *CCCCCCC]

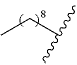

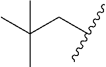 [SMILES: *CC(C)(C)C]

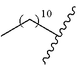

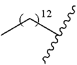

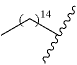

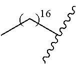

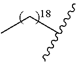

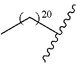

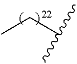

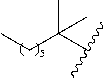

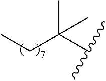

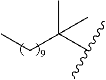

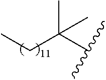

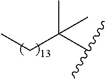

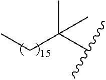

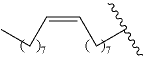

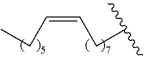

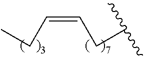

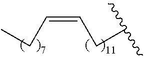

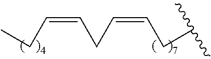

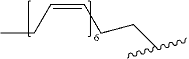

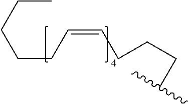

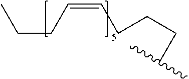

The acids of the invention represented by any of the foregoing formulas can exist as two, enantiomers exclusive of any asymmetric carbon atoms in R₁ and R₂. The acid addition salts of the invention can include a conjugate base of any of these acids as a single enantiomer, as a racemic mixture or as a mixture which has an excess of one enantiomer, for example, an enantiomeric excess of about 100 to about 99%.

The acid addition salts of basic therapeutic agents in accordance with the present invention provide, among other advantages, sustained or extended therapeutic levels of the therapeutic compound following administration. Sustained release may be due to several factors including, but not limited to, the decreased solubility of the acid addition salt relative to the parent drug, resulting in more gradual dissolution.

The choice of R₁ and R₂ moieties in Formulas 1-17 and 3A-17A can be used to selectively control the hydrophobicity and aqueous solubility of the resulting salt of any given basic therapeutic agent and thereby control the release rate of the drug.

In a preferred embodiment, a compound of the invention provides sustained delivery of the parent drug over hours, days, weeks or months when administered, for example, topically, orally or parenterally, to a subject. For example, when delivered parenterally, the compounds can provide sustained delivery of the drug for up to 1, 7, 15, 30, 60, 75 or 90 days or longer. Without being bound by theory, it is believed that the compounds of the invention form an insoluble depot upon parenteral administration, for example subcutaneous, intramuscular or intraperitoneal injection.

In certain embodiments, the salts of the invention are provided in the form of particles.

Preferably, the invention provides anesthetic particles for the treatment of pain due to an injury, particularly a wound, where the particles comprise as their major ingredient a salt BH⁺A⁻, where B is the free base of a local anesthetic, and is preferably a member of the caine family, BH+ is the cationic protonated form of B, and A⁻ is represented by any of Formulas 3A-17A. Preferably A⁻ is represented by one of Formulas 5A to 8A. Local anesthetics of the “caine” family are weak bases. (by “caine” is intended anesthetics that end in the suffix “caine”, which in certain embodiments include an amino acid amide or ester). One of the classes of caine anesthetics are amine bases and also include an aromatic ring, for example, a meta-xylyl group, and an amide or ester functionality. The aromatic group with the other entities results in hydrophobicity, so that the members of the class are frequently employed as their hydrochloride salts to allow for water solubility. Examples of such anesthetics of the caine family include lidocaine (lignocaine), procaine, bupivacaine, ropivacaine, butacaine, oxybuprocaine, mepivacaine, prilocaine, amylocaine, chloroprocaine, etidocaine, propoxycaine and tropacocaine. Caines of particular interest are lidocaine, bupivacaine and ropivacaine.

In the acids of the Formulas 1 to 17 that form the caine salts of particular interest, R₁ and R₂ will together preferably include a total of about 6 to 30 carbon atoms, more usually about 6 to 24, frequently about 8 to 24, particularly about 12 to 24 carbon atoms.

In certain embodiments, the caine salts of the invention are provided in the form of particles. In certain embodiments, the particles consist of one or more caine salts of the invention or consist essentially of one or more caine salts. In certain embodiments, the particles can have a 1:1 equivalent ratio of the anesthetic to the acid or one of the components may be in excess, usually not more than about 5-fold excess, generally up to about 0.5, or up to about a 0.2, equivalent excess of either of the components of the salt may be present. In certain embodiments, the particles include excess acid. By having excess acid, the release rate of the caine from the salt may be diminished by virtue of the common ion effect, where the dissolution of the excess acid will act to slow or retard the dissolution rate of the caine salt compound in the particles. In other embodiments, the particles include an acid addition caine salt, as described herein, and a second caine salt, such as a more soluble caine salt, for example, the caine hydrochloride salt. In another embodiment, a composition is provided comprising particles of the acid addition caine salt of the invention and particles of a more soluble caine salt, such as the caine hydrochloride salt.

The particles can further comprise two or more acid addition caine salts, differing in either or both of the caine agent and the acid. For example, the particles can comprise salts of the caine with two or more acids of Formulas 1 to 15 which differ in hydrophobicity. By using different acid addition salts, the rate of release of the anesthetic can be modulated, with acids with smaller R₁ and R₂ groups the smaller carbon acids usually providing for more rapid release. The composition may be a mixture of different sized particles, usually comprising not more than two different distributions, where each of the different distributions has at least about 75% of the weight of the particles within 50%, more usually within 25%, of the median weight. The median weights of the two differently sized compositions will usually differ by at least about 25%, more usually at least about 50% and there may be a two-fold difference or greater. In this way both composition and particle size can be varied to provide the optimum release profile for the particular application for the subject compositions.

In one embodiment, the composition comprises particles of a caine salt of the invention and a substantially soluble salt of the caine or a different caine. The soluble caine salt can be in a solid form, for example, in the form of particles, or in solution. In one embodiment, the particles of the caine salt of the invention are suspended in a solution comprising the soluble caine salt. The solution can be an aqueous solution or a solution of a pharmaceutically acceptable hydrophilic organic solvent. The soluble caine salt is preferably the hydrochloride, hydrobromide, acetic acid or nitric acid salt, preferably the hydrochloride salt. For example, the composition can comprise a salt of lidocaine, bupivacaine or ropivacaine with an acid of Formula 1 or Formula 2 and a soluble salt of one of these caines, such as lidocaine hydrochloride, bupivacaine hydrochloride or ropivacaine hydrochloride. Preferably, the same caine is present in both salts. Such compositions provide both a rapid onset of action due to the soluble salt and sustained action due to the caine salt of an acid of Formula I or Formula II.

The particles can further comprise one or more pharmaceutically acceptable excipients or additives, such as surfactants, polymers and salts. Preferably, the particles do not include a matrix, such as polymer matrix, which prolongs release of the caine anesthetic.

The size distribution of a particle composition of the caine salts of the invention will generally have at least about 50 weight % within 75%, more usually within 50%, and desirably within 25% of the median size. The median size will generally range from about 1 to about 2000 m, 5 to about 1500 μm, 5 to about 1200 μm, 50 to about 2000 m, more usually from about 50 to 1500 m, desirably from about 100 m to 1200 m. Individual compositions of interest have median sizes of about 1 to 25 m; 5 to 100 m; 100 to 200 m, 300 to 500 m, 500 to 750 am, 600 to 700 m and 750 to 1200 m. In one embodiment, the median size of the particles is about 625 to 675 am, or about 650 am.

Depending upon the manner in which the particles are made, they can comprise less than about 2, more usually less than about 1, weight % of the solvent used in their preparation, and preferably undetectable amounts.

The acid addition salts of local anesthetics of the invention are particularly useful for the treatment of pain. In certain embodiments, the pain is due to a wound, such as a wound due to trauma or surgery. In one embodiment, the salts are useful for the topical treatment of wounds, for example, surface wounds resulting from trauma or surgery. In treating the wound, the particles can be administered directly into the wound bed and onto the tissue for an open wound, for example. The particles can be administered by spraying, coating, painting, injecting, irrigating, adhered to a substrate, which substrate is placed in the wound, or the like. Spraying may be employed for administration of the particles with or without a vehicle, using a pharmacologically acceptable propellant. Air may be pumped to disseminate the particles.

Suitable topical vehicles, vehicles for aerosols and other components for use with the caine acid addition salts of the present invention are well known in the art. These vehicles may contain a number of different ingredients depending upon the nature of the vehicle, the nature of the wound, the manner of administration, and the like. The vehicles will provide for a convenient method of administration to the wound, while not adversely affecting the controlled release of the anesthetic from the particles.

Most common propellants are mixtures of volatile hydrocarbons, typically propane, n-butane and isobutane, or hydrofluoroalkanes (HFA): either HFA 134a (1,1,1,2-tetrafluoroethane) or HFA 227 (1,1,1,2,3,3,3-heptafluoropropane) or combinations of the two or compressed gases such as nitrogen, carbon dioxide, air and the like. One may also use a simple air brush means of dispensing the particles where there is literally no solvent but air is drawn and used to dispense the particles.

Liquid media used for dispersing the particles are preferably highly volatile or miscible with the aqueous environment of the wound and rapidly evaporate or dissipate under the conditions of administration. The liquids will for the most part be non-solvents for the anesthetic salt, although there may be minimal solubility. Such vehicles may include non-solvent liquid media that include water, mixtures of water and organic solvents and mixtures of organic solvents. Other additives may include protein-based materials such as collagen and gelatin; silicone-based materials; stabilizing and suspending agents; emulsifying agents; and other vehicle components that are suitable for administration to the skin, as well as mixtures of these components and those otherwise known in the art. The vehicle can further include components adapted to improve the stability or effectiveness of the applied formulation, such as preservatives, antioxidants, and skin penetration enhancers. Examples of such components are described in the following reference works hereby incorporated by reference: Martindale—The Extra Pharmacopoeia (Pharmaceutical Press, London 1993) and Martin (ed.), Remington's Pharmaceutical Sciences.

The choice of a suitable vehicle will depend on the particular physical form and mode of delivery that the formulation is to achieve. Examples of suitable forms include liquids; solids and semisolids such as gels, foams, pastes, creams, ointments, powders and the like; colloidal drug delivery systems, for example, liposomes, microemulsions, microparticles, or other forms.

The topical formulations of the caine addition salts of the invention can be prepared in a variety of physical forms. For example, solid particles, pastes, creams, lotions, gels, and liquids are all contemplated by the present invention. A difference between these forms is their physical appearance and viscosity, which can be governed by the presence and amount of emulsifiers and viscosity adjusters present in the formulation. Particular topical formulations can often be prepared in a variety of these forms. Solids are generally firm and will usually be in particulate form; solids optionally can contain liquids, emulsifiers, moisturizers, emollients, fragrances, dyes/colorants, preservatives and other active ingredients that increase or enhance the efficacy of the final product. Creams and lotions are often similar to one another, differing mainly in their viscosity; both lotions and creams may be opaque, translucent or clear and often contain emulsifiers, solvents, and viscosity adjusting agents, as well as moisturizers, emollients, fragrances, dyes/colorants, preservatives and other active ingredients that increase or enhance the efficacy of the final product. Gels can be prepared with a range of viscosities, from thick or high viscosity to thin or low viscosity. These formulations, like those of lotions and creams may also contain liquids, emulsifiers, moisturizers, emollients, fragrances, dyes/colorants, preservatives and other ingredients that increase or enhance the efficacy of the final product. Liquids are thinner than creams, lotions, or gels and often do not contain emulsifiers.

Suitable emulsifiers for use in the caine addition salt formulations of the present invention include, but are not limited to ionic emulsifiers, behentirmonium methosulfate, cetearyl alcohol, non-ionic emulsifiers like polyoxyethylene oleyl ether, PEG-40 sterate, ceteareth-12, ceteareth-20, ceteareth-30, ceteareth alcohol, PEG-100 stearate, glyceryl stearate, or combinations or mixtures thereof.

Suitable viscosity adjusting agents for use in the caine acid addition salt formulations of the present invention include, but are not limited to protective colloids or non-ionic gums such as hydroxyethylcellulose, xanthan gum, magnesium aluminum silicate, silica, microcrystalline wax, beeswax, paraffin, and cetyl palmitate, or combinations or mixtures thereof.

Suitable liquids for use in the caine acid addition salt formulations of the present invention will be selected to be non-irritating and include, but are not limited to water, propylene glycol, polyethylene glycols, polypropylene glycols and mixtures thereof. Not more than about 10 weight %, usually not more than 5 weight %, of the anesthetic salt will be soluble in the medium; preferably the anesthetic salt will be insoluble in the medium.

Suitable surfactants for use in the caine acid addition formulations of the present invention include, but are not limited to nonionic surfactants. For example, dimethicone copolyol, polyethylene glycols, including higher PEGs, such as PEG200, polysorbate 20, polysorbate 40, polysorbate 60, polysorbate 80, lauramide DEA, cocamide DEA, and cocamide MEA, are contemplated for use with the formulations of the present invention. In addition, combinations or mixtures of these surfactants can be used in the formulations of the present invention.

Suitable preservatives for use in the caine acid addition salt formulations of the present invention include, but are not limited to antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. In addition, combinations or mixtures of these preservatives can be used in the formulations of the present invention.

Suitable moisturizers for use in the caine acid addition formulations of the present invention include, but are not limited to lactic acid and other hydroxy acids and their salts, glycerin, propylene glycol, and butylene glycol. Suitable emollients include lanolin alcohol, lanolin, lanolin derivatives, cholesterol, petrolatum, isostearyl neopentanoate and mineral oils. In addition, combinations or mixtures of these moisturizers and emollients can be used in the formulations of the present invention.

Other suitable additional ingredients that may be included in the caine acid addition salt formulation of the present invention include, but are not limited to, abrasives, absorbents, anticaking agents, anti-foaming agents, anti-static agents, astringents, binders/excipients, buffering agents, chelating agents, film forming agents, conditioning agents, opacifying agents, pH adjusters and protectants. Examples of each of these ingredients in topical product formulations, can be found in publications by The Cosmetic, Toiletry, and Fragrance Association (CTFA). See, e.g., CTFA Cosmetic Ingredient Handbook, 2^{nd} edition, eds. John A. Wenninger and G. N. McEwen, Jr. (CTFA, 1992).

In many instances it may be desirable that the health care professional administering the particle formulation is able to insure uniform coverage or otherwise be able to see what areas have been covered and how extensively the particle formulation has been distributed. Therefore, one may include a detectable composition with the particles so that they can be visualized. This may include colored compounds or dyes, fluorescent compounds and even luminescent compounds. The dyes should be highly colored and visible in the presence of blood, while the fluorescent compounds should fluoresce under ultra-violet light. See, for example, Richard P. Haugland; Molecular Probes-Handbook of Fluorescent Probes and Research Chemicals; 5th Edition 1992-94; Molecular Probes, Inc.

The particles will typically be at least about 1 weight %, usually at least 2 weight %, and up to 100 weight % of the non-volatile portion of the composition. Where the particles are dispersed in a vehicle, the weight % of the particles will generally be in the range of about 1-75 weight %, more usually about 1-50 weight %. The minor ingredients except for the medium will generally range from about 0.01 weight % to about 10 weight %, the amount generally being conventional for the purpose of the ingredient. Where the particles are sprayed as an aerosol, generally the particles will be present in the range of about 1 to 99 weight % of the composition.

Depending upon the need and the nature of the composition, the composition may be sprayed, wiped, smeared, painted, transferred from a template onto or proximal to the wound or may be made into a patch where the composition will be separate from or part of the adhesive. Alternatively, topically the composition may be applied to the wound and a dressing or other protective layer added to prevent contamination and abrasion. In some situations, the composition may be injected, particularly where a minimally invasive surgical technique is employed and the rate of transdermal transport is insufficient to provide the pain relief required. Not more than one application should be required per 6 hours, usually per half-day, and times between applications may vary from 6 hours to 7 days, usually 12 hours to 4 days, where frequently by 7 days, further treatment will not be required. During this time a therapeutically effective amount of the caine will be released from the particles.

The amount of the anesthetic salt applied to the wound area will be a therapeutically effective amount to minimize pain to a level that the patient can tolerate and preferably substantially eliminate any sense of pain. The amount of pain will usually vary with time, so that the amount of anesthetic that will be required can be diminished over time. Therefore, the profile of anesthetic release from the salt can be a diminishing amount of anesthetic being released over time. Conveniently, there may be an initial large release, less than about 30%, usually less than about 25%, of the total amount of anesthetic followed by a decreasing release over time at a lower amount at a therapeutic level. The large initial release coincides with the high levels of pain in the early post-operative period. After the initial release, generally not more than 60 weight %, more usually not more than about 50 weight %, will be released in 24 hours, where the pain alleviation is to occur over generally greater than two days, with diminishing percentages as the time for relief is extended.

Once the particles have been prepared, irrespective of the method employed in their preparation, the particles are sized and fractioned typically by sieving operations, although other methods may be employed. To control particle distribution and particle size a typical sieving operation would employ at least 2 sieves of the appropriate size. The larger sieve size would allow for the rejection of particles larger than the specified maximum while the lower sieve size would serve to retain the particles of the specified size. The selection of the sieves determines the particle size distribution. Using this approach one can also prepare multimodal distributions to obtain different release profiles of drug. Nominal particle size and particle size distribution is determined by an instrument such as a Coulter LS13 on suspensions of the microparticles.

Drug dissolution kinetics is evaluated using an LC method employing an infinite sink concept. A known amount of microparticles are suspended in a defined volume of a suitable test medium, for example a phosphate buffer solution containing 1% Tween 80, meant to simulate in vivo release kinetics. The suspension of microparticles is kept at a constant temperature, typically 37° C., for a period of time, for example, about 12 hours, with constant agitation. The particles are removed from the solution by filtration and re-suspended in another fresh amount of the test media. The original solution is assayed for the amount of drug product in solution by an appropriate quantitative method, typically an LC method employing UV detection or MS.

If fluorescent or colored microparticles are desired the procedure for making the microparticle is followed, however, for a fluorescent product a compound such as fluorescein is added to the mixture before the precipitation or preparation of the microparticle is attempted. If a colored product is required a food safe dye such as FD&C Blue No 1 or Blue No 2 is used.

Drug product of the appropriate size is combined with other agents that may be appropriate to provide free flowing stable microparticles and added to an appropriate aerosol container. The aerosol container is subsequently pressurized with a high purity propellant and sealed under pressure with the appropriate spray nozzle to provide the spray pattern desired and in some cases to provide a metered dose of the drug. Alternatively, the drug product can be suspended into a PBS solution or other suitable vehicle just prior to application to the wound. The product is distributed over the wound by spraying using a variety of possible propulsion systems e.g. an air brush type of system, pump sprayer system, etc., whereby drug product suspended in the PBS is aspirated through a tube using the Venturi concept with a propellant container.

The acids of the invention and the acid addition salts thereof can be prepared by methods known in the art. For example, an acid addition salt of a basic drug in accordance with the invention may be prepared by any conventional means, including precipitation of the salt from solution, spray drying a solution of the salt, reaction of the drug and acid in solution and removal of solvent, or fusion of the free base of the drug with the acid. In one embodiment the free base of the drug compound is combined with the acid in a suitable solvent, such as water or a polar organic solvent. Alternatively, a salt of the drug, such as the hydrochloride salt, is reacted with a salt of the acid, for example, the sodium salt, in water or a polar organic solvent. In either case, the desired salt can either spontaneously precipitate upon formation or be induced to precipitate by adding a suitable cosolvent and/or concentrating the solution. In certain embodiments, the free base of the drug is combined with the acid in the absence of solvent, resulting in the formation of the desired salt.

The acids and salts of the invention can be prepared, for example, via the methods shown in Schemes 1 and 2 below.

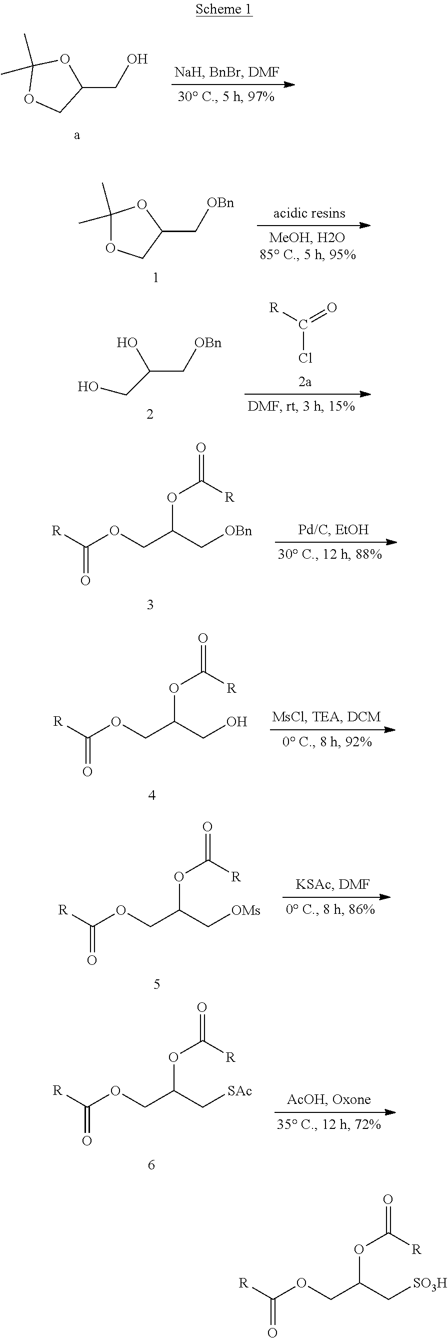

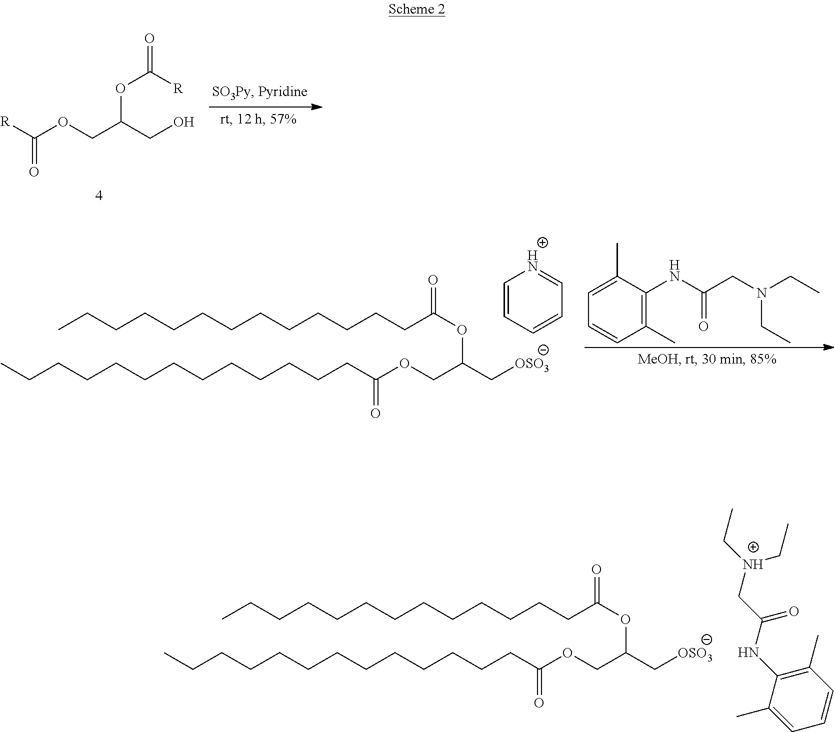

Each R═CH₃(CH₂)₁₂—

### II. Hydrophobic Drug Salts

In a second embodiment, the present invention provides hydrophobic pharmaceutical salts. In one embodiment, the hydrophobic pharmaceutical salt is an acid addition salt of a basic drug wherein the acid is an organic acid which comprises at least six carbon atoms. The organic acid can be, for example, a carboxylic acid, a sulfonic acid, a phosphonic acid, a sulfuric acid ester or a phosphoric acid ester, each having six or more carbon atoms. Preferably, the acid comprises an optionally substituted alkyl, alkenyl, alkynyl or aryl group having at least six carbon atoms, for example from six to about 30 carbon atoms. In certain embodiments, the acid is an optionally substituted C₆-C₃₀-alkylcarboxylic acid, an optionally substituted C₆-C₃₀-alkylsulfonic acid, an optionally substituted C₆-C₃₀-alkylphosphonic acid, an optionally substituted sulfuric acid C₆-C₃₀-alkyl ester or an optionally substituted phosphoric acid C₆-C₃₀-alkyl ester. In certain embodiments, the acid is an optionally substituted C₆-C₃₀-alkenylcarboxylic acid, an optionally substituted C₆-C₃₀-alkenylsulfonic acid, an optionally substituted C₆-C₃₀-alkenylphosphonic acid, an optionally substituted sulfuric acid C₆-C₃₀-alkenyl ester or an optionally substituted phosphoric acid C₆-C₃-alkenyl ester. In yet other embodiments, the acid is an optionally substituted C₆-C₃₀-alkynylcarboxylic acid, an optionally substituted C₆-C₃₀-alkynylsulfonic acid, an optionally substituted C₆-C₃-alkynylphosphonic acid, an optionally substituted sulfuric acid C₆-C₃₀-alkynyl ester or an optionally substituted phosphoric acid C₆-C₃-alkynyl ester. In further embodiments, the acid is an optionally substituted C₆-C₁₄-arylcarboxylic acid, an optionally substituted C₆-C₁₄-arylsulfonic acid, an optionally substituted C₆-C₁₄-arylphosphonic acid, an optionally substituted sulfuric acid C₆-C₁₄-aryl ester or an optionally substituted phosphoric acid C₆-C₁₄-aryl ester.

In certain embodiments, the drug has a basic functional group, for example, a primary, secondary or tertiary amine, which is protonated in the salt. In other embodiments, the drug has a quaternary ammonium group.

The drug can be an addiction treatment, such as naltrexone, varenicline or buprenorphine; an antibiotic, such as neomycin, vancomycin, linezolid or clindamycin; an anti-emetic, such as granisetron or ondansetron; a psychoactive agent, such as aripiprazole, respiridone, olanzapine, clozapine, haloperidol or chlorpromazine; a bronchodilator, such as iprotropium or tiotropium; or a urinary drug, such as bethanechol.

In another embodiment, the pharmaceutical salt is a salt of an acidic drug with a hydrophobic cation, for example, an ammonium or phosphonium cation having at least four carbon atoms, preferably at least six carbon atoms. In another embodiment, the cation is an optionally quaternized nitrogen containing heteroaromatic compound, for example an optionally substituted pyridinium or quinolinium cation wherein the nitrogen atom is protonated or quaternized.

The ammonium cation can be a primary, secondary, tertiary or quaternary ammonium group, and can comprise at least one optionally substituted aryl or alkyl group, provided that the total number of carbon atoms is at least four and preferably at least six.

The nitrogen containing heteraromatic compound preferably comprises a nitrogen-containing six membered ring. Suitable heteroaromatic groups include optionally substituted pyridine, quinoline and isoquinoline. In the salt, the nitrogen atom can be protonated or quaternized, for example, the nitrogen atom can be alkylated.

The acidic drug is preferably bacitracin, ertapenem, meropenem, imipenem or epoprostanol.

Suitable substituents for alkyl, alkenyl and alkynyl groups, include, but are not limited to halogen, preferably fluorine, chlorine or bromine, and optionally substituted aryl groups, preferably optionally substituted C₆-C₁₄-aryl groups.

Suitable substituents for aryl and heteroaryl groups include, but are not limited to, halogen, such as fluorine, chlorine or bromine, optionally substituted C₁-C₂₄-alkyl, C₂-C₂₄-alkenyl or C₂-C₂₄-alkynyl.

The invention also provides a pharmaceutical composition comprising a hydrophobic salt of the invention and a pharmaceutically acceptable excipient or carrier.

The invention further includes methods of treating a disease or disorder in a subject in need thereof, comprising administering to the subject an effective amount of a hydrophobic salt of the invention.

The drug salts in accordance with the present invention provide, among other advantages, sustained or extended therapeutic levels of the therapeutic compound following administration. “Sustained release” typically refers to shifting absorption toward slow pseudo first-order release kinetics or to other release profiles based upon how particles may aggregate in vivo. Sustained release may be due to several factors including, but not limited to, the decreased solubility of the drug salt relative to the parent drug.

The choice of cation or anion in the drug salt can be used to selectively control the hydrophobicity and aqueous solubility of the resulting salt and thereby control the release rate of the drug.

In a preferred embodiment, a compound of the invention provides sustained delivery of the parent drug over hours, days, weeks or months when administered, for example, topically, orally or parenterally, to a subject. For example, when delivered parenterally, the compounds can provide sustained delivery of the drug for up to 1, 7, 15, 30, 60, 75 or 90 days or longer. Without being bound by theory, it is believed that the salts of the invention form an insoluble depot upon parenteral administration, for example by subcutaneous, intramuscular or intraperitoneal injection.

In certain embodiments, the salts of the invention are provided in the form of particles.

The particles can further comprise one or more pharmaceutically acceptable excipients or additives, such as surfactants, polymers and salts. Preferably, the particles do not include a matrix, such as polymer matrix, which prolongs release of the drug.

The size distribution of a particle composition of the salts of the invention will generally have at least about 50 weight % within 75%, more usually within 50%, and desirably within 25% of the median size. The median size will generally range from about 1 to about 2000 m, more usually from about 5 to 1500 m, desirably from about 5 m to 1200 m. Individual compositions of interest have median sizes of about 1 to 25 m; 5 to 100 m; 100 to 200 m, 300 to 500 m, 500 to 750 m, 600 to 700 m and 750 to 1200 m. In one embodiment, the median size of the particles is about 625 to 675 m, or about 650 m.

Depending upon the manner in which the particles are made, they can comprise less than about 2, more usually less than about 1, weight % of the solvent used in their preparation, and preferably undetectable amounts.

Suitable topical vehicles, vehicles for aerosols and other components for use with the salts of the present invention are well known in the art. These vehicles may contain a number of different ingredients depending upon the nature of the vehicle, the nature of the wound, the manner of administration, and the like. The vehicles will provide for a convenient method of administration to the wound, while not adversely affecting the controlled release of the anesthetic from the particles.

Most common propellants are mixtures of volatile hydrocarbons, typically propane, n-butane and isobutane, or hydrofluoroalkanes (IFA): either HIFA 134a (1,1,1,2,-tetrafluoroethane) or HIFA 227 (1,1,1,2,3,3,3-heptafluoropropane) or combinations of the two or compressed gases such as nitrogen, carbon dioxide, air and the like. One may also use a simple air brush means of dispensing the particles where there is literally no solvent but air is drawn and used to dispense the particles.

Liquid media used for dispersing the particles are preferably highly volatile or miscible with the aqueous environment of the wound and rapidly evaporate or dissipate under the conditions of administration. The liquids will for the most part be non-solvents for the anesthetic salt, although there may be minimal solubility. Such vehicles may include non-solvent liquid media that include water, mixtures of water and organic solvents and mixtures of organic solvents. Other additives may include protein-based materials such as collagen and gelatin; silicone-based materials; stabilizing and suspending agents; emulsifying agents; and other vehicle components that are suitable for administration to the skin, as well as mixtures of these components and those otherwise known in the art. The vehicle can further include components adapted to improve the stability or effectiveness of the applied formulation, such as preservatives, antioxidants, and skin penetration enhancers. Examples of such components are described in the following reference works hereby incorporated by reference: Martindale, The Extra Pharmacopoeia (Pharmaceutical Press, London 1993) and Martin (ed.), Remington's Pharmaceutical Sciences.

The choice of a suitable vehicle will depend on the particular physical form and mode of delivery that the formulation is to achieve. Examples of suitable forms include liquids; solids and semisolids such as gels, foams, pastes, creams, ointments, powders and the like; colloidal drug delivery systems, for example, liposomes, microemulsions, microparticles, or other forms.

The topical formulations of the salts of the invention can be prepared in a variety of physical forms. For example, solid particles, pastes, creams, lotions, gels, and liquids are all contemplated by the present invention. A difference between these forms is their physical appearance and viscosity, which can be governed by the presence and amount of emulsifiers and viscosity adjusters present in the formulation. Particular topical formulations can often be prepared in a variety of these forms. Solids are generally firm and will usually be in particulate form; solids optionally can contain liquids, emulsifiers, moisturizers, emollients, fragrances, dyes/colorants, preservatives and other active ingredients that increase or enhance the efficacy of the final product. Creams and lotions are often similar to one another, differing mainly in their viscosity; both lotions and creams may be opaque, translucent or clear and often contain emulsifiers, solvents, and viscosity adjusting agents, as well as moisturizers, emollients, fragrances, dyes/colorants, preservatives and other active ingredients that increase or enhance the efficacy of the final product. Gels can be prepared with a range of viscosities, from thick or high viscosity to thin or low viscosity. These formulations, like those of lotions and creams may also contain liquids, emulsifiers, moisturizers, emollients, fragrances, dyes/colorants, preservatives and other ingredients that increase or enhance the efficacy of the final product. Liquids are thinner than creams, lotions, or gels and often do not contain emulsifiers.

Suitable emulsifiers for use in the salt formulations of the present invention include, but are not limited to ionic emulsifiers, behentirmonium methosulfate, cetearyl alcohol, non-ionic emulsifiers like polyoxyethylene oleyl ether, PEG-40 stearate, ceteareth-12, ceteareth-20, ceteareth-30, ceteareth alcohol, PEG-100 stearate, glyceryl stearate, or combinations or mixtures thereof.

Suitable viscosity adjusting agents for use in the salt formulations of the present invention include, but are not limited to protective colloids or non-ionic gums such as hydroxyethylcellulose, xanthan gum, magnesium aluminum silicate, silica, microcrystalline wax, beeswax, paraffin, and cetyl palmitate, or combinations or mixtures thereof.

Suitable liquids for use in the salt formulations of the present invention will be selected to be non-irritating and include, but are not limited to water, propylene glycol, polyethylene glycols, polypropylene glycols and mixtures thereof. Not more than about 10 weight %, usually not more than 5 weight %, of the anesthetic salt will be soluble in the medium; preferably the anesthetic salt will be insoluble in the medium.

Suitable surfactants for use in the salt formulations of the present invention include, but are not limited to nonionic surfactants. For example, dimethicone copolyol, polyethylene glycols, including higher PEGs, such as PEG200, polysorbate 20, polysorbate 40, polysorbate 60, polysorbate 80, lauramide DEA, cocamide DEA, and cocamide MEA, are contemplated for use with the formulations of the present invention. In addition, combinations or mixtures of these surfactants can be used in the formulations of the present invention.

Suitable preservatives for use in the salt formulations of the present invention include, but are not limited to antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. In addition, combinations or mixtures of these preservatives can be used in the formulations of the present invention.

Suitable moisturizers for use in the salt formulations of the present invention include, but are not limited to lactic acid and other hydroxy acids and their salts, glycerin, propylene glycol, and butylene glycol. Suitable emollients include lanolin alcohol, lanolin, lanolin derivatives, cholesterol, petrolatum, lipids, phospolipids, isostearyl neopentanoate and mineral oils. In addition, combinations or mixtures of these moisturizers and emollients can be used in the formulations of the present invention.

Other suitable additional ingredients that may be included in the salt formulation of the present invention include, but are not limited to, abrasives, absorbents, anticaking agents, anti-foaming agents, anti-static agents, astringents, binders/excipients, buffering agents, chelating agents, film forming agents, conditioning agents, opacifying agents, pH adjusters and protectants. Examples of each of these ingredients in topical product formulations, can be found in publications by The Cosmetic, Toiletry, and Fragrance Association (CTFA). See, e.g., CTFA Cosmetic Ingredient Handbook, 2^{nd} edition, eds. John A. Wenninger and G. N.

McEwen, Jr. (CTFA, 1992).

In many instances it may be desirable that the health care professional administering the particle formulation is able to insure uniform coverage or otherwise be able to see what areas have been covered and how extensively the particle formulation has been distributed.

Therefore, one may include a detectable composition with the particles so that they can be visualized. This may include colored compounds or dyes, fluorescent compounds and even luminescent compounds. The dyes should be highly colored and visible in the presence of blood, while the fluorescent compounds should fluoresce under ultra-violet light. See, for example, Richard P. Haugland; Molecular Probes-Handbook of Fluorescent Probes and Research Chemicals; 5th Edition 1992-94; Molecular Probes, Inc.

The particles will typically be at least about 1 weight %, usually at least 2 weight %, and up to 100 weight % of the non-volatile portion of the composition. Where the particles are dispersed in a vehicle, the weight % of the particles will generally be in the range of about 1-75 weight %, more usually about 1-50 weight %. The minor ingredients except for the medium will generally range from about 0.01 weight % to about 10 weight %, the amount generally being conventional for the purpose of the ingredient. Where the particles are sprayed as an aerosol, generally the particles will be present in the range of about 1 to 99 weight % of the composition.

Once the particles have been prepared, irrespective of the method employed in their preparation, the particles can be sized and fractioned typically by sieving operations, although other methods may be employed. To control particle distribution and particle size a typical sieving operation would employ at least 2 sieves of the appropriate size. The larger sieve size would allow for the rejection of particles larger than the specified maximum while the lower sieve size would serve to retain the particles of the specified size. The selection of the sieves determines the particle size distribution. Using this approach one can also prepare multimodal distributions to obtain different release profiles of drug. Nominal particle size and particle size distribution is determined by an instrument such as a Coulter LS13 on suspensions of the microparticles.

Drug dissolution kinetics is evaluated using an LC method employing an infinite sink concept. A known amount of microparticles are suspended in a defined volume of a suitable test medium, for example a phosphate buffer solution containing 1% Tween 80, meant to simulate in vivo release kinetics. The suspension of microparticles is kept at a constant temperature, typically 37° C., for a period of time, for example, about 12 hours, with constant agitation. The particles are removed from the solution by filtration and re-suspended in another fresh amount of the test media. The original solution is assayed for the amount of drug product in solution by an appropriate quantitative method, typically an LC method employing UV detection or MS.

If fluorescent or colored microparticles are desired the procedure for making the microparticle is followed, however, for a fluorescent product a compound such as fluorescein is added to the mixture before the precipitation or preparation of the microparticle is attempted. If a colored product is required a food safe dye such as FD&C Blue No. 1 or Blue No. 2 is used.

Drug product of the appropriate size is combined with other agents that may be appropriate to provide free flowing stable microparticles and added to an appropriate aerosol container. The aerosol container is subsequently pressurized with a high purity propellant and sealed under pressure with the appropriate spray nozzle to provide the spray pattern desired and in some cases to provide a metered dose of the drug. Alternatively, the drug product can be suspended into a PBS solution or other suitable vehicle just prior to application to the wound.

The product is distributed over the wound by spraying using a variety of possible propulsion systems e.g., an air brush type of system, pump sprayer system, etc., whereby drug product suspended in the PBS is aspirated through a tube using the Venturi concept with a propellant container.

The salts of the invention can be prepared by methods known in the art. For example, an acid of a basic or acidic drug in accordance with the invention may be prepared by any conventional means, including precipitation of the salt from solution, spray drying a solution of the salt, reaction of the drug and acid in solution and removal of solvent, or fusion of the free base of the drug with the acid. In one embodiment the free base of the drug compound is combined with the acid in a suitable solvent, such as water or a polar organic solvent. Alternatively, a salt of the drug, such as the hydrochloride salt, is reacted with a salt of the acid, for example, the sodium salt, in water or a polar organic solvent. In either case, the desired salt can either spontaneously precipitate upon formation or be induced to precipitate by adding a suitable cosolvent and/or concentrating the solution. In certain embodiments, the free base of the drug is combined with the acid in the absence of solvent, resulting in the formation of the desired salt.

### III. Hydrophobic Drug Salts

In a third embodiment, the invention provides hydrophobic drug salts having a cLog P of about 1 or greater when determined as disclosed herein. In certain embodiments, the drug is a basic drug or a drug comprising a quaternized nitrogen atom and the salt comprises the protonated drug or quaternized drug as the cation and a hydrophobic anion, which is the conjugate base of a hydrophobic acid. In other embodiments, the drug is an acidic drug, and the salt comprises the conjugate base of the drug as the anion and a hydrophobic cation.

In one embodiment, the drug salts of this embodiment of the invention comprise as the cation a protonated basic drug or a quaternized drug. The anion is the conjugate base of a hydrophobic acid. In certain embodiments, the anion is the conjugate base of an organic acid which comprises at least six carbon atoms. The organic acid can be, for example, a carboxylic acid, a sulfonic acid, a phosphonic acid, a sulfuric acid ester or a phosphoric acid ester, each having six or more carbon atoms. Preferably, the acid comprises an optionally substituted alkyl, alkenyl, alkynyl or aryl group having at least six carbon atoms, for example from six to about 30 carbon atoms. In certain embodiments, the acid is an optionally substituted C₆-C₃₀-alkylcarboxylic acid, an optionally substituted C₆-C₃₀-alkylsulfonic acid, an optionally substituted C₆-C₃₀-alkylphosphonic acid, an optionally substituted sulfuric acid C₆-C₃-alkyl ester or an optionally substituted phosphoric acid C₆-C₃-alkyl ester. In certain embodiments, the acid is an optionally substituted C₆-C₃₀-alkenylcarboxylic acid, an optionally substituted C₆-C₃₀-alkenylsulfonic acid, an optionally substituted C₆-C₃₀-alkenylphosphonic acid, an optionally substituted sulfuric acid C₆-C₃₀-alkenyl ester or an optionally substituted phosphoric acid C₆-C₃₀-alkenyl ester. In yet other embodiments, the acid is an optionally substituted C₆-C₃₀-alkynylcarboxylic acid, an optionally substituted C₆-C₃₀-alkynylsulfonic acid, an optionally substituted C₆-C₃₀-alkynylphosphonic acid, an optionally substituted sulfuric acid C₆-C₃₀-alkynyl ester or an optionally substituted phosphoric acid C₆-C₃₀-alkynyl ester. In further embodiments, the acid is an optionally substituted C₆-C₁₄-arylcarboxylic acid, an optionally substituted C₆-C₁₄-arylsulfonic acid, an optionally substituted C₆-C₁₄-arylphosphonic acid, an optionally substituted sulfuric acid C₆-C₁₄-aryl ester or an optionally substituted phosphoric acid C₆-C₁₄-aryl ester.

In certain embodiments, the anion is the conjugate base of a fatty acid, such as an acid selected from C₄-C₂₄-alkyl-C(O)OH or C₄-C₂₄-alkyl-S(O)₂OH, preferably a C₆-C₂₄-alkyl-C(O)OH or a C₆-C₂₄-alkyl-S(O)₂OH.

Suitable basic drugs are set forth as follows: Analgesics (opioids) and codeine derivatives such as morphine, benzylmorphine, propoxyphene, methadone, pentazocine, sufenatanil, alfentanil, fentanyl, pethidine, butorphanol, buprenorphine, diamorphine, dihydrocodeine, dypyrone, oxycodone, dipipanone, alphaprodine, levorphanol, dextromoramide, hydromorphone, nalbuphine, oxymorphone, hydrocodone, nalorphine (antagonist), naloxone (antagonist); Antimicrobials including quinolones such as norfloxacin, ciprofloxacin, lomefloxacin, balofioxacin, ofloxacin, sparfloxacin, tosufloxacin, temafloxacin, clinafloxacin, perfloxacin, tosufloxacin, enoxacin, amifloxacin, fleroxacin; Antimicrobials including aminoglycosides such as streptomycin, amikacin, gentamicin, tobramycin, neomycin, josamycin, spectinomycin, kanamycin, framycetin, paromomycin, sissomycin, viomycin; Glycopeptides such as vancomycin; Lincosamides such as clindamycin, lincomycin; Penicillins such as cephalosporins and cefepime, related β-lactams, cefmenoxime, cefotiam, cephalexin, bacampicillin, lenampicillin, pivampicillin, talampicillin; Macrolides such as erythromycin, oleandomycin; Tetracyclines such as tetracycline, minocycline, rolitetracycline, methacycline, meclocycline; Antimycobacterials such as isoniazid, pyrimethamine, ethambutol, Antivirals such as acyclovir, saquinavir, indinavir, ganciclovir, amantadine, moroxydine, rimantidine, famciclovir, zalcitabine, cidofovir, valacyclovir, lamivudine, nevirapine; Antiprotozoals such as metronidazole, temidazole, pentamidine, mepacrine, carnidazole, robenidine, emetine, dihydroemetine, halofuginone, homidium, melarsoprol; Antiseptics such as aminacrine; Antifungals such as ketoconazole, itraconazole, miconazole, econazole, clotrimazole, amphotericin B, butoconazole, chlormidazole, croconazole, diamthazole, fenticonazole, nystatin, cloconazole, econazole, miconazole, tioconazole; Anti-depressants such as clomipramine (all classes), lofepramine, phenelzine, tranylcypromine, dothiepin, nortryptaline, amitryptaline, imipramine, mianserin, maprotiline, desipramine, trazodone, fluoxetine, trimipramine, citalopram, doxepin, fluvoxamine, lofepramine, nomifensine, paroxetine, Anti-diabetics such as glipizide, metformin, phenformin; Anti-convulsants such as carbamazepine, ethosuxamide, diphenylhydantoin, phenytoin(—OH), primidone, methsuximide; Anticholinergics such as atropine (antimuscarinics), benztropine (all classes), scopolamine, homatropine, hyoscine, hyoscyamine, orphenadrine, pirenzipine, procyclidine, telenzipine, propantheline, dicyclomine, biperiden, trihexphenidyl, oxybutinin, benzhexol, biperiden, ipratropium, pipenzolate, mepenzolate, cyclopentolate; Anthelminitics such as albendazole, mebendazole, flubendazole, fenbendazole, pyrantel, ivermectin; Antigout such as allopurinol, colchicine; Antihistamines and chlorpheniramine phenothiazines such as dimenhydrinate (all classes), hydroxyzine, diphenhydramine, bromodiphenhydramine, astemizole, loratidine, acepromazine, thioridazine, brompheniramine, carbinoxamine, chlorcyclizine, chloropyramine, chlorphentermine, chlorprothixene, dexchlorpheniramine, antazoline, azatidine, azalastine, clemastine, clemizole, cyroheptadine, diphenylpyraline, doxylamine, flunarizine, mequitazine, meclozine, mepyramine, pheniramine, terfenadine, triprolidine, trimeprazine, ebastine, cinnarizine; Anti-migraines such as ergotamine, dihydroergotamine, methysergide, sumatriptan, naritriptan, almotriptan, zolmitriptan, rizatriptan, eletriptan, flumedroxone, pizotifen; Anti-tussives and dextromethorphan mucolytics such as pholcodeine, acetylcysteine, noscapine; Antineoplastics and azathiprine Immunosupressants such as methyluracil, fluorouracil, vincristine, vinblastine, melphalan, cyclophosphamide, aminoglutethimide, mercaptopurine, tamoxifen, chlorambucil, daunorubicin, mechlorethamine, doxorubicin; Anti-malarials such as quinine, chloroquine, pyrimethamine, amodiaquine, piperaquine, proguanil, chloroproguanil, mefloquine, primaquine, halofantrine; Anxiolytics, and Sedatives such as bromazepam; Hypnotics, and Antipsycotics such as nitrazepam, diazepam, oxazepam; Benzodiazepines such as clonazepam, chlorazepate, lorazepam, midazolam, triazolam, flunitrazepam; Butyrophenones such as droperidol, haloperidol; Barbiturates such as allobarbitone, aprobarbitone, phenobarbitone, amylobarbitone, barbitone, butobarbitone, zopiclone, hydroxyzine, buspirone, tandospirone, Bronchodilators such as theophylline; Cardiovascular Drugs including β-Blockers such as acebutatol, alprenolol, atenolol, labetalol, metopralol, nadolol, timolol, propanolol, pindolol, tolamolol, sotalol, oxprenolol, bunitrolol, carazolol, indenolol; Cardiovascular Drugs including Anti-arrythmics/cardiotonics such as disopyramide, cardiotonics, mexilitine, tocainide, aprindine, procainamide, quinidine, dobutamine; Cardiovascular Drugs including Ca channel blockers (all classes) including verapamil, diltiazem, amlodipine, felodipine, nicardipine, gallopamil, prenylamine; Cardiovascular Drugs including Antihypertensives/Vasodilators including diazoxide, guanethidine, clonidine, hydralizine, dihydralizine, minoxidil, prazosin, phenoxybenzamine, reserpine, phentolamine, perhexiline, indapamide, debrisoquine, bamethan, bethanidine, dobutamine, indoramin; Cardiovascular Drugs including Ace inhibitors captopril, enalapril, lisinopril, ramipril, imidapril; CNS stimulants/anorectics including methylphenidate, fenfluramine, amphetamine, methamphetamine, bemegride, caffeine, dexamphetamine, chlorphentamine, fencamfamine, prolintane; Diuretics such as furosemide, acetazolamide, amiloride, triampterene, bendrofluazide, chlorothiazide, chlorthalidone, cyclothiazide, hydroflumethiazide, hydrochlorothiazide, hydroflumethiazide; Gatrointestinal Agents including Motility enhancers, modulators and anti-emetics such as domperidone metoclopramide; cisapride, prochlorperazine, pirenzipine, cinitapride, cyclizine, chlorpromazine, prochloperazine, promethazine; Gatrointestinal Agents including Acid secretion modulators such as cimetidine, ranitidine, famotidine, omeprazole, nizatidine; Gatrointestinal Agents including Anti-diarrhealsincluding loperamide, diphenoxylate; Gatrointestinal Agents including emetics such as apomorphine; Muscle relaxants such as chlorzoxazon, rocuronium, suxamethonium, vecuronium, atracurium, fazadinium, doxacurium, mivacurium, pancuronium, tubocurarine, pipecurium, decamethonium, tizanidine, piridinol, succinylcholine, acetylcholine; Cholinergic Agents such as benzpyrinium, edrophonium, physostigmine, neostigmine, pyridostygmine; β-adrenergic agonists such as adrenaline ephedrine, pseudo-ephedrine, amidephrine, oxymetazoline, xylometazoline, terbutaline, salbutamol, salmeterol, phenylpropanolamine, cyclopentamine, phenylephrine, isoproterenol, fenoterol, xamoterol; Other CNS active agents such as dopamine, levodopa; Endocrine agents such as bromocriptine, propylthiouracil; Local anesthetics such as lidocaine (lignocaine), procaine, amethocaine, bupivacaine, butacaine, oxybuprocaine, mepivacaine, cocaine, prilocaine, amylocaine, chloroprocaine, cinchocaine, etidocaine, propoxycaine, tropacocaine, ropivacaine; Miscellaneous Mydriatics such as cyclopentolate, methazolamide, dorzolamide, acetazolamide, dynorphins, enkephalins, oxytocin and vasopressin. Additional basic therapeutic agents include naltrexone, varenicline, bacitracin, linezolid, daptomycin, granisetron, ondansetron, aripiprazole, risperidone, olanzapine, clozapine, thorazine, ipratropium, and bethanecol.

Preferably, the basic therapeutic agent is a local anesthetic such as, but not limited to: lidocaine (lignocaine), procaine, amethocaine, bupivacaine, butacaine, oxybuprocaine, mepivacaine, cocaine, prilocaine, amylocaine, chloroprocaine, cinchocaine, etidocaine, propoxycaine, tropacocaine, and ropivacaine. Preferably the basic therapeutic agent is lidocaine, bupivacaine or ropivacaine. Preferred acid addition salts of the invention include the the perfluoro-n-butane-1-sulfonate, perfluoro-n-pentane-1-sulfonate and perfluoro-n-hexane-1-sulfonate salts of lidocaine, bupivacaine and ropivacaine.

Suitable acidic drugs include, but are not limited to, meropenem, imipenem, ertapenem, prostacyclin, phenytoin, warfarin, tolbutamide, theophyllline, sulfapyridine, sulfadizine, salicyclic acid, propylthiouracil, phenobarbital, pentobarbital, peniciiamine, methyldopa, methotrexate, levodopa, ibuprofen, furosemide, ethacrynic acid, cephalexin, ciprofloxacin, chlorothiazide, aspirin, ampicillin, acetazolamide, and amoxicillin.

The compositions in accordance with the present invention provide, among other advantages, sustained or extended therapeutic levels of the drug following administration. Sustained release may be due to several factors including, but not limited to, the decreased solubility of the salt relative to the parent drug.

In a preferred embodiment, a composition of the invention provides sustained delivery of the drug over hours, days, weeks or months when administered, for example, topically, orally or parenterally, to a subject.

In certain embodiments, the conjugate base of a hydrophobic acid or the hydrophobic cation disclosed herein has relatively low surface activity or surfactancy. In certain embodiments, the conjugate base of the acids or the cations disclosed herein have a critical micelle concentration (“CMC”) in water at 1 atmosphere and 25° C. which is greater than 20 mM. In certain embodiments, the CMC is greater than 30 mM, 40 mM or 50 mM. In other embodiments, the CMC is greater than 70 mM, 90 mM, 100 mM, 125 mM, 150 mM, 175 mM, 200 mM or 225 mM.

The salts disclosed herein preferably have a Log P value of 1 or greater, for example, 2 or greater, 3 or greater, 4 or greater or 5 or greater, as calculated using ACD Labs software. This approach to calculating Log P employs a Classic model, which relies on the separation of the molecule in question into its constituent parts and summing those values as determined for sample compounds that have been tabulated from the literature. The cLog P values are determined in this approach for the combined drug and anion or cation, in which each species is considered in its neutral form. That is, an acid addition salt of a basic therapeutic agent is evaluated based on the neutral acid and the free base of the drug. Similarly, a salt of an acidic drug and a protonated cation is evaluated based on the acid form of the drug and the free base of the cation.

### IV. Compositions of Drug Salts

The invention further provides compositions of hydrophobic drugs and drug salts as set forth below in sections V, VI, VII and IX below. The drug salts of use in these compositions include salts of basic drugs with a hydrophobic anion and salts of acidic drugs with a hydrophobic cation. Such salts include those described in sections I-III above. Other hydrophobic drugs and drug salts which can used in the compositions of the invention include those described in U.S. Pat. No. RE46397, the contents of which are incorporated herein in their entirety.

The drug salts of the invention comprise as the cation a protonated basic drug or a quaternized drug. The anion is the conjugate base of a hydrophobic acid. In certain embodiments, the anion is the conjugate base of an organic acid which comprises at least six carbon atoms. The organic acid can be, for example, a carboxylic acid, a sulfonic acid, a phosphonic acid, a sulfuric acid ester or a phosphoric acid ester, each having six or more carbon atoms. Preferably, the acid comprises an optionally substituted alkyl, alkenyl, alkynyl or aryl group having at least six carbon atoms, for example from six to about 30 carbon atoms. In certain embodiments, the acid is an optionally substituted C₆-C₃₀-alkylcarboxylic acid, an optionally substituted C₆-C₃₀-alkylsulfonic acid, an optionally substituted C₆-C₃₀-alkylphosphonic acid, an optionally substituted sulfuric acid C₆-C₃₀-alkyl ester or an optionally substituted phosphoric acid C₆-C₃₀-alkyl ester. In certain embodiments, the acid is an optionally substituted C₆-C₃₀-alkenylcarboxylic acid, an optionally substituted C₆-C₃₀-alkenylsulfonic acid, an optionally substituted C₆-C₃₀-alkenylphosphonic acid, an optionally substituted sulfuric acid C₆-C₃-alkenyl ester or an optionally substituted phosphoric acid C₆-C₃₀-alkenyl ester. In yet other embodiments, the acid is an optionally substituted C₆-C₃₀-alkynylcarboxylic acid, an optionally substituted C₆-C₃₀-alkynylsulfonic acid, an optionally substituted C₆-C₃₀-alkynylphosphonic acid, an optionally substituted sulfuric acid C₆-C₃₀-alkynyl ester or an optionally substituted phosphoric acid C₆-C₃₀-alkynyl ester. In further embodiments, the acid is an optionally substituted C₆-C₁₄-arylcarboxylic acid, an optionally substituted C₆-C₁₄-arylsulfonic acid, an optionally substituted C₆-C₁₄-arylphosphonic acid, an optionally substituted sulfuric acid C₆-C₁₄-aryl ester or an optionally substituted phosphoric acid C₆-C₁₄-aryl ester.

In certain embodiments, the anion is the conjugate base of a fatty acid, such as an acid selected from C₄-C₂₄-alkyl-C(O)OH or C₄-C₂₄-alkyl-S(O)₂OH, preferably a C₆-C₂₄-alkyl-C(O)OH or a C₆-C₂₄-alkyl-S(O)₂OH.

In one embodiment, the poorly water soluble drug is an acid addition salt of a basic therapeutic agent, wherein the acid is represented by Formula I

R—X (I)

wherein R is a haloalkyl group and X is —SO₃H, C(O)OH or —P(O)(OH)(OR₁), wherein R₁ is hydrogen or C₁-C₆-alkyl. Preferably X is —SO₃H. The haloalkyl group can be straight chain or branched. Suitable haloalkyl groups include halo-n-propyl, halo-i-propyl, halo-n-butyl, halo-sec-butyl, halo-isobutyl, halo-t-butyl, halo-n-pentyl, halopent-2-yl, halopent-3-yl, halo-3-methylbutyl, halo-3-methylbut-2-yl, halo-neopentyl, halo-n-hexyl, halo-hex-2-yl, halo-hex-3-yl, halo-4-methylpentyl, halo-4-methylpent-2-yl, halo-3,3-dimethylbutyl, and halo-3,3-dimethylbut-2-yl. Preferably, the haloalkyl group is a halo-n-C₂-C₁₀-alkyl, and more preferably halo-n-C₃-C₆-alkyl. Most preferably the haloalkyl group is a fluoroalkyl group, such as fluoro-n-propyl, fluoro-n-butyl, fluoro-n-pentyl or fluoro-n-hexyl.

In preferred embodiments, R is a perhaloalkyl group. In certain embodiments, R is a perfluoroalkyl group or a perchloroalkyl group. Preferably R is a perhalo-C₂-C₁₀-alkyl group; more preferably a perhalo-C₃-C₆-alkyl group. The perhaloalkyl group can be straight chain or branched. Suitable perhaloalkyl groups include perhalo-n-propyl, perhalo-i-propyl, perhalo-n-butyl, perhalo-sec-butyl, perhalo-isobutyl, perhalo-t-butyl, perhalo-n-pentyl, perhalopent-2-yl, perhalopent-3-yl, perhalo-3-methylbutyl perhalo-3-methylbut-2-yl, perhalo-neopentyl, perhalo-n-hexyl, perhalo-hex-2-yl, perhalo-hex-3-yl, perhalo-4-methylpentyl, perhalo-4-methylpent-2-yl, perhalo-3,3-dimethylbutyl, and perhalo-3,3-dimethylbut-2-yl. Preferably, the perhaloalkyl group is a perhalo-n-C₂-C₁₀-alkyl, and more preferably perhalo-n-C₃-C₆-alkyl. Most preferably the perhaloalkyl group is a perchloroalkyl or perfluoroalkyl group, such as perchloro-n-propyl, perchloro-n-butyl, perchloro-n-pentyl, perchloro-n-hexyl, perfluoro-n-propyl, perfluoro-n-butyl, perfluoro-n-pentyl or perfluoro-n-hexyl.

In another embodiment, the acid is represented by Formula IV:

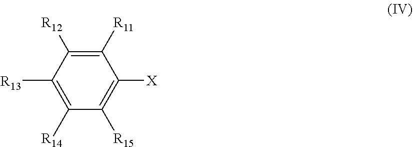

wherein R₁₁, R₁₂, R₁₃, R₁₄ and R₁₅ are each independently hydrogen, halogen, C₁-C₁₂-alkyl or halo-C₁-C₁₂-alkyl; and X is —SO₃H, —C(O)OH or —PO₃H; provided that at least one of R₁₁, R₁₂, R₁₃, R₁₄ and R₁₅ is is not hydrogen and further provided that the compounds of Formula I do not include 4-methylbenzenesulfonic acid.

In another embodiment, the invention provides an acid addition salt of a basic therapeutic agent wherein the acid is of Formula V,

Ar—X (V),

wherein Ar is an optionally substituted polycyclic aryl group and X is as defined above. Preferably the polycyclic aryl group is an optionally substituted biphenyl, naphthyl, anthracenyl, or indenyl. Preferably, the number of substituents is 0 to 4. In preferred embodiments, the substituents are independently selected from alkyl, haloalkyl and halogen.

In preferred embodiments of the compounds of Formula IV and Formula V, X is —SO₃H.

In certain embodiments of the compounds of Formula IV, at least one of R₁₁ to R₁₅ is a halogen. In certain embodiments, two, three, four or five of R₁₁ to R₁₅ are halogen. In compounds having more than one halogen, the halogens can be the same or different. In certain embodiments, at least two of R₁₁ to R₁₅ is halogen and the halogens are the same. Preferred halogens include fluorine, chlorine and bromine. In certain embodiments, each of R₁₁ to R₁₅ is independently a halogen. In this embodiment, R₁₁ to R₁₅ are preferably the same halogen. In certain embodiments,

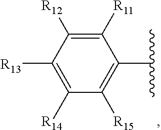

is pentafluorophenyl, pentachlorophenyl or pentabromophenyl.

In certain embodiments of the compounds of Formula IV, at least one of R₁₁ to R₁₅ is an alkyl group. In certain embodiments, two, three, four or five of R to R₁₅ are alkyl groups. In compounds having more than one alkyl groups, the alkyl groups can be the same or different. In certain embodiments, at least two of R₁₁ to R₁₅ are the same alkyl group.

In certain embodiments of the compounds of Formula IV, at least one of R₁₁ to R₁₅ is a haloalkyl group. In certain embodiments, two, three, four or five of R₁₁ to R₁₅ are haloalkyl groups. In compounds having more than one haloalkyl groups, the haloalkyl groups can be the same or different. In certain embodiments, at least two of R₁₁ to R₁₅ are the same haloalkyl group.

In certain embodiments of the compounds of Formula IV, at least one of R₁₁ to R₁₅ is a halogen, preferably bromo, chloro or fluoro, and at least one of the others is an alkyl or haloalkyl group, such as methyl or trifluoromethyl.

In certain embodiments of the compounds of Formula IV, at least one of R₁₁ to R₁₅ is hydrogen. Preferably, at least two or at least three of R₁₁ to R₁₅ are hydrogen. In certain embodiments, four of R₁₁ to R₁₅ are hydrogen.

In one embodiment, the compound of Formula IV is 3,5-dibromo-4-methylbenzenesulfonic acid.

In certain embodiments, the acid addition salt of the invention is represented by Formula VI:

B(H)ₘ₊ₙ^{(m+n)+}WₘYₙ (VI)

where B is a basic drug, W is the conjugate base of a hydrophobic acid, such as an acid of any one of Formulas I to V; Y is a pharmaceutically acceptable monoanion other than W, m+n is the number of basic groups on B, provided that m is at least 1. Preferably m+n is 1, 2, or 3.

Preferred acid addition salts are represented by Formula VII,

B(H)ₘ^{m+W}ₘ (VII)

where m is the number of protonated basic groups on B, preferably 1, 2 or 3.

In particularly preferred embodiments, B is a monobasic drug (i.e., m is 1 and n is 0) and the acid addition salt of the invention is represented by Formula VIII,

BH⁺W (VIII).

In another embodiment, the drug is an acidic therapeutic agent and the salt comprises the conjugate base of the drug and a hydrophobic cation. The hydrophobic cation can be, for example, an ammonium or phosphonium cation having at least four carbon atoms, preferably at least six carbon atoms. In another embodiment, the cation is an optionally quaternized nitrogen containing heteroaromatic compound, for example an optionally substituted pyridinium or quinolinium cation wherein the nitrogen atom is protonated or quaternized.

The ammonium cation can be a primary, secondary, tertiary or quaternary ammonium group, and can comprise at least one optionally substituted aryl or alkyl group, provided that the total number of carbon atoms is at least four and preferably at least six.

The nitrogen containing heteroaromatic compound preferably comprises a nitrogen-containing six membered ring. Suitable heteroaromatic groups include optionally substituted pyridine, quinoline and isoquinoline. In the salt, the nitrogen atom can be protonated or quaternized, for example, the nitrogen atom can be alkylated.

Suitable substituents for alkyl, alkenyl and alkynyl groups, include, but are not limited to halogen, preferably fluorine, chlorine or bromine, and optionally substituted aryl groups, preferably optionally substituted C₆-C₁₄-aryl groups.

Suitable substituents for aryl and heteroaryl groups include, but are not limited to, halogen, such as fluorine, chlorine or bromine, optionally substituted C₁-C₂₄-alkyl, C₂-C₂₄-alkenyl or C₂-C₂₄-alkynyl.

In one embodiment, the hydrophobic cation is represented by Formula IX,

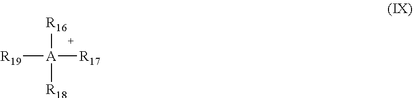

wherein, A is nitrogen, R₁₆ is

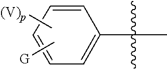

wherein G is C₁-C₁₂-alkyl, C₂-C₁₂-alkenyl or C₂-C₁₂ alkynyl; each V is independently halogen or C₁-C₆-alkyl and p is 0 to 4; or R₁ is C₄-C₁₂-alkyl; and R₁₇-R₁₉ are each independently hydrogen or C₁-C₆-alkyl.

Alternatively, A is nitrogen, R₁₆ is C₄-C₁₂-alkyl and R₁₇, R₁₈, and R₁₉ are each independently hydrogen or C₁-C₆-alkyl.

Alternatively, A is nitrogen, R₁₆ is C₁-C₆-alkyl and R₁₇, R₁₈, and R₁₉ are each independently hydrogen or C₁-C₆-alkyl, provided that at least one of R₁₇, R₁₈ and R₁₉ is not hydrogen.

Alternatively, A is phosphorus, and R₁₆ to R₁₉ are each independently selected from: phenyl having 0 to 5 substituents selected from halogen and C₁-C₆-alkyl; C₁-C₆-alkyl and halo-C₁-C₆-alkyl.

In certain embodiments, the cation of Formula IX is represented by Formula X,

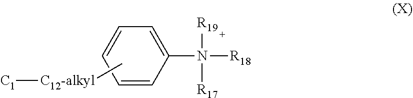

wherein R₁₇, R₁₈, and R₁₉ are as previously defined. Preferably, R₁₇, R₁₈, and R₁₉ are each independently methyl or hydrogen. In one embodiment, R₁₇, R₁₈, and R₁₉ are each hydrogen. In another embodiment, R₁₇, R₁₈, and R₁₉ are each methyl. In certain embodiments, the C₁-C₁₂-alkyl group is at the para position.

In certain embodiments, the cation of Formula IX is represented by Formula XI,

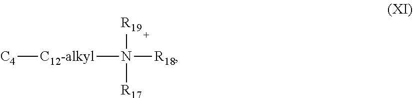

wherein R₂, R₃, and R₄ are as previously defined. Preferably, R₂, R₃, an R₄ are each independently methyl or hydrogen. In one embodiment, R₂, R₃, and R₄ are each hydrogen. In another embodiment, R₂, R₃, and R₄ are each methyl.

In certain embodiments, the cation of Formula I is represented by Formula XII,

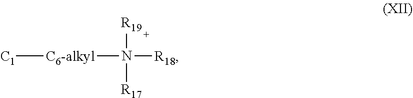

wherein R₂, R₃, and R₄ are as previously defined, provided that at least one of R₂, R₃, and R₄ is not hydrogen. Preferably, R₂ is methyl and R₃ and R₄ are each independently methyl or hydrogen. In another embodiment, R₂, R₃, and R₄ are each methyl.

In certain embodiments, the cation of Formula I is represented by Formula XIII,

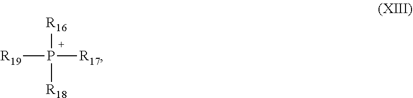

wherein R₁₆ to R₁₉ are each independently selected from: phenyl having 0 to 5 substituents selected from halogen and C₁-C₆-alkyl; C₁-C₆-alkyl and halo-C₁-C₆-alkyl. In certain embodiments, the cation of Formula V is tetraphenylphosphonium or tetramethylphosphonium.

In certain embodiments, the acid addition salt of the invention is represented by Formula XIV:

Y⁺B⁻ (XIV)

where B⁻ is the conjugate base of an acidic drug and Y+ is a hydrophobic cation, for example, a cation of any one of Formulas IX to XIII.

It is to be understood that a quaternary ammonium functional group carries a positive charge without protonation. Thus, in a basic drug which has such a group, the overall positive charge on the drug compound will be greater than the number of protonated sites. For example, in salts of Formula V in which the basic drug has a single basic functional group which is a quaternary ammonium group, there is no additional proton present and the formula is B RW.

Suitable basic drugs are set forth as follows: Analgesics (opioids) and codeine derivatives such as morphine, benzylmorphine, propoxyphene, methadone, pentazocine, sufenatanil, alfentanil, fentanyl, pethidine, butorphanol, buprenorphine, diamorphine, dihydrocodeine, dypyrone, oxycodone, dipipanone, alphaprodine, levorphanol, dextromoramide, hydromorphone, nalbuphine, oxymorphone, hydrocodone, nalorphine (antagonist), naloxone (antagonist); Antimicrobials including quinolones such as norfloxacin, ciprofloxacin, lomefloxacin, balofioxacin, ofloxacin, sparfloxacin, tosufloxacin, temafloxacin, clinafloxacin, perfloxacin, tosufloxacin, enoxacin, amifloxacin, fleroxacin; Antimicrobials including aminoglycosides such as streptomycin, amikacin, gentamicin, tobramycin, neomycin, josamycin, spectinomycin, kanamycin, framycetin, paromomycin, sissomycin, viomycin; Glycopeptides such as vancomycin; Lincosamides such as clindamycin, lincomycin; Penicillins such as cephalosporins and cefepime, related β-lactams, cefmenoxime, cefotiam, cephalexin, bacampicillin, lenampicillin, pivampicillin, talampicillin; Macrolides such as erythromycin, oleandomycin; Tetracyclines such as tetracycline, minocycline, rolitetracycline, methacycline, meclocycline; Antimycobacterials such as isoniazid, pyrimethamine, ethambutol, Antivirals such as acyclovir, saquinavir, indinavir, ganciclovir, amantadine, moroxydine, rimantidine, famciclovir, zalcitabine, cidofovir, valacyclovir, lamivudine, nevirapine; Antiprotozoals such as metronidazole, temidazole, pentamidine, mepacrine, carnidazole, robenidine, emetine, dihydroemetine, halofuginone, homidium, melarsoprol; Antiseptics such as aminacrine; Antifungals such as ketoconazole, itraconazole, miconazole, econazole, clotrimazole, amphotericin B, butoconazole, chlormidazole, croconazole, diamthazole, fenticonazole, nystatin, cloconazole, econazole, miconazole, tioconazole; Anti-depressants such as clomipramine (all classes), lofepramine, phenelzine, tranylcypromine, dothiepin, nortryptaline, amitryptaline, imipramine, mianserin, maprotiline, desipramine, trazodone, fluoxetine, trimipramine, citalopram, doxepin, fluvoxamine, lofepramine, nomifensine, paroxetine, Anti-diabetics such as glipizide, metformin, phenformin; Anti-convulsants such as carbamazepine, ethosuxamide, diphenylhydantoin, phenytoin(—OH), primidone, methsuximide; Anticholinergics such as atropine (antimuscarinics), benztropine (all classes), scopolamine, homatropine, hyoscine, hyoscyamine, orphenadrine, pirenzipine, procyclidine, telenzipine, propantheline, dicyclomine, biperiden, trihexphenidyl, oxybutinin, benzhexol, biperiden, ipratropium, pipenzolate, mepenzolate, cyclopentolate; Anthelminitics such as albendazole, mebendazole, flubendazole, fenbendazole, pyrantel, ivermectin; Antigout such as allopurinol, colchicine; Antihistamines and chlorpheniramine phenothiazines such as dimenhydrinate (all classes), hydroxyzine, diphenhydramine, bromodiphenhydramine, astemizole, loratidine, acepromazine, thioridazine, brompheniramine, carbinoxamine, chlorcyclizine, chloropyramine, chlorphentermine, chlorprothixene, dexchlorpheniramine, antazoline, azatidine, azalastine, clemastine, clemizole, cyroheptadine, diphenylpyraline, doxylamine, flunarizine, mequitazine, meclozine, mepyramine, pheniramine, terfenadine, triprolidine, trimeprazine, ebastine, cinnarizine; Anti-migraines such as ergotamine, dihydroergotamine, methysergide, sumatriptan, naritriptan, almotriptan, zolmitriptan, rizatriptan, eletriptan, flumedroxone, pizotifen; Anti-tussives and dextromethorphan mucolytics such as pholcodeine, acetylcysteine, noscapine; Antineoplastics and azathiprine Immunosupressants such as methyluracil, fluorouracil, vincristine, vinblastine, melphalan, cyclophosphamide, aminoglutethimide, mercaptopurine, tamoxifen, chlorambucil, daunorubicin, mechlorethamine, doxorubicin; Anti-malarials such as quinine, chloroquine, pyrimethamine, amodiaquine, piperaquine, proguanil, chloroproguanil, mefloquine, primaquine, halofantrine; Anxiolytics, and Sedatives such as bromazepam; Hypnotics, and Antipsycotics such as nitrazepam, diazepam, oxazepam; Benzodiazepines such as clonazepam, chlorazepate, lorazepam, midazolam, triazolam, flunitrazepam; Butyrophenones such as droperidol, haloperidol; Barbiturates such as allobarbitone, aprobarbitone, phenobarbitone, amylobarbitone, barbitone, butobarbitone, zopiclone, hydroxyzine, buspirone, tandospirone, Bronchodilators such as theophylline; Cardiovascular Drugs including β-Blockers such as acebutatol, alprenolol, atenolol, labetalol, metopralol, nadolol, timolol, propanolol, pindolol, tolamolol, sotalol, oxprenolol, bunitrolol, carazolol, indenolol; Cardiovascular Drugs including Anti-arrythmics/cardiotonics such as disopyramide, cardiotonics, mexilitine, tocainide, aprindine, procainamide, quinidine, dobutamine; Cardiovascular Drugs including Ca channel blockers (all classes) including verapamil, diltiazem, amlodipine, felodipine, nicardipine, gallopamil, prenylamine; Cardiovascular Drugs including Antihypertensives/Vasodilators including diazoxide, guanethidine, clonidine, hydralizine, dihydralizine, minoxidil, prazosin, phenoxybenzamine, reserpine, phentolamine, perhexiline, indapamide, debrisoquine, bamethan, bethanidine, dobutamine, indoramin; Cardiovascular Drugs including Ace inhibitors captopril, enalapril, lisinopril, ramipril, imidapril; CNS stimulants/anorectics including methylphenidate, fenfluramine, amphetamine, methamphetamine, bemegride, caffeine, dexamphetamine, chlorphentamine, fencamfamine, prolintane; Diuretics such as furosemide, acetazolamide, amiloride, triampterene, bendrofluazide, chlorothiazide, chlorthalidone, cyclothiazide, hydroflumethiazide, hydrochlorothiazide, hydroflumethiazide; Gatrointestinal Agents including Motility enhancers, modulators and anti-emetics such as domperidone metoclopramide; cisapride, prochlorperazine, pirenzipine, cinitapride, cyclizine, chlorpromazine, prochloperazine, promethazine; Gatrointestinal Agents including Acid secretion modulators such as cimetidine, ranitidine, famotidine, omeprazole, nizatidine; Gatrointestinal Agents including Anti-diarrhealsincluding loperamide, diphenoxylate; Gatrointestinal Agents including emetics such as apomorphine; Muscle relaxants such as chlorzoxazon, rocuronium, suxamethonium, vecuronium, atracurium, fazadinium, doxacurium, mivacurium, pancuronium, tubocurarine, pipecurium, decamethonium, tizanidine, piridinol, succinylcholine, acetylcholine; Cholinergic Agents such as benzpyrinium, edrophonium, physostigmine, neostigmine, pyridostygmine; β-adrenergic agonists such as adrenaline ephedrine, pseudo-ephedrine, amidephrine, oxymetazoline, xylometazoline, terbutaline, salbutamol, salmeterol, phenylpropanolamine, cyclopentamine, phenylephrine, isoproterenol, fenoterol, xamoterol; Other CNS active agents such as dopamine, levodopa; Endocrine agents such as bromocriptine, propylthiouracil; Local anesthetics such as lidocaine (lignocaine), procaine, amethocaine, bupivacaine, butacaine, oxybuprocaine, mepivacaine, cocaine, prilocaine, amylocaine, chloroprocaine, cinchocaine, etidocaine, propoxycaine, tropacocaine, ropivacaine; Miscellaneous Mydriatics such as cyclopentolate, methazolamide, dorzolamide, acetazolamide, dynorphins, enkephalins, oxytocin and vasopressin. Additional basic therapeutic agents include naltrexone, varenicline, bacitracin, linezolid, daptomycin, granisetron, ondansetron, aripiprazole, risperidone, olanzapine, clozapine, thorazine, ipratropium, and bethanecol.

Preferably, the basic therapeutic agent is a local anesthetic such as, but not limited to: lidocaine (lignocaine), procaine, amethocaine, bupivacaine, butacaine, oxybuprocaine, mepivacaine, cocaine, prilocaine, amylocaine, chloroprocaine, cinchocaine, etidocaine, propoxycaine, tropacocaine, and ropivacaine. Preferably the basic therapeutic agent is lidocaine, bupivacaine or ropivacaine. Preferred acid addition salts of the invention include the the perfluoro-n-butane-1-sulfonate, perfluoro-n-pentane-1-sulfonate and perfluoro-n-hexane-1-sulfonate salts of lidocaine, bupivacaine and ropivacaine.

Suitable acidic drugs include, but are not limited to, meropenem, imipenem, ertapenem, prostacyclin, phenytoin, warfarin, tolbutamide, theophyllline, sulfapyridine, sulfadizine, salicyclic acid, propylthiouracil, phenobarbital, pentobarbital, peniciiamine, methyldopa, methotrexate, levodopa, ibuprofen, furosemide, ethacrynic acid, cephalexin, ciprofloxacin, chlorothiazide, aspirin, ampicillin, acetazolamide, and amoxicillin.

In certain embodiments, the basic drug and the acid do not form a true salt, i.e., an ionic compound. For example, a weak acid and a weak base can, under certain conditions, for a mixture, alloy or eutectic. Without being bound by theory, it is believed that in certain combinations of weakly basic drugs and weak acids, a mixture of the two results in which proton transfer from the acid to the base is incomplete. Such combinations can provide extended duration of release of the drug similar to salts when compositions are properly selected.

The compositions in accordance with the present invention provide, among other advantages, sustained or extended therapeutic levels of the drug following administration. Sustained release may be due to several factors including, but not limited to, the decreased solubility of the salt relative to the parent drug.

### V. Polymer Film Formulations

The compositions of the invention comprise particles of a poorly water-soluble drug embedded in a polymeric film. The compositions can be used to deliver the drug particles to a tissue or anatomical site of a subject in need of treatment with the drug.

The polymeric film preferably dissolves on contact with living tissues. In certain embodiments, the polymeric film melts at physiological temperature and then dissolves.

Preferably the polymeric film is bioresorbable. Preferably the polymeric film leaves substantially no residue at the anatomical site to which it is applied. More preferably, the polymeric film is bioresorbed and leaves substantially no residue after hours to one day of application. Preferably, the polymeric film does not substantially affect the uptake of the drug, i.e., the polymer film does not provide a matrix which substantially slows the release of the drug.

Suitable polymers for fabrication of the polymeric films of the invention include polyethylene glycol (PEG) of various molecular weights up to about 20,000, which would be expected to quickly dissolve under physiological conditions. Lower molecular weight PEG can also be used, including PEG with a molecular weight of 1000, which has a melting point of 34 to 36° C. Suitable polymers also include, but are not limited to, other water soluble polymers, such as homopolymers and copolymers, with molecular weights below 20,000, for example cellulose ethers, such as hydroxyethyl cellulose and hydroxypropyl cellulose; polyvinyl pyrrolidone; PEGylated polymers; polyvinyl alcohol; polyacrylamide; N-(2-hydroxypropyl)methacrylamide; divinyl ether-maleic anhydride; polyoxazoline; polyphosphates, polyphosphazenes; xanthan gum; pectins; chitosan derivatives, including N-acetyl chitosan; dextrans; carrageenans; guar gum; hyaluronic acid; albumin; starch and starch derivatives. The polymeric film can be composed of a single polymer or a combination of two or more polymers. In certain embodiments, the polymeric film is composed of a polymer blend.

In certain embodiments, the polymeric film is formed of multiple molecular weights of same polymer selected to provide desired chemical and/or physical properties. In certain embodiments, the polymeric film includes the polymer or polymers and a low molecular weight material for wetting of the drug particles which is combined with the polymer or polymers to enhance the mechanical properties of the film. For example, in certain embodiments the polymeric film includes PEG200 as a wetting agent, combined with PEG having a molecular weight of about 1,000 to 20,000. In certain embodiments, the particles are pre-treated with the wetting agent, such as PEG200, prior to embedding the particles in the polymeric film.

The polymeric film serves as a vehicle for administration of the drug to an anatomic site, for example, a biological surface, such as a wound bed, preferably resulting in a substantially uniform distribution of the drug particles to the biological surface. Preferably, the polymeric film melts, dissolves and/or degrades rapidly following administration to a subject and does not affect the uptake of the drug by the subject.

The polymer film compositions of the invention enable delivery of a predetermined amount of a drug salt to a tissue or other anatomical site of the body of a subject in need of treatment with the drug. Following application, the polymeric film preferably dissolves and/or melts at the application site, and the polymeric material serves to wet the hydrophobic salt particles and inhibit particle aggregation.

In certain embodiments, the polymeric film composition of the invention is provided in the form of a sheet, and the drug or drug salt particles are substantially uniformly distributed through the sheet. In certain embodiments, the sheet contains the maximum allowable dose of the drug. The sheet can be cut into smaller pieces to fit the anatomic site or to control the dose administered. Under conditions of use in which the anatomic site is too small to permit the dosing of the desired amount of drug with a single sheet or portion thereof, the polymeric film composition can be administered as two or more sheet layers. For example, when the polymeric film composition comprises a local anesthetic, as described below, the composition can be used to treat pain at a small but particularly painful location, such as surgical sites for bunionectomies and treatment of bone spurs and toe deformities, by placing two or more appropriately sized sheets in layers at the anatomic site.

The polymeric film compositions of the invention can optionally include a detectable compound, such as a dye, for example a visible or ultraviolet dye, or a fluorescent or luminescent compound to enhance visibility of the polymeric film before and after placement at the desired anatomic site. Preferred dyes are highly colored and visible in the presence of blood Preferred fluorescent compounds fluoresce under ultra-violet light. See, for example, Richard P. Haugland; Molecular Probes—Handbook of Fluorescent Probes and Research Chemicals; 5th Edition 1992-94; Molecular Probes, Inc.

The polymeric film compositions of the invention can optionally include markings, such as lines, which denote particular doses of the drug. Such markings allow the healthcare professional to cut the polymeric film composition to provide a piece of the composition which provides the desired dose of the drug.

The polymeric film is preferably composed of a polymeric material or combination of polymeric materials and is of a thickness which allows handling of the polymeric film composition by a health care provider. For example, the polymeric sheet preferably has suitable mechanical properties to be manipulated by hand and/or with forceps.

The polymeric film can be of any suitable thickness, but is preferably about 3 mm or less. In certain embodiments, the polymeric film has a thickness which is about 1.5 mm or less. More preferably, the polymeric film has a thickness of about 1 mm or less, such as about 0.1 to 0.9 mm, about 0.25 to 0.8 mm, or about 0.25 to 0.6 mm.

The loading of the drug in the polymeric film can vary. Preferably, the drug is substantially uniformly dispersed throughout the film such that for given amount of film by surface area, the amount of drug will be substantially constant throughout the film. For example, the polymeric film composition of the invention can be from about 0.1 to about 75 weight percent drug or drug salt, preferably from about 1 to about 50 weight percent drug or drug salt. In certain embodiments, the amount of drug or drug salt in the polymeric film can be about 0.1 mg per cm³ of polymeric film, about 1 mg or greater per cm³ of the polymeric film, about 4 mg or greater per cm³ of the polymeric film or about 10 mg or greater per cm³ of the polymeric film. In certain embodiments, the amount of drug or drug salt is from about 0.1 mg to about 25 mg per cm³ of the polymeric film.

The salts as described herein are preferably present in the polymeric film compositions of the invention in the form of particles. The particles can further comprise one or more pharmaceutically acceptable excipients or additives, such as surfactants, polymers and salts. Preferably, the particles do not include a matrix, such as polymer matrix, which prolongs release of the drug.

The size distribution of a particle composition of a salt of the invention will generally have at least about 50 weight % within 75%, more usually within 50%, and desirably within 25% of the median size. The median size will generally range from about 1 to about 2000 m, more usually from about 5 to 1500 m, desirably from about 5 m to 1200 m. Individual compositions of interest have median sizes of about 1 to 25 m; 5 to 100 m; 100 to 200 m, 300 to 500 m 500 to 750 m, 600 to 700 m and 750 to 1200 m. In one embodiment, the median size of the particles is about 625 to 675 m, or about 650 m.

Depending upon the manner in which the particles are made, they can comprise less than about 2, more usually less than about 1, weight % of the solvent used in their preparation, and preferably undetectable amounts.

In certain embodiments, the polymeric film composition comprises particles of an anesthetic, for example, a local anesthetic, or a salt thereof. Such compositions are useful, for example, for the treatment of pain due to an injury, particularly a wound. Preferably, the particles comprise as their major ingredient the anesthetic or a salt thereof with a hydrophobic acid. In preferred embodiments, the local anesthetic is a “caine” anesthetic. Examples of such anesthetics of the caine family include lidocaine (lignocaine), procaine, bupivacaine, ropivacaine, butacaine, oxybuprocaine, mepivacaine, prilocaine, amylocaine, chloroprocaine, etidocaine, propoxycaine and tropacocaine. Caines of particular interest are lidocaine, bupivacaine and ropivacaine.

In certain embodiments, the particles consist of one or more caine salts as described herein, or consist essentially of one or more such caine salts. In certain embodiments, the particles can have a 1:1 equivalent ratio of the anesthetic to the acid or one of the components may be in excess, usually not more than about 5-fold excess, generally up to about 0.5, or up to about a 0.2, equivalent excess of either of the components of the salt may be present. In certain embodiments, the particles include excess acid. By having excess acid, the release rate of the caine from the salt may be diminished by virtue of the common ion effect, where the dissolution of the excess acid will act to slow or retard the dissolution rate of the caine salt compound in the particles. In other embodiments, the particles include two caine salts as described herein, wherein preferably the two caine salts have different aqueous solubilities.

The particles can further comprise two or more caine salts of the invention, differing in either or both of the caine agent and the acid. For example, the particles can comprise two or more caine salts as described herein wherein the counter ions are different and which differ in hydrophobicity. By using different acid addition salts, the rate of release of the anesthetic can be modulated, with acids with smaller R groups usually providing for more rapid release. The composition may be a mixture of different sized particles, usually comprising not more than two different distributions, where each of the different distributions has at least about 75% of the weight of the particles within 50%, more usually within 25%, of the median weight. The median weights of the two differently sized compositions will usually differ by at least about 25%, more usually at least about 50% and there may be a two-fold difference or greater. In this way both composition and particle size can be varied to provide the optimum release profile for the particular application for the subject compositions.

In one embodiment, the polymer film composition comprises particles of a caine salt of Formula (VIII) and a soluble salt of the caine or a different caine. The soluble caine salt can be in a solid form, for example, in the form of particles, or in solutionThe soluble caine salt is preferably the hydrochloride, hydrobromide, acetic acid or nitric acid salt, preferably the hydrochloride salt. For example, the composition can comprise a salt of lidocaine, bupivacaine or ropivacaine of Formula VIII and a soluble salt of one of these caines, such as lidocaine hydrochloride, bupivacaine hydrochloride or ropivacaine hydrochloride. Preferably, the same caine is present in both salts. Such compositions provide both a rapid onset of action due to the soluble salt and sustained action due to the caine salt as described herein.

The polymeric film compositions of the invention comprising a caine anesthetic or salt thereof are particularly useful for the treatment of pain. In certain embodiments, the pain is due to a wound, such as a wound due to trauma or surgery. In one embodiment, the salts are useful for the topical treatment of a wound, for example, a surface wound resulting from trauma or surgery. In treating the wound, the polymeric film composition is administered directly onto the wound bed and onto the tissue for an open wound, for example.

In addition to the drug particles and the polymeric film, the compositions of the invention can include suitable pharmaceutically acceptable excipients to enhance the handling properties of the film and/or the activity or absorption the drug.

Suitable viscosity adjusting agents for use in the polymeric film compositions of the present invention include, but are not limited to protective colloids or non-ionic gums such as hydroxyethylcellulose, xanthan gum, magnesium aluminum silicate, silica, microcrystalline wax, beeswax, paraffin, and cetyl palmitate, or combinations or mixtures thereof.

Suitable surfactants for use in the polymeric compositions of the present invention include, but are not limited to, nonionic surfactants. For example, dimethicone copolyol, polyethylene glycols, including higher PEGs, such as PEG200, polysorbate 20, polysorbate 40, polysorbate 60, polysorbate 80, lauramide DEA, cocamide DEA, and cocamide MEA, are contemplated for use with the formulations of the present invention. In addition, combinations or mixtures of these surfactants can be used in the formulations of the present invention.

Suitable preservatives for use in the polymeric film compositions of the present invention include, but are not limited to antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. In addition, combinations or mixtures of these preservatives can be used in the formulations of the present invention.

Suitable moisturizers for use in the polymeric film compositions of the present invention include, but are not limited to lactic acid and other hydroxy acids and their salts, glycerin, propylene glycol, and butylene glycol. Suitable emollients include lanolin alcohol, lanolin, lanolin derivatives, lipids, phospholipids, cholesterol, petrolatum, isostearyl neopentanoate and mineral oils. In addition, combinations or mixtures of these moisturizers and emollients can be used in the formulations of the present invention.

The amount of the anesthetic salt applied to the wound area will be a therapeutically effective amount to minimize pain to a level that the patient can tolerate and preferably substantially eliminate any sense of pain. The amount of pain will usually vary with time, so that the amount of anesthetic that will be required can be diminished over time. Therefore, the profile of anesthetic release from the salt can be a diminishing amount of anesthetic being released over time. Conveniently, there may be an initial large release, less than about 30%, usually less than about 25%, of the total amount of anesthetic followed by a decreasing release over time at a lower amount at a therapeutic level. The large initial release coincides with the high levels of pain in the early post-operative period. After the initial release, generally not more than 60 weight %, more usually not more than about 50 weight %, will be released in 24 hours, where the pain alleviation is to occur over generally greater than two days, with diminishing percentages as the time for relief is extended.

Once the particles have been prepared, irrespective of the method employed in their preparation, the particles are sized and fractioned typically by sieving operations, although other methods may be employed. To control particle distribution and particle size a typical sieving operation would employ at least 2 sieves of the appropriate size. The larger sieve size would allow for the rejection of particles larger than the specified maximum while the lower sieve size would serve to retain the particles of the specified size. The selection of the sieves determines the particle size distribution. Using this approach one can also prepare multimodal distributions to obtain different release profiles of drug. Nominal particle size and particle size distribution is determined by an instrument such as a Coulter LS13 on suspensions of the microparticles.

Drug dissolution kinetics is evaluated using an LC method employing an infinite sink concept. A known amount of microparticles are suspended in a defined volume of a suitable test medium, for example a phosphate buffer solution containing 1% Tween 80, meant to simulate in vivo release kinetics. The suspension of microparticles is kept at a constant temperature, typically 37° C., for a period of time, for example, about 12 hours, with constant agitation. The particles are removed from the solution by filtration and re-suspended in another fresh amount of the test media. The original solution is assayed for the amount of drug product in solution by an appropriate quantitative method, typically an LC method employing UV detection or MS.

If fluorescent or colored microparticles are desired the procedure for making the microparticle is followed, however, for a fluorescent product a compound such as fluorescein is added to the mixture before the precipitation or preparation of the microparticle is attempted. If a colored product is required a food safe dye such as FD&C Blue No 1 or Blue No 2 is used.

The acid addition salts of the invention can be prepared by methods known in the art. For example, an acid addition salt of a basic drug in accordance with the invention may be prepared by any conventional means, including precipitation of the salt from solution, spray drying a solution of the salt, reaction of the drug and acid in solution and removal of solvent, or fusion of the free base of the drug with the acid. In one embodiment the free base of the drug compound is combined with the acid in a suitable solvent, such as water or a polar organic solvent. Alternatively, a salt of the drug, such as the hydrochloride salt, is reacted with a salt of the acid, for example, the sodium salt, in water or a polar organic solvent. In either case, the desired salt can either spontaneously precipitate upon formation or be induced to precipitate by adding a suitable cosolvent and/or concentrating the solution. In certain embodiments, the free base of the drug is combined with the acid in the absence of solvent, resulting in the formation of the desired salt.

The polymeric film compositions of the invention can be prepared using any method which embeds the drug or drug salt particles in the film. For example, in certain embodiments, the polymer has a melting point between room temperature and physiological temperature and is warmed to its melting point or higher. The drug particles and, optionally, excipients, are added to the melted polymer. The resulting mixture is preferably stirred to distribute the particles and optional excipients substantially uniformly throughout the melted polymer. The mixture is then added to a mold and allowed to cool to form a film of the desired thickness. Alternatively, the liquid mixture can be poured onto a substrate, such as a polymer sheet, for example, a polyethylene sheet, and spread on the substrate to form a film of a desired thickness.

### VI. Formulations of Drug Particles and Wetting Agents

In a fifth embodiment, the present invention provides compositions comprising particles of a drug and at least one wetting agent. The compositions can be used to deliver the drug particles to a subject in need of treatment with the drug.

The drug can be a free acid, a free base or in the form of a salt as described herein.

The size distribution of the hydrophobic drug particles of the compositions of the invention will generally have at least about 50 weight % within 75%, more usually within 50%, and desirably within 25% of the median size. The median size will generally range from about 1 to about 2000 m, more usually from about 5 to 1500 m, desirably from about 5 m to 1200 μm. Individual compositions of interest have median sizes of about 1 to 25 μm; 5 to 100 μm; 100 to 200 μm, 300 to 500 μm, 500 to 750 μm, 600 to 700 μm and 750 to 1200 μm. In one embodiment, the median size of the particles is about 625 to 675 μm, or about 650 μm.

Depending upon the manner in which the particles are made, they can comprise less than about 2, more usually less than about 1, weight % of the solvent used in their preparation, and preferably undetectable amounts.

The wetting agent is an excipient which prevents or inhibits aggregation of the particles. Suitable wetting agents include nonionic, amphoteric and ionic wetting agents, such as polyhydroxy compounds, including saccharides and sugar alcohols; polyethers, including polyethylene glycols (PEGs) and polypropylene glycols; and non-ionic surfactants, such as poloxamers. Examples of wetting agents include polysorbate, sorbitan esters, sorbitol, propylene glycol, and poloxamers. Preferred wetting agents include polyethylene glycols having a molecular weight from about 100 amu to about 10,000 amu or from about 100 amu to about 1,000 amu. The PEG can be linear or branched. A particularly preferred polyethylene glycol is PEG200. In certain embodiments, the wetting agent is selected to be soluble in the liquid vehicle. In certain embodiments, the wetting agent is a solid under conditions of formulation and use. In cetain embodiments, the wetting agent is a solid under conditions of formulation, but melts at physiological temperature. The amount of wetting agent in the composition is preferably sufficient to substantially inhibit aggregation of the particles.

In certain embodiments, the hydrophobic drug particles are suspended in a liquid wetting agent. In another embodiment, the particles are suspended in a vehicle, such as a liquid, paste, lotion or gel. Suitable vehicles include, but are not limited to water, propylene glycol, polyethylene glycols, polypropylene glycols and mixtures thereof. The vehicle can also be an aqueous solution, such as an aqueous buffer, normal saline or buffered saline. Preferably, not more than about 10 weight %, and usually not more than 5 weight %, of the hydrophobic drug will be soluble in the vehicle; preferably the hydrophobic drug is substantially insoluble in the medium.

In preferred embodiments, the hydrophobic drug is substantially insoluble in the liquid vehicle and the wetting agent is soluble in the liquid vehicle. Preferably, the hydrophobic drug particles are suspended in a solution of the wetting agent in the vehicle.

In certain embodiments, the hydrophobic drug particles are coated with the wetting agent or agents before they are suspended in the vehicle.

In certain embodiments, the hydrophobic drug particles are mixed with a solid wetting agent. Preferably, the solid wetting agent is in the form of particles. More preferably, the size of the wetting agent particles is substantially the same as the size of the hydrophobic drug particles. The solid wetting agent can be any wetting agent which is a solid at room temperature, i.e., at about 25° C. or at physiological temperature, i.e. about 37° C. In one embodiment, the wetting agent is a solid under conditions of formulation, storage and administration, but melts following administration. In another embodiment, the wetting agent remains a solid after administration. In certain embodiments, the solid wetting agent is a solid polyethylene glycol, such as a PEG having a molecular weight of about 1000 amu or greater, preferably from about 1000 amu to about 10,000 amu, and more preferably about 2500 amu to about 7500 amu. In one embodiment, the PEG can have a molecular weight of about 3000 amu to about 3500 amu, or about 3350 amu. In another embodiment, the PEG has a molecular weight of about 5000 to 7000 amu, or about 6000 amu.

The particles of the hydrophobic drug and the particles of the wetting agent can be mixed in any suitable ratio. In certain embodiments, the weight ratio of drug particles to wetting agent particles is from 1/3 to 9.5/1, or about 1/2 to about 9/1. In another embodiment, the ratio is from about 1/1 to about 9/1.

In certain embodiments, the composition of the invention comprises particles of an anesthetic, for example, a local anesthetic, or a salt thereof. Such compositions are useful, for example, for the treatment of pain due to an injury, particularly a wound. Preferably, the particles comprise as their major ingredient the anesthetic or a salt thereof with a hydrophobic acid. In preferred embodiments, the local anesthetic is a “caine” anesthetic. Local anesthetics of the “caine” family are weak monobases. (by “caine” is intended anesthetics that end in the suffix “caine”, which in certain embodiments include an amino acid amide or ester). One of the classes of caine anesthetics are amine bases and also include an aromatic ring, for example, a meta-xylyl group, and an amide or ester functionality. The aromatic group with the other entities results in hydrophobicity, so that the members of the class are frequently employed as their hydrochloride salts to allow for water solubility. Examples of such anesthetics of the caine family include lidocaine (lignocaine), procaine, bupivacaine, ropivacaine, butacaine, oxybuprocaine, mepivacaine, prilocaine, amylocaine, chloroprocaine, etidocaine, propoxycaine and tropacocaine. Caines of particular interest are lidocaine, bupivacaine and ropivacaine.

In certain embodiments, the particles consist of one or more caine salts as described herein, or consist essentially of one or more such caine salts. In certain embodiments, the particles can have a 1:1 equivalent ratio of the anesthetic to the acid or one of the components may be in excess, usually not more than about 5-fold excess, generally up to about 0.5, or up to about a 0.2, equivalent excess of either of the components of the salt may be present. In certain embodiments, the particles include excess acid. By having excess acid, the release rate of the caine from the salt may be diminished by virtue of the common ion effect, where the dissolution of the excess acid will act to slow or retard the dissolution rate of the caine salt compound in the particles. In other embodiments, the particles include two caine salts as described herein, wherein preferably the two caine salts have different aqueous solubilities.

The particles can further comprise two or more drug salts, differing in the counterion. For example, the particles can comprise two or more caine salts as described herein in which the counterions are different and which differ in hydrophobicity. By using different acid addition salts, the rate of release of the anesthetic can be modulated, with acids with smaller R groups usually providing for more rapid release. The composition can be a mixture of different sized particles, usually comprising not more than two different distributions, where each of the different distributions has at least about 75% of the weight of the particles within 50%, more usually within 25%, of the median weight. The median weights of the two differently sized compositions will usually differ by at least about 25%, more usually at least about 50% and there may be a two-fold difference or greater. In this way both composition and particle size can be varied to provide the optimum release profile for the particular application for the subject compositions.

In one embodiment, the composition comprises particles of a caine salt as described herein and a soluble salt of the caine or a different caine. The soluble caine salt can be in a solid form, for example, in the form of particles, or in solution. The soluble caine salt is preferably the hydrochloride, hydrobromide, acetic acid or nitric acid salt, preferably the hydrochloride salt. For example, the composition can comprise a salt of lidocaine, bupivacaine or ropivacaine as described herein and a soluble salt of one of these caines, such as lidocaine hydrochloride, bupivacaine hydrochloride or ropivacaine hydrochloride. Preferably, the same caine is present in both salts. Such compositions provide both a rapid onset of action due to the soluble salt and sustained action due to the caine salt of the hydrophobic acid.

Most common propellants are mixtures of volatile hydrocarbons, typically propane, n-butane and isobutane, or hydrofluoroalkanes (HFA): either HFA 134a (1,1,1,2,-tetrafluoroethane) or HFA 227 (1,1,1,2,3,3,3-heptafluoropropane) or combinations of the two or compressed gases such as nitrogen, carbon dioxide, air and the like. One may also use a simple air brush means of dispensing the particles where there is literally no solvent but air is drawn and used to dispense the particles.

The compositions of the invention can additionally include one or more additives or excipients. Such additives include protein-based materials such as collagen and gelatin; silicone-based materials; stabilizing and suspending agents; emulsifying agents; and other vehicle components that are suitable for administration to the skin, as well as mixtures of these components and those otherwise known in the art. The vehicle can further include components adapted to improve the stability or effectiveness of the applied formulation, such as preservatives, antioxidants, and skin penetration enhancers. Examples of such components are described in the following reference works hereby incorporated by reference: Martindale—The Extra Pharmacopoeia (Pharmaceutical Press, London 1993) and Martin (ed.), Remington's Pharmaceutical Sciences.

The topical formulations of the caine salts of the invention can be prepared in a variety of physical forms. For example, solid particles, pastes, creams, lotions, gels, and liquids are all contemplated by the present invention. A difference between these forms is their physical appearance and viscosity, which can be governed by the presence and amount of emulsifiers and viscosity adjusters present in the formulation. Particular topical formulations can often be prepared in a variety of these forms. Solids are generally firm and will usually be in particulate form; solids optionally can contain liquids, emulsifiers, moisturizers, emollients, fragrances, dyes/colorants, preservatives and other active ingredients that increase or enhance the efficacy of the final product. Creams and lotions are often similar to one another, differing mainly in their viscosity; both lotions and creams may be opaque, translucent or clear and often contain emulsifiers, solvents, and viscosity adjusting agents, as well as moisturizers, emollients, fragrances, dyes/colorants, preservatives and other active ingredients that increase or enhance the efficacy of the final product. Gels can be prepared with a range of viscosities, from thick or high viscosity to thin or low viscosity. These formulations, like those of lotions and creams may also contain liquids, emulsifiers, moisturizers, emollients, fragrances, dyes/colorants, preservatives and other ingredients that increase or enhance the efficacy of the final product. Liquids are thinner than creams, lotions, or gels and often do not contain emulsifiers.

Suitable emulsifiers for use in the caine addition salt formulations of the present invention include, but are not limited to ionic emulsifiers, behentirmonium methosulfate, cetearyl alcohol, non-ionic emulsifiers like polyoxyethylene oleyl ether, PEG-40 sterate, ceteareth-12, ceteareth-20, ceteareth-30, ceteareth alcohol, PEG-100 stearate, glyceryl stearate, or combinations or mixtures thereof.

Suitable viscosity adjusting agents for use in the caine salt formulations of the present invention include, but are not limited to protective colloids or non-ionic gums such as hydroxyethylcellulose, xanthan gum, magnesium aluminum silicate, silica, microcrystalline wax, beeswax, paraffin, and cetyl palmitate, or combinations or mixtures thereof.

Suitable liquids for use in the caine salt formulations of the present invention will be selected to be non-irritating and include, but are not limited to water, propylene glycol, polyethylene glycols, polypropylene glycols and mixtures thereof. Not more than about 10 weight %, usually not more than 5 weight %, of the anesthetic salt will be soluble in the medium; preferably the anesthetic salt will be insoluble in the medium.

Suitable surfactants for use in the caine salt formulations of the present invention include, but are not limited to nonionic surfactants. For example, dimethicone copolyol, polyethylene glycols, including higher PEGs, such as PEG200, polysorbate 20, polysorbate 40, polysorbate 60, polysorbate 80, lauramide DEA, cocamide DEA, and cocamide MEA, are contemplated for use with the formulations of the present invention. In addition, combinations or mixtures of these surfactants can be used in the formulations of the present invention.

Suitable preservatives for use in the caine salt formulations of the present invention include, but are not limited to antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. In addition, combinations or mixtures of these preservatives can be used in the formulations of the present invention.

Suitable moisturizers for use in the caine salt formulations of the present invention include, but are not limited to lactic acid and other hydroxy acids and their salts, glycerin, propylene glycol, and butylene glycol. Suitable emollients include lanolin alcohol, lanolin, lanolin derivatives, lipids, phospholipids, cholesterol, petrolatum, isostearyl neopentanoate and mineral oils. In addition, combinations or mixtures of these moisturizers and emollients can be used in the formulations of the present invention.

Other suitable additional ingredients that may be included in the caine salt formulation of the present invention include, but are not limited to, abrasives, absorbents, anticaking agents, anti-foaming agents, anti-static agents, astringents, binders/excipients, buffering agents, chelating agents, film forming agents, conditioning agents, opacifying agents, pH adjusters and protectants. Examples of each of these ingredients in topical product formulations, can be found in publications by The Cosmetic, Toiletry, and Fragrance Association (CTFA). See, e.g., CTFA Cosmetic Ingredient Handbook, 2^{nd} edition, eds. John A. Wenninger and G. N. McEwen, Jr. (CTFA, 1992).

In many instances it may be desirable that the health care professional administering the particle formulation is able to insure uniform coverage or otherwise be able to see what areas have been covered and how extensively the particle formulation has been distributed.

Therefore, one may include a detectable composition with the particles so that they can be visualized. This may include colored compounds or dyes, fluorescent compounds and even luminescent compounds. The dyes should be highly colored and visible in the presence of blood, while the fluorescent compounds should fluoresce under ultra-violet light. See, for example, Richard P. Haugland; Molecular Probes-Handbook of Fluorescent Probes and Research Chemicals; 5th Edition 1992-94; Molecular Probes, Inc.

The particles will typically be at least about 1 weight %, usually at least 2 weight %, and up to 100 weight % of the non-volatile portion of the composition. Where the particles are dispersed in a vehicle, the weight % of the particles will generally be in the range of about 1-75 weight %, more usually about 1-50 weight %. The minor ingredients except for the medium will generally range from about 0.01 weight % to about 10 weight %, the amount generally being conventional for the purpose of the ingredient. Where the particles are sprayed as an aerosol, generally the particles will be present in the range of about 1 to 99 weight % of the composition.

Depending upon the need and the nature of the composition, the composition may be sprayed, wiped, smeared, painted, transferred from a template onto or proximal to the wound or may be made into a patch where the composition will be separate from or part of the adhesive. Alternatively, topically the composition may be applied to the wound and a dressing or other protective layer added to prevent contamination and abrasion. In some situations, the composition may be injected, particularly where a minimally invasive surgical technique is employed and the rate of transdermal transport is insufficient to provide the pain relief required. Not more than one application should be required per 6 hours, usually per half-day, and times between applications may vary from 6 hours to 7 days, usually 12 hours to 4 days, where frequently, by 7 days, further treatment will not be required. During this time a therapeutically effective amount of the caine will be released from the particles.

The amount of the anesthetic salt applied to the wound area will be a therapeutically effective amount to minimize pain to a level that the patient can tolerate and preferably substantially eliminate any sense of pain. The amount of pain will usually vary with time, so that the amount of anesthetic that will be required can be diminished over time. Therefore, the profile of anesthetic release from the salt can be a diminishing amount of anesthetic being released over time. Conveniently, there may be an initial large release, less than about 30%, usually less than about 25%, of the total amount of anesthetic followed by a decreasing release over time at a lower amount at a therapeutic level. The large initial release coincides with the high levels of pain in the early post-operative period. After the initial release, generally not more than 60 weight %, more usually not more than about 50 weight %, will be released in 24 hours, where the pain alleviation is to occur over generally greater than two days, with diminishing percentages as the time for relief is extended.

Once the particles have been prepared, irrespective of the method employed in their preparation, the particles are sized and fractioned typically by sieving operations, although other methods may be employed. To control particle distribution and particle size a typical sieving operation would employ at least 2 sieves of the appropriate size. The larger sieve size would allow for the rejection of particles larger than the specified maximum while the lower sieve size would serve to retain the particles of the specified size. The selection of the sieves determines the particle size distribution. Using this approach one can also prepare multimodal distributions to obtain different release profiles of drug. Nominal particle size and particle size distribution is determined by an instrument such as a Coulter LS13 on suspensions of the microparticles.

Drug dissolution kinetics is evaluated using an LC method employing an infinite sink concept. A known amount of microparticles are suspended in a defined volume of a suitable test medium, for example a phosphate buffer solution containing 1% Tween 80, meant to simulate in vivo release kinetics. The suspension of microparticles is kept at a constant temperature, typically 37° C., for a period of time, for example, about 12 hours, with constant agitation. The particles are removed from the solution by filtration and re-suspended in another fresh amount of the test media. The original solution is assayed for the amount of drug product in solution by an appropriate quantitative method, typically an LC method employing UV detection or MS.

If fluorescent or colored microparticles are desired the procedure for making the microparticle is followed, however, for a fluorescent product a compound such as fluorescein is added to the mixture before the precipitation or preparation of the microparticle is attempted. If a colored product is required a food safe dye such as FD&C Blue No 1 or Blue No 2 is used.

Drug product of the appropriate size is combined with other agents that may be appropriate to provide free flowing stable microparticles and added to an appropriate aerosol container. The aerosol container is subsequently pressurized with a high purity propellant and sealed under pressure with the appropriate spray nozzle to provide the spray pattern desired and in some cases to provide a metered dose of the drug. Alternatively, the drug product can be suspended into a PBS solution or other suitable vehicle just prior to application to the wound. The product is distributed over the wound by spraying using a variety of possible propulsion systems e.g., an air brush type of system, pump sprayer system, etc., whereby drug product suspended in the PBS is aspirated through a tube using the Venturi concept with a propellant container.

The acid addition salts of the invention can be prepared by methods known in the art. For example, an acid addition salt of a basic drug in accordance with the invention may be prepared by any conventional means, including precipitation of the salt from solution, spray drying a solution of the salt, reaction of the drug and acid in solution and removal of solvent, or fusion of the free base of the drug with the acid. In one embodiment the free base of the drug compound is combined with the acid in a suitable solvent, such as water or a polar organic solvent. Alternatively, a salt of the drug, such as the hydrochloride salt, is reacted with a salt of the acid, for example, the sodium salt, in water or a polar organic solvent. In either case, the desired salt can either spontaneously precipitate upon formation or be induced to precipitate by adding a suitable cosolvent and/or concentrating the solution. In certain embodiments, the free base of the drug is combined with the acid in the absence of solvent, resulting in the formation of the desired salt.

### VII. Polymeric Drug Delivery Device

In a sixth embodiment, the present invention provides a formulation comprising a drug or salt thereof incorporated into polymeric structures having at least one opening which allows the interior of the structure to communicate with the environment. In a preferred embodiment, aminoacid amides and ester, such as caine anesthetics, optionally in the form of acid addition salts (caine salts), are incorporated into rate controlling delivery tubes for the purposes of sustained release of the anesthetic aminoacid amide or ester, hereafter referred to as caine salts. These tubes can be applied to the tissue directly or incorporated into dressings, bandages, creams, ointments, gels and lotions to provide for the extended release of an anesthetic agent over many days. The rate of drug release is determined by the diameter of the tubes containing the anesthetic caine and the inherent solubility of the caine itself depending upon the salt form chosen. The duration of caine release is determined by the length of the tube.

A tube of a defined diameter is chosen for the release flux and duration for a specific indication. The rate of delivery of the drug from the tube is proportional to the surface area of face or faces of the open ended tube and the inherent solubility of the drug. In general, the rate of dissolution is dependent upon the surface area to volume ratio of any substance. A spherically shaped objected from which dissolution takes place from the entire surface will show a progressively decreasing rate of release as the sphere shrinks in size and the surface area is reduced. Similarly, a rod shaped solid drug salt particle will show a decrease in the rate of release characteristic of its geometric shape and the surface area to volume ratio. Limiting the dissolution to the surface of a three dimensional object will only allow dissolution in 2 dimensions. The release from such a surface only shape will therefore be constant with time. This is characterized as a zero order release and may be desirable for some drug delivery applications.

Other geometric shapes may also be employed to control the release kinetics of the anesthetic agent. Other shapes such as cubes, rectangles, cones, prisms, tetrahedrons, octahedron or any other shapes as may be readily derived may also be used in place of the aforementioned tube. Other shapes with open faces will provide other release kinetics as may be calculated by those skilled in the art providing a unique therapeutic release profile.

Although the discussion for the rate controlled delivery of a caine anesthetic has been for tubes, any geometric shape may be employed for use in this invention. As examples one may employ a sphere with a hole, a cone with the base face exposed, a cube or rectangle with a face exposed. These and many other geometric shapes may be employed and all will provide a unique drug delivery profile dependent on the shape of caine containing object, the surface area exposed and the solubility of the caine employed. The delivery from such objects is readily calculated by those skilled in the art and can provide unique delivery profiles that may be desirable for certain applications.

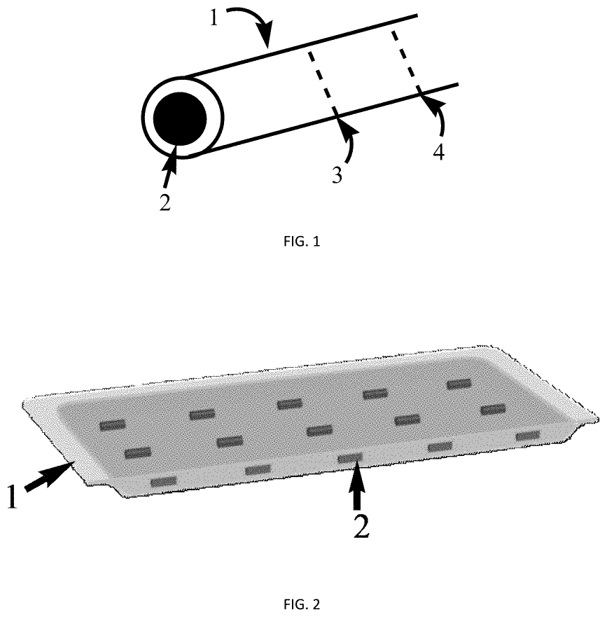

This invention employs a drug within an insoluble tube allowing for the exposure of the end faces of the tube to an aqueous environment allowing for the dissolution of the drug contained within. This type of configuration is represented in FIG. 1, which shows polymeric tube (1), an open end of the tube (2) which communicates with the drug reservoir and exemplary truncation siutes 3 and 4. In such a configuration dissolution of the drug will only take place on each cut end or face. As dissolution of the drug continues the drug will continue to erode down the tube continuously exposing new drug to the aqueous environment and providing a zero order release of the drug.

A larger diameter tube of drug will allow for a greater amount of drug delivered per unit time as the dissolution rate will be determined by the exposed surface area. The invention therefore allows for a wide range of drug delivery rates that depend upon the diameter of the tube used. Applications that require a small amount of drug to be delivered per unit of time will employ small diameter tubes. Applications requiring larger amounts of drug will use larger diameter tubes. This can be mathematically determined in advance knowing the drug dissolution rate per unit of exposed surface and by calculation knowing the desired drug concentration one may readily determine the amount of tubes of specified diameter to be used in the application.

The duration of release is controlled through the length of the tubes of drug employed. Longer tubes result in longer duration of release. Using both the tube diameter and the tube length allows one to design a drug release profile for any given amount of drug for any duration. The selection of tube diameter and tube length allows for the facile design of products that will last from hours to weeks and which can be readily calculated once one knows the dissolution rate of the drug in terms of mass released per unit time and unit area.

The use of an insoluble tube is not necessary if a relatively non-permeable coating is employed to provide a similar effect as a tube. The concept of a tube is used to describe a material which will allow little water or drug diffusion while retaining the drug in a reservoir. Many materials and designs can be envisioned as meeting these criteria. The tube may actually be a physical tube which is filled with a drug and is made of a thermoplastic materials such as polyethylene, polypropylene, nylon, polyester, urethane and generally of any material know to those skilled in the art that will maintain its structural properties while allowing for little diffusion of water into the tube or drug out of the tube. The tube is not a part of the delivery kinetics other than to act as a reservoir for remaining drug and allow the drug to dissolve from each exposed end surface of the tube.

The tube may also be made from a bioresorbable polymer meeting the aforementioned characteristics. A bioresorbable material would be one in which the tube material decomposes or degrades after the drug has eluted from the device. Such a material provides the benefit where it would be desirable to have no physically remaining tube after some period of time. One such example would be the use in a wound where the tubes may become incorporated into the wound with healing. Bioresorbable polymers such as polyesters, polyamides, polycarbonates and other materials known to those skilled in the art can be employed. The polymer may erode or absorb though either a bulk or surface degradation mechanism so long as it remains mostly intact for the duration of the drug delivery.

Additionally, the tube may be prepared from thermoset materials if a particular longevity of the drug tubes is desired or if manufacturing of the drug product using such thermosets provides a design advantage. Any thermoset providing the aforementioned tube characteristics would be suitable such as epoxies, polyesters, polyurethanes and other polymeric materials that would be known to those skilled in the art.

Additionally, the tube may be made from a bioresorbable inorganic material such as hydroxyapatite or combinations of an inorganic material and an organic polymer or inorganic polymer such as silicone to provide flexibility. The inorganic material may also be combined with bioresorbable organic polymers as described previously. Such a system may find use for bone surgery where the caine anesthetic would be part of the repair materials. Other materials known to those skilled in the art may also be employed in a similar manner.

The drug filled tubes used in the fabrication of a device may be prepared by a variety of techniques. Tubes may be filled using a molten form of the drug by injection filling or other means to introduce the molten drug into the tube. Once filled the drug filled tubes can be cut to length. Alternatively, drug may be coextruded with a suitable plastic allowing for the simultaneous formation of drug filled tubing. This tubing may be subsequently cut to the appropriate length either during the formation of the drug filled tube or after the tubing has been prepared. Alternatively, a molten form or a cooled tube wire form of the drug may be spray coated with an appropriate solution of a polymer meeting the described characteristics. This method allows for thin tube construction. Alternatively, a drug extrusion may be coated by dipping or otherwise passing the molten drug through an appropriate molten polymer or solution of a polymer.

The drug containing tubes are incorporated into a device or into a topical or surgical product and become activated when wet. As one example the drug tubes can be added to a topical dressing or bandage to provide continuous release of an anesthetic caine drug. This is shown by example in FIG. 2, where the drug tubes (2) are uniformly dispersed in the dressing material (1).

When the dressing is wetted, the dissolution of the drug begins from each tube and the drug diffuses throughout the dressing and into the contacting tissues. As long as the dressing remains wet, the drug will continuously be delivered to contacting tissue.

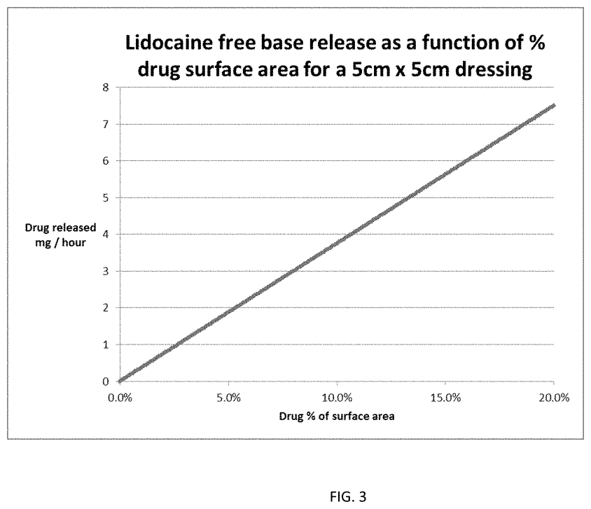

An example of the calculated delivery of the caine anesthetic from such a dressing is shown in FIG. 3. Based upon the diameter of the tube or the number of tubes used in a dressing and the solubility of the caine salt used the release rate is shown as a function of the surface area of the tube ends, that is of the total cross sectional area of both ends of the tube. This calculation assumes the drug has a dissolution constant of 1,500 micrograms per square centimeter per hour which is representative of the drug dissolution rates that can be achieved with a caine salt. The dressing size used for this calculation is 5 cm by 5 cm.

This example shows the wide range of drug delivery that is achievable with this invention showing the relationship between the cumulative surface area of exposed drug tubes and the area of the dressing or bandage.

The anesthetic tubes may also be employed in topical formulations in a variety of physical forms. For example, pastes, creams, lotions, gels, and liquids are all contemplated by the present invention. A difference between these forms is their physical appearance and viscosity, which can be governed by the presence and amount of emulsifiers and viscosity adjusters present in the formulation. Particular topical formulations can often be prepared in a variety of these forms. Solids are generally firm and will usually be in particulate form; solids optionally can contain liquids, emulsifiers, moisturizers, emollients, fragrances, dyes/colorants, preservatives and other active ingredients that increase or enhance the efficacy of the final product. Creams and lotions are often similar to one another, differing mainly in their viscosity; both lotions and creams may be opaque, translucent or clear and often contain emulsifiers, solvents, and viscosity adjusting agents, as well as moisturizers, emollients, fragrances, dyes/colorants, preservatives and other active ingredients that increase or enhance the efficacy of the final product. Gels can be prepared with a range of viscosities, from thick or high viscosity to thin or low viscosity. These formulations, like those of lotions and creams may also contain liquids, emulsifiers, moisturizers, emollients, fragrances, dyes/colorants, preservatives and other ingredients that increase or enhance the efficacy of the final product. Liquids are thinner than creams, lotions, or gels and often do not contain emulsifiers.

Applications include such examples as thermal burns, sun burns, friction burns, hemorrhoids, abrasions, lacerations, dermal penetrations and any similar injury where the treatment of pain is desired. The anesthetic agent may be combined with other active medicaments in such products such as antibiotics, antibacterials, sun screens or other ingredients that are used for the intended use of the product.

In such topical applications the anesthetic tubes are added during the application of the topical product to activate and initiate the release of the anesthetic agent. This may be accomplished in a variety of ways that allow the mixing of the drug eluting tubes into the composition. For example, the tubes may be contained in a separate compartment of a two part dispenser. A membrane separating the two components is broken by finger pressure allowing the mixing of the two components which are subsequently mixed by kneading the packaging. The product is subsequently dispensed for the intended application. In another delivery method the anesthetic tubes are contained in a nonaqueous vehicle such as propylene glycol where the solubility of the caine salt is low. This liquid is contained in a two part tube and mixing of the aqueous lotion or cream is accomplished when product is squeezed from the container. Alternatively, the anesthetic tubes are simply mixed with the product prior to administration. There are many means by which the free flowing anesthetic tubes may be combined with a topical product by those skilled in the art to achieve the activation of the anesthetic tubes and the release of the caine anesthetic.

In dressing or bandage applications the anesthetic caine tubes are integral to the manufacture of the product. The product is stored in a dry state and activated at time of use by wetting the dressing with moisture. Alternatively, the dressing may be stored pre-wetted with a nonaqueous agent such as propylene glycol. Application of this dressing to a wound will result in the absorption of water which will initiate the release of the caine anesthetic.

The caine anesthetic employed in the drug delivery tubes may be comprised of any caine that has been approved for use. Local anesthetics of the “caine” family are weak bases. (By “caine” is intended anesthetics that end in the suffix caine, which will usually include amino acid amides and esters). One of the classes of anesthetics that are amine bases also includes an aromatic ring, for example, a meta xylyl group and an amide or ester functionality. The aromatic group with the other entities results in hydrophobicity, so that the members of the class are frequently employed as their hydrochloride salts to allow for water solubility. Examples of such anesthetics of the caine family include lidocaine (lignocaine), procaine, bupivacaine, ropivacaine, butacaine, oxybuprocaine, mepivacaine, prilocaine, amylocaine, chloroprocaine, etidocaine, propoxycaine and tropacocaine.

The caines may be employed in the anesthetic tubes as their free base form, their hydrochloride form or other salts which may be employed to slow the rate of dissolution of the caine, such as those described in U.S. Pat. No. 8,920,843 B2. Generally, either the free base form or a hydrophobic salt form of the caine anesthetic would be employed in a drug delivery application if longer term delivery of the caine is desired. For shorter term delivery of the caine a more soluble salt such as the hydrochloride or similar salts of the caine anesthetic such as the hydrobromide, hydroiodide or other salts which be very soluble in water and would be known to those skilled in the art. In all cases the rate of delivery can be controlled through the selection of the exposed surface area as described previously and the release can be readily calculated knowing the unit solubility per unit area and unit of time.

In certain embodiments, the hydrophobic caine salt is present in the composition of the invention in the form of particles. The size distribution of the particles will generally have at least about 50 weight %, within 75%, more usually within 50%, and desirably within 25% of the median size. The median size will generally range from about 1 to about 2000 m, more usually from about 5 to 1500 m, desirably from about 5 m to 1200 m. Individual compositions of interest have median sizes of about 1 to 25 m; 5 to 100 m; 100 to 200 m, 300 to 500 m 500 to 750 m, 600 to 700 m and 750 to 1200 m. In one embodiment, the median size of the particles is about 625 to 675 m, or about 650 m.

Depending upon the manner in which the particles are made, they can comprise less than about 2, more usually less than about 1, weight % of the solvent used in their preparation, and preferably undetectable amounts.

The stabilizing agent is an excipient which prevents or inhibits aggregation of the particles. Suitable stabilizing agents include non-ionic surfactants, such as low molecular weight polyethylene glycols, including PEG200. In certain embodiments, the stabilizing agent is selected to be soluble in the liquid vehicle. The amount of stabilizer in the composition is preferably sufficient to substantially inhibit aggregation of the particles.

Once the particles have been prepared, irrespective of the method employed in their preparation, the particles are sized and fractioned typically by sieving operations, although other methods may be employed. To control particle distribution and particle size a typical sieving operation would employ at least 2 sieves of the appropriate size. The larger sieve size would allow for the rejection of particles larger than the specified maximum while the lower sieve size would serve to retain the particles of the specified size. The selection of the sieves determines the particle size distribution. Using this approach one can also prepare multimodal distributions to obtain different release profiles of drug. Nominal particle size and particle size distribution is determined by an instrument such as a Coulter LS13 on suspensions of the microparticles.

### VIII. Definitions

The term “alkyl” is intended to include both branched and straight chain, saturated aliphatic hydrocarbon radicals/groups having the specified number of carbons. Preferred alkyl groups comprise about 1 to about 24 carbon atoms (“C₁-C₂₄”) preferably about 7 to about 24 carbon atoms (“C₇-C₂₄”), preferably about 8 to about 24 carbon atoms (“C₈-C₂₄”), preferably about 9 to about 24 carbon atoms (“C₉-C₂₄”). Other preferred alkyl groups comprise about 1 to about 12 (“C₁-C₁₂”), about 1 to about 8 carbon atoms (“C₁-C₈”) such as about 1 to about 6 carbon atoms (“C₁-C₆”), or such as about 1 to about 3 carbon atoms (“C₁-C₃”). Examples of C₁-C₆ alkyl radicals include, but are not limited to, methyl, ethyl, propyl, isopropyl, n-butyl, tert-butyl, n-pentyl, neopentyl and n-hexyl radicals.

The term “haloalkyl” is intended to refer to an alkyl group in which at least one hydrogen atom is substituted with a halogen atom, preferably a fluorine, chlorine or bromine atom. Preferred haloalkyl groups have at least two or three halogen substituents. In a haloalkyl having two or more halogen substituents, the halogen substituents can be the same or different. A “perhaloalkyl” is a haloalkyl group in which all hydrogen atoms are substituted with halogen atoms, preferably chlorine and/or fluorine atoms. Preferably, a perhaloalkyl group is a perchloroalkyl group or a perfluoroalkyl group, more preferably a perfluoroalkyl group, such as a perfluoro-n-C₁-C₁₀-alkyl.

The term “alkenyl” refers to linear or branched radicals having at least one carbon-carbon double bond. Such radicals preferably contain from about two to about twenty-four carbon atoms (“C₂-C₂₄”) preferably about 7 to about 24 carbon atoms (“C₇-C₂₄”), preferably about 8 to about 24 carbon atoms (“C₈-C₂₄”), and preferably about 9 to about 24 carbon atoms (“C₉-C₂₄”). Other preferred alkenyl radicals are “lower alkenyl” radicals having two to about ten carbon atoms (“C₂-C₁₀”) such as ethenyl, allyl, propenyl, butenyl and 4-methylbutenyl. Preferred lower alkenyl radicals include 2 to about 6 carbon atoms (“C₂-C₆”). The terms “alkenyl”, and “lower alkenyl”, embrace radicals having “cis” and “trans” orientations, or alternatively, “E” and “Z” orientations.

The term “alkynyl” refers to linear or branched radicals having at least one carbon-carbon triple bond. Such radicals preferably contain from about two to about twenty-four carbon atoms (“C₂-C₂₄”), preferably about 7 to about 24 carbon atoms (“C₇-C₂₄”), preferably about 8 to about 24 carbon atoms (“C₈-C₂₄”), and preferably about 9 to about 24 carbon atoms (“C₉-C₂₄”). Other preferred alkynyl radicals are “lower alkynyl” radicals having two to about ten carbon atoms such as propargyl, 1-propynyl, 2-propynyl, 1-butyne, 2-butynyl and 1-pentynyl. Preferred lower alkynyl radicals include 2 to about 6 carbon atoms (“C₂-C₆”).

The term “substituted” refers to the replacement of one or more hydrogen radicals in a given structure with the radical of a specified substituent including, but not limited to: halo, alkyl, alkenyl, alkynyl, aryl, heterocyclyl, thiol, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, sulfonic acid, sulfonyl, phosphonic acid, aryl, heteroaryl, heterocyclic, and aliphatic. It is understood that the substituent may be further substituted. In embodiments in which R₁ and/or R₂ is substituted, the one or more substituents are preferably independently selected to increase the hydrophobicity of the resulting acid. In this embodiment, for example, the substituents can be halogen, such as chloro or fluoro, aryl, such as phenyl or naphthyl, or alkyl, for example, C₁-C₆-alkyl.

The term “aliphatic group” or “aliphatic” refers to a non-aromatic moiety that may be saturated (e.g. single bond) or contain one or more units of unsaturation, e.g., double and/or triple bonds. An aliphatic group may be straight chained, branched or cyclic, contain carbon, hydrogen or, optionally, one or more heteroatoms and may be substituted or unsubstituted. In addition to aliphatic hydrocarbon groups, aliphatic groups include, for example, polyalkoxyalkyls, such as polyalkylene glycols, polyamines, and polyimines, for example. Such aliphatic groups may be further substituted. It is understood that aliphatic groups may include alkyl, substituted alkyl, alkenyl, substituted alkenyl, alkynyl, substituted alkynyl, and substituted or unsubstituted cycloalkyl groups as described herein.

The term “cycloalkyl” refers to saturated carbocyclic radicals having three to about twelve carbon atoms (“C₃-C₁₂”). The term “cycloalkyl” embraces saturated carbocyclic radicals having three to about twelve carbon atoms. Examples of such radicals include cyclopropyl, cyclobutyl, cyclopentyl and cyclohexyl.

The term “cycloalkenyl” refers to partially unsaturated carbocyclic radicals having three to twelve carbon atoms. Cycloalkenyl radicals that are partially unsaturated carbocyclic radicals that contain two double bonds (that may or may not be conjugated) can be called “cycloalkyldienyl”. More preferred cycloalkenyl radicals are “lower cycloalkenyl” radicals having four to about eight carbon atoms. Examples of such radicals include cyclobutenyl, cyclopentenyl and cyclohexenyl.

The term “alkylene,” as used herein, refers to a divalent group derived from a straight chain or branched saturated hydrocarbon chain having the specified number of carbons atoms. Examples of alkylene groups include, but are not limited to, ethylene, propylene, butylene, 3-methyl-pentylene, and 5-ethyl-hexylene.

The term “alkenylene,” as used herein, denotes a divalent group derived from a straight chain or branched hydrocarbon moiety containing the specified number of carbon atoms having at least one carbon-carbon double bond. Alkenylene groups include, but are not limited to, for example, ethenylene, 2-propenylene, 2-butenylene, 1-methyl-2-buten-1-ylene, and the like.

The term “alkynylene,” as used herein, denotes a divalent group derived from a straight chain or branched hydrocarbon moiety containing the specified number of carbon atoms having at least one carbon-carbon triple bond. Representative alkynylene groups include, but are not limited to, for example, propynylene, 1-butynylene, 2-methyl-3-hexynylene, and the like.

The term “alkoxy” refers to linear or branched oxy-containing radicals each having alkyl portions of one to about twenty-four carbon atoms or, preferably, one to about twelve carbon atoms. More preferred alkoxy radicals are “lower alkoxy” radicals having one to about ten carbon atoms and more preferably having one to about eight carbon atoms. Examples of such radicals include methoxy, ethoxy, propoxy, butoxy and tert-butoxy.

The term “alkoxyalkyl” refers to alkyl radicals having one or more alkoxy radicals attached to the alkyl radical, that is, to form monoalkoxyalkyl and dialkoxyalkyl radicals.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indanyl, indenyl, biphenyl, anthracenyl and phenanthrenyl. A preferred aryl group is phenyl.

The terms “heterocyclyl”, “heterocycle” “heterocyclic” or “heterocyclo” refer to saturated, partially unsaturated and unsaturated heteroatom-containing ring-shaped radicals, which can also be called “heterocyclyl”, “heterocycloalkenyl” and “heteroaryl” correspondingly, where the heteroatoms may be selected from nitrogen, sulfur and oxygen. Examples of saturated heterocyclyl radicals include saturated 3 to 6-membered heteromonocyclic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole. Heterocyclyl radicals may include a pentavalent nitrogen, such as in tetrazolium and pyridinium radicals. The term “heterocycle” also embraces radicals where heterocyclyl radicals are fused with aryl or cycloalkyl radicals. Examples of such fused bicyclic radicals include benzofuran, benzothiophene, and the like.

The term “heteroaryl” refers to unsaturated aromatic heterocyclyl radicals. Examples of heteroaryl radicals include unsaturated 3- to 6-membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.), tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3- to 6-membered heteromonocyclic group containing an oxygen atom, for example, pyranyl, furyl, etc.; unsaturated 3- to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3- to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like.

The term “heterocyclylalkyl” refers to heterocyclo-substituted alkyl radicals. More preferred heterocycloalkyl radicals are “lower heterocycloalkyl” radicals having one to six carbon atoms in the heterocyclo radical.

The term “alkylthio” refers to radicals containing a linear or branched alkyl radical, of one to about ten carbon atoms attached to a divalent sulfur atom. Preferred alkylthio radicals have alkyl radicals of one to about twenty-four carbon atoms or, preferably, one to about twelve carbon atoms. More preferred alkylthio radicals have alkyl radicals which are “lower alkylthio” radicals having one to about ten carbon atoms. Most preferred are alkylthio radicals having lower alkyl radicals of one to about eight carbon atoms. Examples of such lower alkylthio radicals include methylthio, ethylthio, propylthio, butylthio and hexylthio.

The terms “aralkyl” or “arylalkyl” refer to aryl-substituted alkyl radicals such as benzyl, diphenylmethyl, triphenylmethyl, phenylethyl, and diphenylethyl.

The term “aryloxy” refers to aryl radicals attached through an oxygen atom to other radicals.

The terms “aralkoxy” or “arylalkoxy” refer to aralkyl radicals attached through an oxygen atom to other radicals.

The term “aminoalkyl” refers to alkyl radicals substituted with amino radicals. Preferred aminoalkyl radicals have alkyl radicals having about one to about twenty-four carbon atoms or, preferably, one to about twelve carbon atoms. More preferred aminoalkyl radicals are “lower aminoalkyl” that have alkyl radicals having one to about ten carbon atoms. Most preferred are aminoalkyl radicals having lower alkyl radicals having one to eight carbon atoms. Examples of such radicals include aminomethyl, aminoethyl, and the like.

The term “alkylamino” denotes amino groups which are substituted with one or two alkyl radicals. Preferred alkylamino radicals have alkyl radicals having about one to about twenty carbon atoms or, preferably, one to about twelve carbon atoms. More preferred alkylamino radicals are “lower alkylamino” that have alkyl radicals having one to about ten carbon atoms. Most preferred are alkylamino radicals having lower alkyl radicals having one to about eight carbon atoms. Suitable lower alkylamino may be monosubstituted N-alkylamino or disubstituted N,N-alkylamino, such as N-methylamino, N-ethylamino, N,N-dimethylamino, N,N-diethylamino or the like.

For simplicity, chemical moieties that are defined and referred to throughout can be univalent chemical moieties (e.g., alkyl, aryl, etc.) or multivalent moieties under the appropriate structural circumstances clear to those skilled in the art. For example, an “alkyl” moiety can be referred to a monovalent radical (e.g. CH₃—CH₂—), or in other instances, a bivalent linking moiety can be “alkyl,” in which case those skilled in the art will understand the alkyl to be a divalent radical (e.g., —CH₂—CH₂—), which is equivalent to the term “alkylene.” Similarly, in circumstances in which divalent moieties are required and are stated as being “alkoxy”, “alkylamino”, “aryloxy”, “alkylthio”, “aryl”, “heteroaryl”, “heterocyclic”, “alkyl” “alkenyl”, “alkynyl”, “aliphatic”, or “cycloalkyl”, those skilled in the art will understand that the terms alkoxy”, “alkylamino”, “aryloxy”, “alkylthio”, “aryl”, “heteroaryl”, “heterocyclic”, “alkyl”, “alkenyl”, “alkynyl”, “aliphatic”, or “cycloalkyl” refer to the corresponding divalent moiety.

The terms “halogen” or “halo” as used herein, refers to an atom selected from fluorine, chlorine, bromine and iodine.

By a “therapeutically effective amount” of a caine compound of the invention is meant an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect).

“Treatment” or “treating” refers to an approach for obtaining beneficial or desired clinical results in a patient. For purposes of this invention, beneficial or desired clinical results include, but are not limited to, one or more of the following: alleviation of symptoms, diminishment of extent of a disease, stabilization (i.e., not worsening) of a state of disease, preventing spread (i.e., metastasis) of disease, preventing occurrence or recurrence of disease, delay or slowing of disease progression, amelioration of the disease state, and remission (whether partial or total).

The term “acidic therapeutic agent”, which is used interchangeably herein with the term “acidic drug”, refers to a drug which contains one or more acidic functional groups. Acid therapeutic agents include monoacidic therapeutic agents, which contain only one acidic functional group under the conditions of salt formation, and polyacidic therapeutic agents, which contain at least two such functional groups. Acidic functional groups include, but are not limited to, carboxylic acid, sulfonic acid, tetrazole, sulfonamide, urea, sulfonyl urea, phosphonic acid and imide groups.

The term “sustained release” as used herein, means that administration of an acid addition salt of a parent drug of the invention to a subject results in effective systemic, local or plasma levels of the parent drug in the subject's body for a period of time that is longer than that resulting from administration of the parent drug which is not formulated with the acid addition salt of the present invention.

The term “basic therapeutic agent”, which is used interchangeably herein with the term “basic drug”, refers to a drug which contains one or more basic functional groups. Basic therapeutic agents include monobasic therapeutic agents, which contain only one basic functional group under the conditions of salt formation, and polybasic therapeutic agents, which contain at least two such functional groups. Basic functional groups include primary, secondary, tertiary and quaternary amino groups, amidino groups, imino groups, guanidino groups and basic N-containing heteroaryl groups.

### IX. Pharmaceutical Compositions

The pharmaceutical compositions of the present invention comprise a therapeutically effective amount of an acid addition salt of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; cyclodextrins such as alpha-(α), beta- (β) and gamma- (γ) cyclodextrins; starches such as corn starch and potato starch; cellulose and its derivatives such as sodium carboxymethylcellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In certain embodiments, the formulations include a viscoelastic polymer, such as hyaluronic acid, chondroitin sulfate or a glycosaminoglycan. In other embodiments, the formulations include a water soluble low molecular weight polymer, such as polyethylene glycol.

The pharmaceutical compositions of this invention may be administered orally, parenterally, by inhalation spray, topically, rectally, nasally, buccally, vaginally or via an implanted reservoir. In a preferred embodiment, administration is parenteral administration by injection.

The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intracisternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, dimethylacetamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable suspension or emulsion, such as INTRALIPID®, LIPOSYN® or OMEGAVEN®, or solution, in a nontoxic parenterally acceptable diluent or solvent, for example, as a solution in 1,3-butanediol. INTRALIPID® is an intravenous fat emulsion containing 10-30% soybean oil, 1-10% egg yolk phospholipids, 1-10% glycerin and water. LIPOSYN® is also an intravenous fat emulsion containing 2-15% safflower oil, 2-15% soybean oil, 0.5-5% egg phosphatides 1-10% glycerin and water. OMEGAVEN® is an emulsion for infusion containing about 5-25% fish oil, 0.5-10% egg phosphatides, 1-10% glycerin and water. Among the acceptable vehicles and solvents that may be employed are water, Ringer's solution, USP and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The formulations can also be sterilized by other methods, including heat and/or radiation, such as gamma, ultraviolet or electron beam radiation.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to VanDevanter et al., U.S. Pat. No. 5,508,269 to Smith et al., and WO 98/43650 by Montgomery, all of which are incorporated herein by reference). A discussion of pulmonary delivery of antibiotics is also found in U.S. Pat. No. 6,014,969, incorporated herein by reference.

In preferred embodiments, the compounds of the invention, or pharmaceutical compositions comprising one or more compounds of the invention, are administered parenterally, for example, by intramuscular, subcutaneous or intraperitoneal injection. Without being bound by theory, it is believed that upon injection, compounds of the invention form an insoluble or sparingly soluble depot from which drug molecules are released over time.

In certain embodiments, the salts of the invention, such as caine salts, are present in the composition in the form of particles.

The compounds and processes of the present invention will be better understood in connection with the following synthetic schemes that illustrate the methods by which the compounds of the invention may be prepared, which are intended as an illustration only and not to limit the scope of the invention. Various changes and modifications to the disclosed embodiments will be apparent to those skilled in the art and such changes and modifications including, without limitation, those relating to the chemical structures, substituents, derivatives, and/or methods of the invention may be made without departing from the spirit of the invention and the scope of the appended claims.

### EXAMPLES

### Example 1 Synthesis of Compound 7

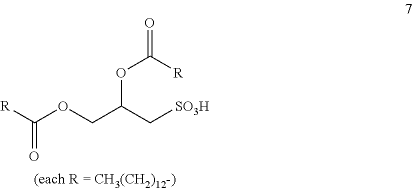

### (a) Preparation of Compound 1

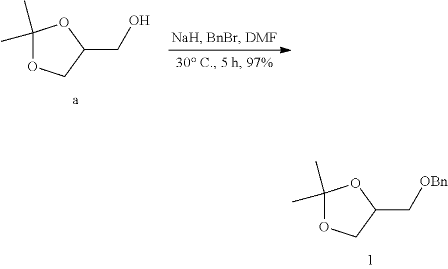

To a mixture of compound a (50.0 g, 378.3 mmol, 1.0 eq) in DMF (450 mL) was added NaH (16 g, 666.7 mmol, 1.7 eq) and BnBr (64.7 g, 378.3 mmol, 1.0 eq) at 0° C. The reaction was stirred at 30° C. for 5 h under nitrogen atmosphere. Then the mixture was diluted with ethyl acetate and water, separated and the organic layer was washed with brine, dried over Na₂SO₄, concentrated to give compound 1 (82.2 g, 97%), which was used for next step without further purification.

### (b) Preparation of Compound 2

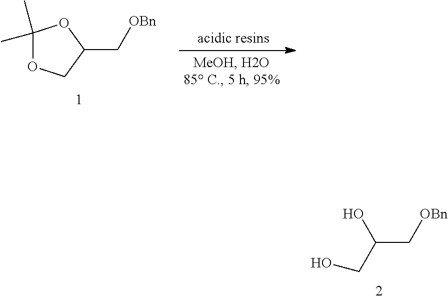 [SMILES: CC1(C)OCC(COCc2ccccc2)O1 ; OCC(O)COCc1ccccc1]

To a solution of compound 1 (82.2 g, 369.1 mmol, 1.0 eq) in MeOH/H₂O (140 mL/140 mL) was added acidic resins (82.5 g). The reaction was stirred at 85° C. for 5 h. Then the reaction was filtered and the filtrate was concentrated to give compound 2 (64.5 g, 95%), which was used for next step without further purification.
(c) Preparation of Compound 3

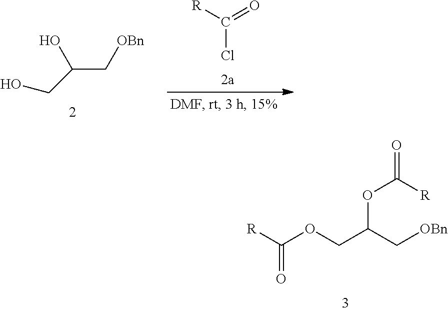

To a solution of compound 2 (65.3 g, 358.6 mmol, 1.0 eq) in DMF (950 mL) at 0° C. was added compound 2a (221.3 g, 896.6 mmol, 2.5 eq) dropwise. The reaction was stirred at rt for 3 h. Then the reaction was diluted with water and extracted with ethyl acetate. The organic layer was washed with brine, dried over sodium sulfate, concentrated to give a residue, which was purified by column chromatography purification (ethyl acetate/petroleum ether, 1:50) to afford compound 3 (32.2 g, 15%).

### (d) Preparation of Compound 4

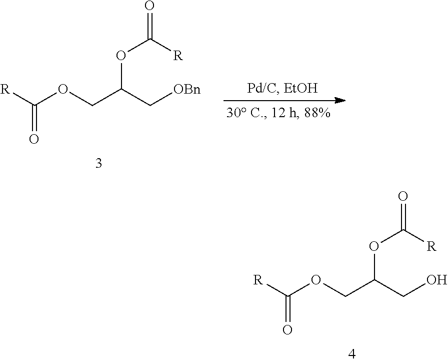

To a solution of compound 3 (20.0 g, 33.1 mmol, 1.0 eq) in EtOH (500 mL) was added Pd/C (17.6 g) and the mixture was stirred under hydrogen atmosphere at 30° C. for 12 h. The reaction was filtered and filtrated was concentrated to give compound 4 (15.0 g, 88%).

### (e) Preparation of Compound 5

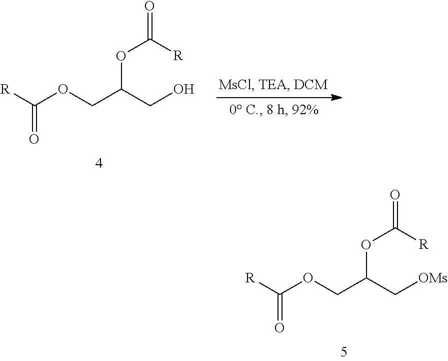

To a mixture of compound 4 (17.0 g, 33.2 mmol, 1.0 eq) and triethylamine (4.1 g, 44.5 mmol, 1.2 eq) in dry DCM (200 mL) at 0° C. was added mesyl chloride (4.2 g, 36.4 mmol, 1.1 eq). The resulting mixture was stirred at 0° C. for 8 h, and extracted with ethyl acetate. The organic layer was dried over Na₂SO₄, and concentrated to give crude compound 5 (18.2 g, 92%).

### (f) Preparation of Compound 6

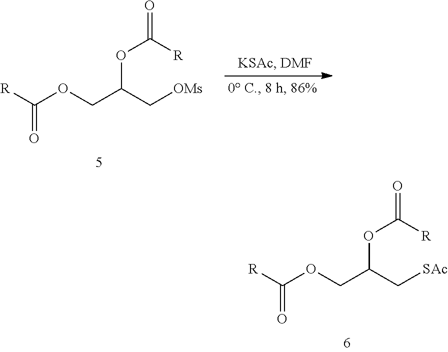

To a solution of compound 5 (18.0 g, 30.5 mmol, 1.0 eq) in DMF (150 mL) was added potassium thioacetate (17.4 g, 152.5 mol, 5.0 eq). The mixture was stirred at 0° C. for 8 h. The mixture was extracted with ethyl acetate. The organic layer was washed with brine, dried over Na₂SO₄, concentrated to give a crude residue, which was purified by column chromatography purification (ethyl acetate/petroleum ether, 1:20) to afford compound 6 (15.2 g, 86%).

### (g) Preparation of Compound 7

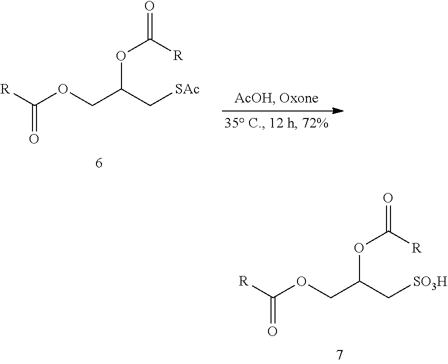

To a solution of compound 6 (9.0 g, 15.7 mmol, 1.0 eq) in acetic acid (150 mL) was added oxone (14.6 g, 47.3 mmol, 3.0 eq). The mixture was stirred at 35° C. for 12 h. The mixture was diluted with water (200 mL) and stirred for 1 h. The solid was filtered and washed with water (3×100 mL), and dried in vacuo. The solid was further triturated with ether to give compound 7 (6.5 g, 72%).

¹H NMR (400 MHz, DMSO-d₆) δ 5.27-5.22 (m, 1H), 4.45-4.42 (m, 1H), 4.07-4.04 (m, 1H), 2.74-2.63 (m, 3H), 2.20-2.16 (m, 6H), 1.46-1.44 (m, 6H), 1.25-1.22 (m, 45H), 0.81-0.79 (m, 9H).

### Example 2 Synthesis of the Pyridinium Salt of Compound 8 (Compound 9)

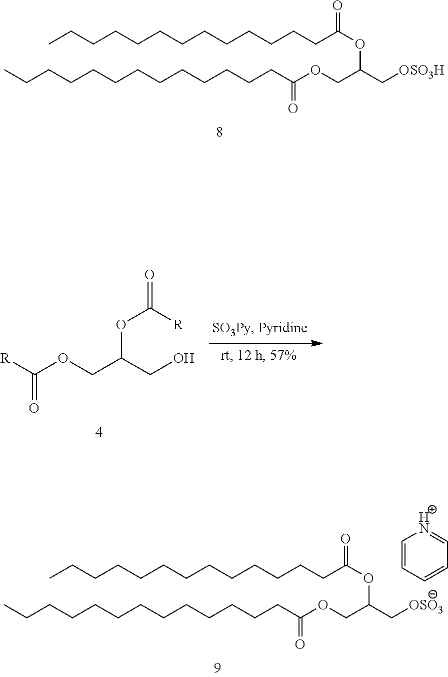

To a solution of compound 4 (4.0 g, 7.8 mmol, 1.0 eq) in pyridine (100 mL) at 0° C. was added sulfur trioxide pyridine complex (3.8 g, 23.4 mmol, 3.0 eq). The mixture was stirred at rt for 12 h, filtered. The filtrate was concentrated in vacuo to give a crude residue, which was triturated with ethyl acetate and dried in vacuo to give compound 9 (6.0 g, 57%) as a white solid.

¹H NMR (400 MHz, DMSO-d₆) δ 8.94 (d, J=8.0 Hz, 2H), 8.46 (t, J=7.6 Hz, 1H), 8.11 (t, J=8.0 Hz, 2H), 5.29-5.26 (m, 1H), 4.45-4.40 (m, 1H), 4.21-4.15 (m, 3H), 2.26-2.23 (m, 4H), 1.59-1.50 (m, 4H), 1.26-1.19 (m, 46H), 0.81-0.79 (m, 6H).

### Example 3 Preparation of the Lidocaine Salt of Compound 8 (Compound 10)

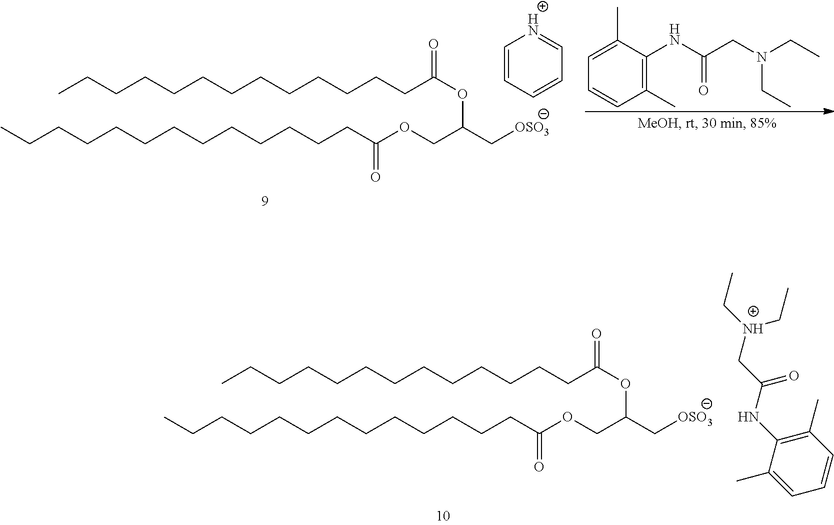

Into a solution of compound 9 (1.0 g, 1.48 mmol, 1.0 eq) in methanol (90 mL) was slowly added lidocaine (349 mg, 1.48 mmol, 1.0 eq) in methanol (10 mL). The mixture was stirred at rt for 30 min and then concentrated. The residue was diluted with ethyl acetate (100 mL) and washed with water and brine, dried over sodium sulfate and concentrated in vacuo to give a crude product, which was triturated with ethyl acetate, and dried to give compound 10 (1.05 g, 85%) as a white solid.

¹H NMR (400 MHz, CDCl₃) δ 7.04-6.99 (m, 3H), 5.29-5.27 (m, 1H), 4.34-4.30 (m, 3H), 4.11-4.07 (m, 3H), 3.48-3.32 (m, 4H), 2.29-2.24 (m, 4H), 2.18 (s, 6H), 1.54-1.50 (m, 4H), 1.40-1.37 (m, 7H), 1.25-1.22 (m, 45H), 0.87 (t, J=6.4 Hz, 6H).

### Example 4—Pharmacokinetics of Caine Anesthetic Salts in Female Sprague Dawley Rats after a Single Subcutaneous Dose

Test Article: Compound 10, Nominal Particle Size 325 μm at 289 mg/kg

This study was designed to evaluate the pharmacokinetic consequences of using dry drug particles (powder) subcutaneously in the rat. The test article was provided as a powder and was administered by single subcutaneous dose via incision to five female Sprague Dawley (CD) rats. Each animal received a single subcutaneous dose of the test article delivered to the dorsal subcutaneum of the animals, via a dorsal skin incision. On the day of testing, each animal's dose was individually weighed and recorded. After surgical preparation of an animal, an incision was made in the dorsum, near the scapular region, and the test article was carefully deposited into the subcutaneum on the exposed area. The incision was closed, animal provided analgesic and antibiotic therapies and allowed to recover. This process was repeated for all animals on study. Cage side observations were performed daily, and blood samples for pharmacokinetic (PK) analysis were collected at pre-dose and at 1, 4, 8, 24, 48, 72, and 96 hours post-dose, processed to plasma and stored frozen at −60 to −80° C. until sent for analysis. In addition, at study termination on Day 4 (96 hours post dose administration), animals were euthanized, and the dose sites were exposed for gross evaluation of residual test articles and local response to the injected materials. The subcutaneous tissues surrounding the dose sites were collected and frozen at −60 to −80° C. for bioanalysis.

All animals survived the study period and appeared healthy. Slight body weight loss (less than 5%) was observed in several animals at the end of the study, but was present in all groups. Body weight loss may have been exacerbated by the surgical dose administration and repeated blood collection. Edema was present at the dose sites at the end of the study period on Day 4. After euthanasia, the dose site observations included moderate to severe vascularization surrounding the incision sites on the subcutaneous skin layers. For all animals, it appeared that all test article remained as implanted and was encapsulated. Yellowish fluid was present within all tissue capsules. Hematoma was present under the incision site in one animal.

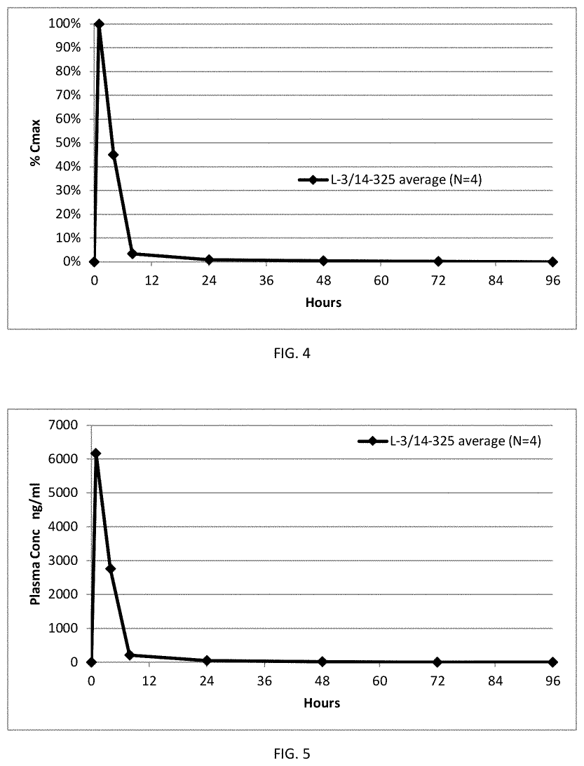

The pharmacokinetic results of this study are shown in FIGS. 4 and 5. Subcutaneous adminstration of compound 10 in the form of dry particles results in measurable plasma levels oflidocaine over 24 hours.

### Example 5—Preparation of of Tridecafluorohexane-1-Sulfonate Salts

### (a) Preparation of Tridecafluorohexane-1-Sulfonic Acid

$${C}_{6} {F}_{13} {SO}_{3} K {\to }_{ acetone }^{HCl} {C}_{6} {F}_{13} {SO}_{3} H$$

To a solution of potassium tridecafluorohexane-1-Sulfonate (15.3 g, 30.0 mmol, 1.0 eq) in acetone (300 mL) was added HCl (37%, 2.25 mL, 33.0 mmol, 1.1 eq) at room temperature. The reaction was stirred at room temperature 1 h. The mixture was filtered and concentrated to dryness. The residue was diluted with ethyl acetate and washed with water. The organic phase was separated, dried with sodium sulfate, and concentrated to afford tridecafluorohexane-1-sulfonic acid (14.2 g) as a white solid.

### (b) Preparation of Lidocaine Tridecafluorohexane-1-Sulfonate Salt

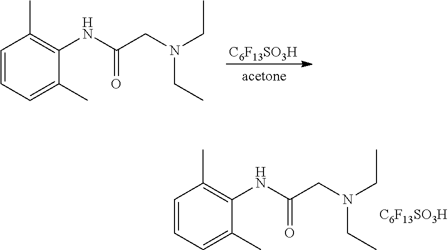

To a solution of lidocaine (5.85 g, 25.0 mmol, 1.0 eq) in acetone (100 mL) was added tridecafluorohexane-1-sulfonic acid (10.0 g, 25.0 mmol, 1.0 eq) at room temperature. The reaction was stirred for 1 h. The mixture was concentrated to dryness. The residue was recrystallized with acetone/diethyl ether. The precipitate was filtered and dried to give lidocaine tridecafluorohexane-1-sulfonate salt (12.6 g) as a white solid.

### (c) Preparation of Bupivacaine Free Base

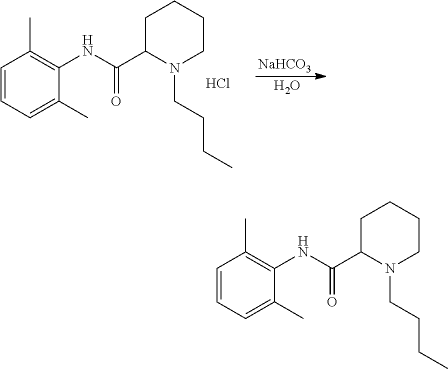

To a solution of bupivacaine hydrochloride (25.0 g, 77.1 mmol, 1.0 eq) in acetone (100 mL) at room temperature was added saturated NaHCO₃ solution to adjust the solution to pH 7. The mixture was diluted with ethyl acetate and washed with water. The organic phase was separated, dried over sodium sulfate, and concentrated to afford bupivacaine free base (23.9 g) as a white solid.

### (d) Preparation of Bupivacaine Tridecafluorohexane-1-Sulfonate Salt

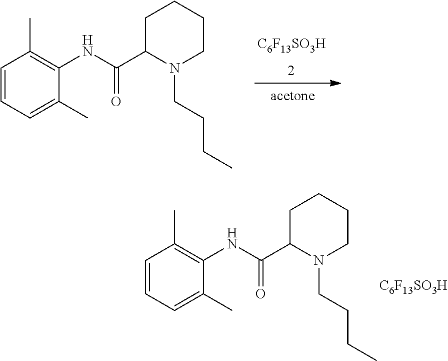

To a solution of bupivacaine free base (7.68 g, 26.6 mmol, 1.0 eq) in acetone (100 mL) was added tridecafluorohexane-1-sulfonic acid (13.3 g, 26.6 mmol, 1.0 eq) at room temperature. The reaction was stirred for 1 h. The mixture was concentrated to dryness. The residue was triturated with ethyl ether/hexanes to afford the title compound (14.0 g) as white solid. The solid (14.0 g) was recrystallized with acetone/diethyl ether. The precipitate was filtered and dried in vacuo to give bupivacaine tridecafluorohexane-1-sulfonate salt (10.6 g) as a white solid.

### (e) Synthesis of Bupivacaine Heptadecafluorooctane-1-Sulfonate Salt

Potassium heptadecafluorooctane-1-sulfonate (19.8 mg; 0.037 mmol) was dissolved in a minimal amount of acetone. This solution was added to a solution of bupivacaine hydrochloride (12.8 mg; 0.039 mmol) in water. The acetone was removed from the resulting solution by heating. The product precipitated as a solid and was isolated by vacuum filtration and dried.

Lidocaine heptadecafluorooctane-1-sulfonate salt was prepared in the same manner.

### (f) Preparation of Particles

Bupivacaine tridecafluorohexane-1-sulfonate (5 g) was placed into a 250-ml glass vessel. Air was evacuated from the vessel and displaced with nitrogen; the container was then placed into an oil bath preheated to 150° C. The bupivacaine salt was incubated under nitrogen flow at 150° C. for approximately 5 minutes. The resulting melt was cooled to ambient temperature under nitrogen. The resulting solids were removed from the container, crushed using mortar and pestle and fractionated on an 8″-diameter stainless steel sieve set. The fractions of the product retained between the sieve pairs with mesh size 20/25, 30/35, 35/40, 45/50 and 60/70 (corresponding to an average particle size 650, 550, 460, 325 and 230 micron, respectively) were retained for evaluation.

### Example 6 Preparation of Bupivacaine 4-Bromobenzenesulfonate Salt

To a solution of 4-bromo-benzenesulfonic acid (2.55 g, 10.0 mmol) in ether (50 mL) and acetone (5 mL) at rt was added a solution of bupivacaine (2.95 g, 10.2 mmol) in ether (20 mL) and acetone (5 ml). The reaction mixture stirred overnight, the solid was filtered and washed with ether (2×), and dried in vacuo to give the desired salt (4.3 g, 82%) as a white solid. 1H NMR (300 MHz, CD₃OD) δ 7.72-7.69 (d, J=7.8 Hz, 2H), 7.58-7.56 (d, J=8.1 Hz, 2H), 7.16-7.10 (m, 3H), 4.10-4.06 (d, J=10.2 Hz, 1H), 3.70-3.65 (d, J=12.6 Hz, 1H), 3.17-3.07 (t, J=8.4 Hz, 3H), 2.42-2.36 (d, J=11.7 Hz, 1H), 2.21 (s, 7H), 1.99-1.71 (m, 8H), 1.46-1.33 (m, 2H), 1.00-0.95 (t, J=7.5 Hz, 3H).

### Example 7 Preparation of Bupivacaine 3,4-Dibromobenzenesulfonate Salt

### (a) 3,4-Dibromobenzenesulfonic Acid

A mixture of 3,4-dibromobenzene sulfonyl chloride (4.90 g, 14.6 mmol) in dioxane/water (20/10 mL) was heated to 100° C. for 6 h. The reaction was cooled to room temperature and lyophilized to afford 3,4-dibromobenzenesulfonic acid the title compound (4.68 g, 99%). LC-MS: 314.7 [M-H]; 1H NMR (300 MHz, DMSO-d6) δ 7.81 (s, 1H), 7.75 (d, J=7.8 Hz, 1H), 7.43 (d, J=7.8 Hz, 1H).

### (b) Bupivacaine 3,4-Dibromobenzenesulfonate Salt

A solution of 3,4-dibromobenzenesulfonic acid (1.6 g, 5.06 mmol) and bupivacaine (1.6 g, 5.55 mmol) in acetone (5 mL) and ether (40 mL) was stirred overnight. The oil at the bottom of the reaction vessel solidified slowly. The solvent was removed, and the solid was triturated with ether (2×) and dried to yield bupivacaine 3,4-dibromobenzenesulfonate salt (2.47 g, 81%) as a white solid. ¹H NMR (300 MHz, CD₃OD) δ 8.06-8.05 (d, J=1.8 Hz, 1H), 7.75-7.72 (d, J=8.4 Hz, 1H), 7.66-7.62 (dd, J=1.8, 2.1 Hz, 1H), 7.17-7.09 (m, 3H), 4.10-4.06 (d, J=11.7 Hz, 1H), 3.70-3.66 (d, J=12.6 Hz, 1H), 3.17-3.07 (t, J=8.4 Hz, 1H), 2.42-2.36 (d, J=9.3 Hz, 1H), 2.21 (s, 6H), 1.99-1.46 (m, 9H), 1.38-1.33 (m, 2H), 1.00-0.95 (t, J=7.5 Hz, 3H).

### Example 8 Preparation of Bupivacaine 2,4-Dibromo-3-methylbenzenesulfonate Salt

(a) 2,4-Dibromo-3-methylbenzenesulfonic Acid

To a solution of 2,6-dibromotoluene (10.0 g, 40 mmol) in 1,2-dichloroethane (40 mL) was added dropwise chlorosufonic acid (4.66 g, 40 mmol, 1 eq). The reaction mixture was stirred at room temperature for 3 days, and monitored by TLC and LC-MS. After concentrated to about 20 mL, solid precipitated out. The solid was filtered and washed with hexane, and dried in vacuo to yield 2,4-dibromo-3-methylbenzenesulfonic acid (3.65 g, 28%) as pale white solid. LC-MS: 328.7 [M-H]; H NMR (300 MHz, CD₃OD): δ 7.83 (d, J=9.0 Hz, 1H), 7.62 (d, J=9.0 Hz, 1H), 2.66 (s, 3H).

(b) Bupivacaine 2,4-Dibromo-3-methylbenzenesulfonate Salt

A solution of 2,4-dibromo-3-methylbenzenesulfonic acid (2.29 g, 6.94 mmol) and bupivacaine (2.5 g, 8.68 mmol) in acetone (5 mL) and ether (40 mL) was stirred overnight. Solid formed was filtered and washed with ether, dried in vacuo to afford bupivacaine 2,4-dibromo-3-methylbenzenesulfonate salt (2.55 g, 59%) as pale white solid. ¹H NMR (300 MHz, CD₃OD): δ 7.83-7.80 (d, J=7.5 Hz, 1H), 7.61-7.58 (d, J=8.1 Hz, 1H), 7.18-7.09 (m, 3H), 4.18-4.12 (dd, J=3.6, 4.2 Hz, 1H), 3.70-3.65 (d, J=12.9 Hz, 1H), 3.19-3.10 (m, 3H), 2.64-2.63 (m, 2H), 2.42-2.38 (d, J=11.1 Hz, 1H), 2.20 (s, 7H), 1.99-1.69 (m, 9H), 1.45-1.33 (m, 2H), 1.00-0.95 (t, J=7.5 Hz, 3H).

### Example 9 Preparation of Lidocaine 4-Tetradecylbenzenesulfonate Salt

### (a) 4-Tetradecylbenzenesulfonic Acid

To a solution of tetradecylbenzene (10.0 g, 36.4 mmol) in 1,2-dichloroethane (40 mL) was added dropwise a solution of chlorosulfonic acid (4.24 g, 36.4 mmol) in 1,2-dichloroethane (10 mL). The deep brown solution was stirred at room temperature while solid started to precipitate after 30 minutes. After stirring overnight, the solvent was removed to give a brownish crude product. Recrystallization in toluene (˜50 mL) afforded 4-tetradecyl-benzenesulfonic acid (9.6 g, 76%) as a white solid. ¹H NMR (300 MHz, CDCl₃) δ 8.31 (brs, 3H), 7.74 (d, J=7.2 Hz, 2H), 7.20 (d, J=7.2 Hz, 2H), 2.61 (t, J=7.5 Hz, 2H), 1.57 (m, 2H), 1.25 (m, 22H), 0.88 (t, J=7.5 Hz, 3H).
(b) Lidocaine 4-tetradecylbenzenesulfonate salt A mixture of 4-tetradecylbenzenesulfonic acid (4.0 g, 11.3 mmol) and lidocaine (2.93 g, 12.5 mmol) in isopropanol (40 mL) was heated to form a clear solution. The solution was cooled to room temperature and stood overnight. The solid was filtered and washed with isopropanol (10 mL), and ether (2×20 mL), and dried to give lidocaine 4-tetradecylbenzenesulfonate salt (6.0 g, 90%) as white solid. ¹H NMR (300 MHz, CD₃OD) δ 7.73-7.70 (d, J=8.1 Hz, 2H), 7.24-7.22 (d, J=7.8 Hz, 2H), 7.12 (m, 3H), 4.89-4.87 (m, 2H), 4.22 (s, 2H), 3.36-3.34 (m, 3H), 2.67-2.61 (t, J=7.8 Hz, 2H), 2.23 (s, 4H), 1.61 (m, 2H), 1.40-1.35 (t, J=7.5 Hz, 6H), 1.32-1.23 (m, 16H), 0.92-0.87 (m, 6H).

### Example 10 Preparation of Lidocaine 4-Octadecylbenzenesulfonate Salt

### (a) 4-Octadecylbenzenesulfonic Acid

To a solution of n-octadecylbenzene (5.0 g, 15.1 mmol) in 1,2-dichloroethane (20 mL) was added dropwise a solution of chlorosulfonic acid (1.76 g, 15.1 mmol) in 1,2-dichloroethane (10 mL). The resulting deep brown solution was stirred at room temperature while solid started to precipitate out after 30 minutes. After stirring overnight, the solvent was removed in vacuum to give a brownish crude product. Recrystallization of the solid in toluene (˜50 mL) afforded 4-octadecylbenzenesulfonic acid (3.0 g, 48%) as a light grey solid. LC-MS: 408.9 [M-H]⁻

¹H NMR (300 MHz, CD₃OD) 7.72 (d, J=8.4 Hz, 2H), 7.18 (d, J=8.4 Hz, 2H), 2.59 (t, J=7.5 Hz, 2H), 1.57 (m, 2H), 1.25 (m, 30H), 0.88 (t, J=7.5 Hz, 3H).

### (b) Lidocaine 4-Octadecylbenzenesulfonate Salt

A mixture of 4-octadecylbenzenesulfonic acid (4.0 g, 9.7 mmol) and lidocaine (2.5 g, 10.7 mmol) in ethanol (50 mL) and water (25 mL) was heated to form a clear solution. The solution was cooled to room temperature and stood overnight. The solid was filtered and washed with isopropanol (2×10 mL), and dried for three days to give lidocaine 4-octadecyl-benzenesulfonate salt (5.5 g, 87%) as a white solid. ¹H NMR (300 MHz, CD₃OD) δ 7.73-7.70 (d, J=8.4 Hz, 2H), 7.24-7.22 (d, J=8.1 Hz, 2H), 7.14-7.10 (m, 3H), 4.89-4.87 (m, 2H), 4.22 (s, 2H), 3.36-3.34 (m, 3H), 2.66-2.61 (t, J=7.5 Hz, 2H), 2.23 (s, 4H), 1.61 (m, 2H), 1.40-1.35 (t, J=7.5 Hz, 6H), 1.32-1.23 (m, 24H), 0.92-0.87 (m, 6H).

### Example 11 Preparation of Lidocaine 4-Docosanylbenzenesulfonate Salt

### (a) 4-Docosanylbenzenesulfonic Acid

To a solution of docosanylbenzene (11.5 g, 30.0 mmol) in 1,2-dichloroethane (80 mL) was added dropwise a solution of chlorosulfonic acid (3.51 g, 30 mmol) in 1,2-dichloroethane (20 mL). After stirring overnight, the solid was filtered and washed with hexane (3×), and recrystallized from toluene, and dried to give 4-docosanylbenzenesulfonic acid (5.1 g, 36%) as a light grey solid. ¹H NMR (300 MHz, CD₃OD) δ 10.37 (brs, 3H) 7.82 (d, J=8.4 Hz, 2H), 7.34 (d, J=8.1 Hz, 2H), 2.68 (t, J=7.8 Hz, 2H), 1.61 (m, 2H), 1.40-1.10 (m, 33H), 0.88 (t, J=6.9 Hz, 3H).

### (b) Lidocaine 4-Docosanylbenzenesulfonate Salt

A mixture of 4-docosanylbenzenesulfonic acid (3.0 g, 6.4 mmol) and lidocaine (1.66 g, 7.1 mmol) in ethanol (30 mL) was heated to form a clear solution. The solution was cooled to room temperature, and stood overnight. The solid was filtered and washed with isopropanol (2×15 mL) and dried to give lidocaine 4-docosanylbenzenesulfonate salt (3.2 g, 71%) as a light grey solid. ¹H NMR (300 MHz, CD₃OD) δ 7.73-7.70 (m, 2H), 7.24-7.10 (m, 5H), 4.89-4.87 (m, 2H), 4.22 (s, 2H), 3.36-3.34 (m, 3H), 2.66-2.61 (m, 2H), 2.23 (s, 4H), 1.61 (m, 2H), 1.40-1.23 (m, 38H), 0.92-0.87 (m, 6H).

### Example 12 Preparation of Model Polymeric Films Comprising Particles

To demonstrate the ability of PEG1000 to form particle containing films, PEG1000/disodium hydrogen phosphate decahydrate Na₂HAPO₄.10H₂O compositions containing 20% wt/wt and 30% wt/wt of sodium phosphate particles with size 100-250 microns were prepared. PEG1000 (20 g) was placed in a 50-ml glass container and melted in a water bath preheated to 50° C. To prepare 20% and 30% particle containing PEG1000 compositions, melted PEG1000 (5 g) was combined with the appropriate amount of phosphate (see Table 1). The compositions were mixed thoroughly by spatula, and composition temperature was maintained at 50° C. before film forming.

Particle containing PEG1000 films, approximately 1″×4″ in size, were formed by dispersing the liquid PEG1000 compositions on the surface of polyethylene film (PE film, thickness—2 mils) with a flat stainless steel bar. The thickness of the PEG1000 films was maintained by using two spacers (thickness 15 mils or 20 mils) supporting flat bar. The temperature of PEG1000 composition was brought to ambient and the surface of the solidified films was covered doubled with a protective layer of 2 mil thick PE film. The film was easily detached from the PE protective film. The estimated PEG1000 film phosphate particle content (mg/square inch) is reported in the table below.

PEG1000 films containing disodium
hydrogen phosphate decahydrate particles

| | | Phosphate | | | Resulting | Solid |
|---|---|---|---|---|---|---|
| | | particles | | Spacer | film | content, |
| | PEG1000 | size, | amount, | thickness, | thickness, | mg/sq. |
| Sample | amount, g | um | g | mils | mm | inch |
| 1 | 5.0 | 100-250 | 1.25 | 20 | 0.46 | 65 |
| 2 | 5.0 | 100-250 | 2.14 | 15 | 0.33 | 70 |

### Example 13 Preparation of PEG1000 Films Comprising Particles of Bupivacaine Tridecafluorohexane-1-Sulfonate Salt

PEG1000 films comprising particles of bupivacaine tridecafluorohexane-1-sulfonate salt with salt contents of 10%, 20% and 30% wt/wt were obtained according to the procedure described in Example 2. Briefly, PEG1000 was combined with the appropriate amount of salt particles of average size 230 um at 50° C. after then films were formed on polyethylene film support using 15 mils spacer. Characteristics of the resulting films are provided in the table below.

| | | | | | Resulting | Salt |
|---|---|---|---|---|---|---|
| | | Salt particles | | Spacer | film | content, |
| | PEG1000 | size, | amount, | thickness, | thickness, | mg/sq. |
| Sample | amount, g | um | g | mils | mm | inch |
| 1 | 1.0 | 230 | 0.11 | 15 | 0.33 | 23 |
| 2 | 0.5 | | 0.13 | | | 47 |
| 3 | 0.5 | | 0.21 | | | 70 |

While this invention has been particularly shown and described with references to preferred embodiments thereof, it will be understood by those skilled in the art that various changes in form and details may be made therein without departing from the scope of the invention encompassed by the appended claims.

1. An acid addition salt of a mono- or polybasic therapeutic agent, wherein the acid is represented by Formula 1 or Formula 2: 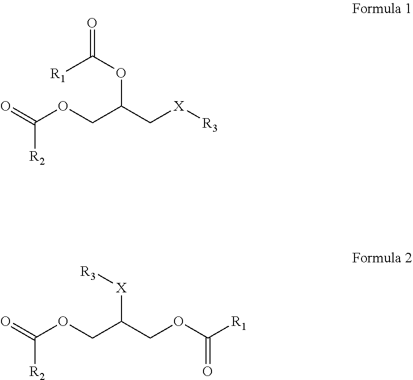
wherein
R₁ and R₂ are each independently optionally substituted C₁-C₂₄-alkyl, optionally substituted C₂-C₂₄-alkenyl, optionally substituted C₂-C₂₄-alkynyl, optionally substituted C₃-C₁₂-cycloalkyl or optionally substituted aryl;
X is O or absent and R₃ is SO₃H, P(O)(OR₄)OH or C(O)OH; or X is O and R₃ is —CH₂C(O)OH, —CH₂C(O)SH or —CH₂C(S)SH; or X is absent and R₃ is C(O)SH or C(S)SH; and
R₄ is hydrogen; optionally substituted alkyl, optionally substituted C₃-C₁₂-cycloalkyl; optionally substituted alkenyl, optionally substituted alkynyl, or optionally substituted aryl.

2. The acid addition salt of claim 1, wherein R₄ is hydrogen or C₁-C₆-alkyl.

3. The acid addition salt of claim 1, wherein the acid is represented by any one of Formulas 5 to 8:

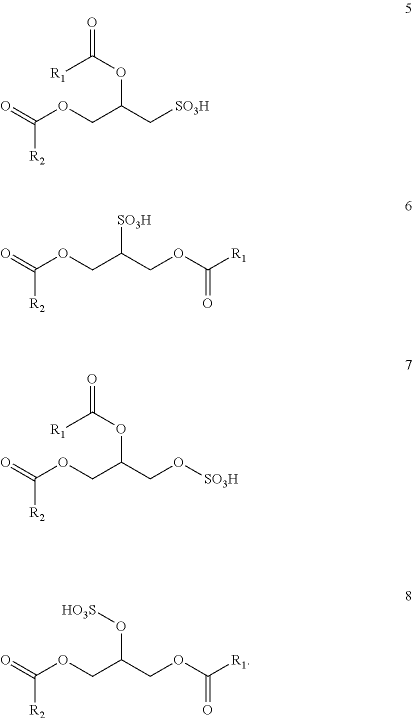

4. The acid addition salt of claim 1, wherein the acid is represented by any one of Formulas 10 to 13:

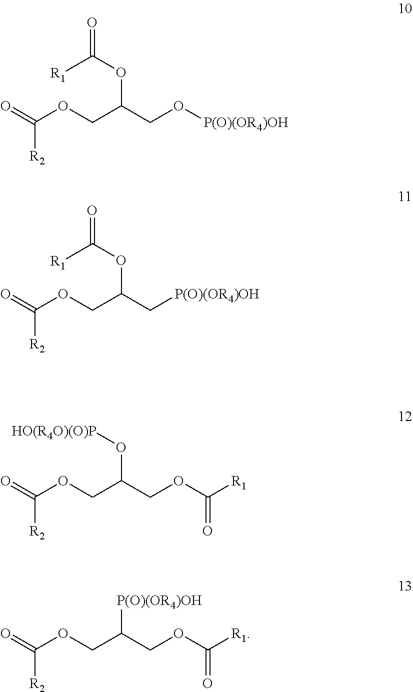

5. The acid addition salt of claim 1, wherein the acid is represented by one of Formulas 14 to 17:

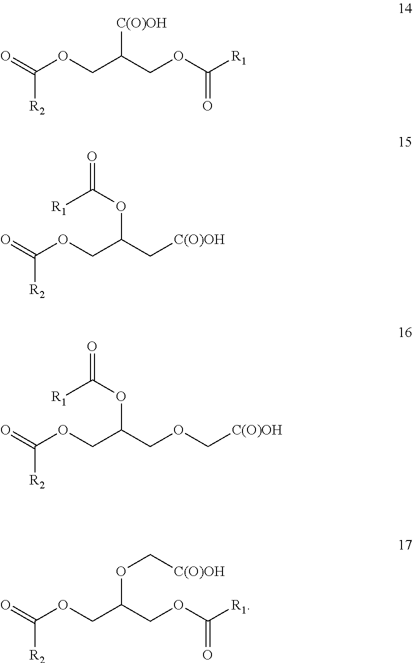

6. The acid addition salt of claim 1, wherein said basic therapeutic agent is monobasic and the salt is represented by BH⁺ A⁻, wherein BH⁺ is the cationic protonated form of the therapeutic agent and A- is the conjugate base of an acid of Formula 1 or Formula 2.

7. The acid addition salt of claim 6, wherein A- is represented by Formula 5A, 6A, 7A or 8A:

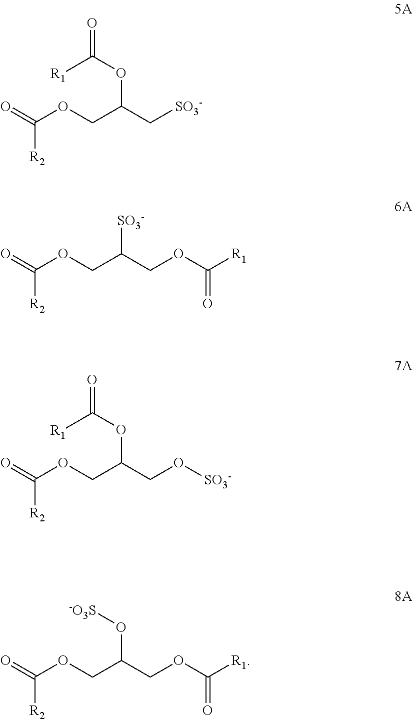

8. The acid addition salt of claim 6, wherein A- is represented by Formula 10A, 11A, 12A or 13A:

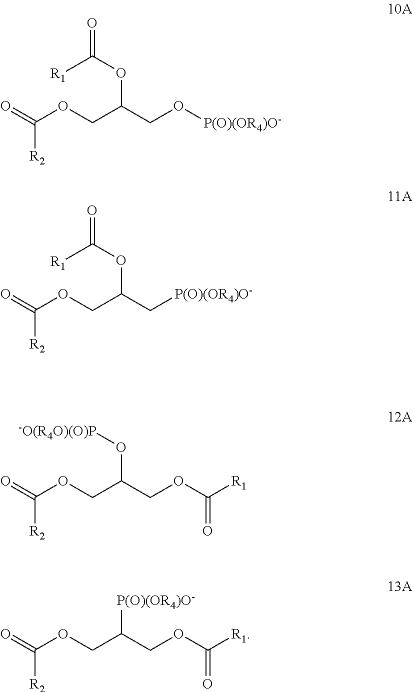

9. The acid addition salt of claim 6, wherein A- is represented by Formula 10A, 11A, 12A or 13A:

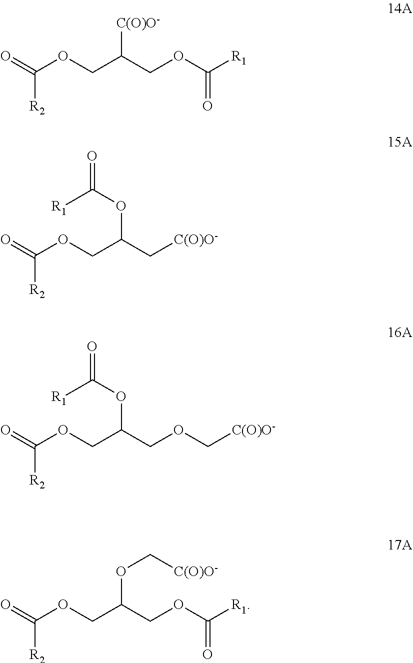

10. The acid addition salt of claim 1, wherein R₁ and R₂ are independently selected from the groups set forth in the table below:

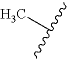

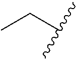 [SMILES: *CC]

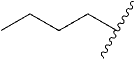 [SMILES: *CCCC]

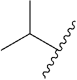 [SMILES: *C(C)C]

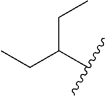 [SMILES: *C(CC)CC]

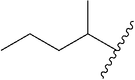 [SMILES: *C(C)CCC]

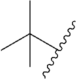 [SMILES: *C(C)(C)C]

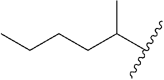 [SMILES: *C(C)CCCC]

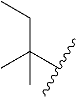 [SMILES: *C(C)(C)CC]

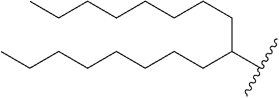 [SMILES: *C(CCCCCCCC)CCCCCCCC]

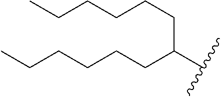 [SMILES: *C(CCCCCC)CCCCCC]

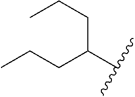 [SMILES: *C(CCC)CCC]

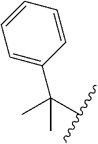 [SMILES: *C(C)(C)c1ccccc1]

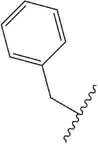 [SMILES: *Cc1ccccc1]

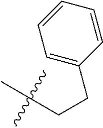 [SMILES: *CCc1ccccc1]

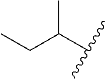 [SMILES: *C(C)CC]

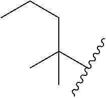 [SMILES: *C(C)(C)CCC]

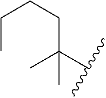 [SMILES: *C(C)(C)CCCC]

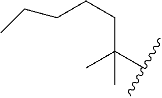 [SMILES: *C(C)(C)CCCCC]

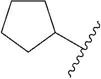 [SMILES: *C1CCCC1]

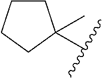 [SMILES: *C1(C)CCCC1]

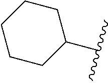 [SMILES: *C1CCCCC1]

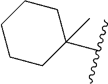 [SMILES: *C1(C)CCCCC1]

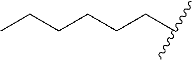 [SMILES: *CCCCCC]

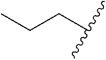 [SMILES: *CCC]

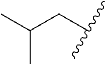 [SMILES: *CC(C)C]

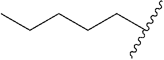 [SMILES: *CCCCC]

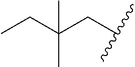 [SMILES: *CC(C)(C)CC]

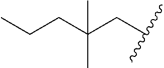 [SMILES: *CC(C)(C)CCC]

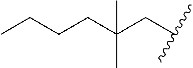 [SMILES: *CC(C)(C)CCCC]

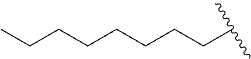 [SMILES: *CCCCCCCC]

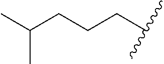 [SMILES: *CCCC(C)C]

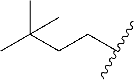 [SMILES: *CCC(C)(C)C]

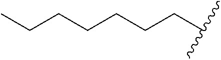 [SMILES: *CCCCCCC]

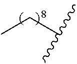

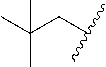 [SMILES: *CC(C)(C)C]

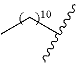

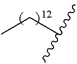

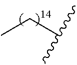

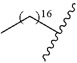

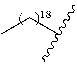

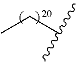

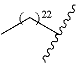

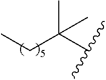

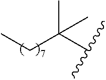

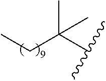

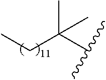

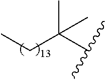

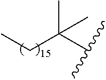

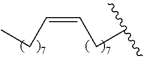

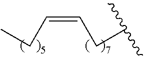

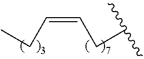

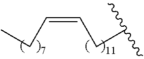

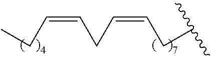

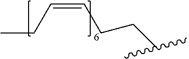

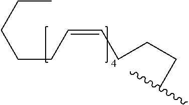

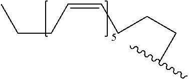

11. The acid addition salt of claim 1, wherein R₁ and R₂ are independently optionally substituted aryl or optionally substituted aryl-C₁-C₆-alkyl.

12. The acid addition salt of claim 11, wherein the optionally substituted aryl group is an optionally substituted phenyl, biphenyl, naphthyl, indenyl, indenyl, anthracenyl or phenanthryl group.

13. The acid addition salt of claim 12, wherein the optionally substituted aryl group is an optionally substituted phenyl group.

14. The acid addition salt of claim 13, wherein optionally substituted phenyl group is represented by: 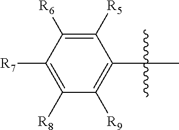
wherein R₅, R₆, R₇, R₈ and R₉ are each independently hydrogen, halogen, C₁-C₁₂-alkyl or halo-C₁-C₁₂-alkyl.

15. The acid addition salt of claim 1, wherein the therapeutic agent is a caine anesthetic.

16. The acid addition salt of claim 15, wherein the caine anesthetic is selected from the group consisting of: lidocaine (lignocaine), procaine, bupivacaine, ropivacaine, butacaine, oxybuprocaine, mepivacaine, prilocaine, amylocaine, chloroprocaine, etidocaine, propoxycaine and tropacocaine.

17. A pharmaceutical composition comprising the acid addition salt of claim 1 and a pharmaceutically acceptable carrier or excipient.

18. A pharmaceutical composition comprising the acid addition salt of claim 15 and a pharmaceutically acceptable carrier or excipient.

19. A pharmaceutical composition comprising particles comprising the acid addition salt of claim 15.

20. A method of treating pain associated with a wound, comprising administering to the wound an effective amount of the pharmaceutical composition of claim 19.

21-147. (canceled)
